(19) United States
(12) Patent Application Publication
(10) Pub. No.: US 2009/0004173 A1
(43) Pub. Date: Jan. 1, 2009

(54) Diagnosis and Treatment of Drug Resistant Leukemia

(75) Inventors: William E. Evans, Cordova, TN (US); Robert Pieters, Rotterdam (NL); Meyling H. Cheok, Memphis, TN (US); Monique L. den Boer, Rotterdam (NL); Wenjian Yang, Germantown, TN (US)

Correspondence Address: ALSTON AND BIRD LLP;ST. JUDE CHILDREN'S RESEARCH HOSPITAL, BANK OF AMERICA PLAZA, 101 SOUTH TRYON STREET, SUITE 4000, CHARLOTTE, NC, 28280-4000, US

(73) Assignee: St. Jude Children's Research Hospital, Memphis, TN (US)

(21) Appl. No.: 11/597,468

(22) PCT Filed: May 18, 2005
(86) PCT No.: PCT/US2005/017424

§ 371 Date: Aug. 12, 2008

Related U.S. Application Data
(60) Provisional application No. 60/575,762, filed on May 28, 2004.

(51) Int. Cl. A61K 38/50; G01N 33/50; C40B 30/06; A61K 31/437; C40B 40/00; A61P 35/02; A61K 31/351; A61K 31/573; C40B 30/00

(52) U.S. Cl. 424/94.6; 702/19; 506/10; 506/7; 514/285; 514/459; 506/13

(57) ABSTRACT

The present invention encompasses methods and compositions useful in the diagnosis and treatment of drug resistant leukemia. The invention provides a number of genes that are differentially expressed between drug resistant and drug sensitive acute lymphoblastic leukemia (ALL). These genes act as biomarkers for drug resistant leukemia, and further serve as molecular targets for drugs useful in treating drug resistant leukemia. Accordingly, the invention provides methods of diagnosing drug resistant leukemia and methods of selecting a therapy for subjects affected by drug-resistant leukemia. The invention also provides methods for screening for compounds for treating drug-resistant leukemia, and improved methods for treating drug-resistant leukemia. Compositions of the invention include arrays, computer readable media, and kits for use in the methods of the invention.

### FEDERALLY SPONSORED RESEARCH OR DEVELOPMENT

This invention was made in part with U.S. Government support under National Institutes of Health grant nos. R37 CA36401, R01 CA78224, RO1 CA51001, RO1 CA71907, U01 GM61393, U01 GM61394, and Cancer Center Support Grant CA21765. The U.S. Government may have certain rights in this invention.

### FIELD OF THE INVENTION

The present invention relates generally to genes associated with resistance to drugs used to treat leukemia and methods for using these genes to improve treatment of leukemia.

### BACKGROUND OF THE INVENTION

The treatment of pediatric acute lymphoblastic leukemia has improved remarkably over the past four decades, resulting in long-term disease-free survival of approximately 80 percent (Pui and Evans (1998) N. Engl. J. Med. 339:605-615 and Pui et al. (2001) Lancet Oncol. 2:597-607. Despite this progress, the number of patients with acute lymphoblastic leukemia who are not cured with contemporary therapy exceeds the total number of children with newly diagnosed acute myeloid leukemia and most other childhood cancers. Patients whose leukemia cells exhibit in vitro resistance to antileukemic agents have a significantly worse prognosis than patients whose acute lymphoblastic leukemia cells are drug sensitive (den Boer et al. (2003) J. Clin. Oncol. 21:3262-68; Kaspers et al. (1997) Blood 90:2723-29; et al. Pieters R. (1991) Lancet 338:399-403).

Little is known about the genomic determinants of leukemia cell resistance to chemotherapy. Such knowledge would provide important new insights for overcoming drug resistance in acute lymphoblastic leukemia. Accordingly, there remains a need for the identification of genes whose expression is associated with drug resistance in leukemia.

### SUMMARY OF THE INVENTION

The present invention encompasses methods and compositions useful in the diagnosis and treatment of drug resistant leukemia. The invention provides a number of genes that are differentially expressed between drug resistant and drug sensitive acute lymphoblastic leukemia (ALL). These genes act as biomarkers for drug resistant leukemia, and further serve as molecular targets for drugs useful in treating drug resistant leukemia.

Accordingly, in one embodiment the invention provides a method of diagnosing drug resistant leukemia in a subject affected by leukemia. The method comprises the steps of providing a subject expression profile of a sample from a subject affected by leukemia, providing a reference expression profile associated with resistance to at least one antileukemic agent selected from prednisolone, vincristine, L-asparaginase, and daunorubicin, and determining whether the subject expression profile shares sufficient similarity to the reference expression profile, where the subject is diagnosed with drug resistant leukemia if the subject expression profile shares sufficient statistical similarity to the reference expression profile.

The subject expression profile and the reference expression profile comprise values representing the expression levels of genes that are differentially expressed in drug-resistant versus drug-sensitive leukemia. In particular embodiments, the profiles comprise values representing the expression levels of genes selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B.

Tables 6A, 6B, 6C, 6D, 10A, and 10B provide genes that are differentially expressed in prednisolone-resistant ALL. Tables 7A, 7B, 7C, 7D, 11A, and 11B provide genes that are differentially expressed in vincristine-resistant ALL. Tables 8A, 8B, 8C, 8D, 12A, and 12B provide genes that are differentially expressed in L-asparaginase-resistant ALL. Tables 9A, 9B, 9C, 9D, 13A, and 13B provide genes that are differentially expressed in daunorubicin-resistant ALL. The invention also provides a method of determining the prognosis for a patient with leukemia or predicting whether a subject affected by leukemia has an increased risk of relapse. The method comprises the steps of providing a subject expression profile of a sample from the subject affected by leukemia, providing a reference expression profile associated with resistance to an antileukemic agent, and determining whether the subject expression profile shares sufficient similarity to the reference expression profile associated with resistance to the antileukemic agent. The subject affected by leukemia is predicted to have an increased risk of relapse if the subject expression profile shares sufficient similarity to the reference expression profile associated with resistance to the antileukemic agent.

In another embodiment, the invention provides a method of selecting a therapy for a subject affected by leukemia. The method comprises the steps of providing a subject expression profile of a sample from the subject affected by leukemia, providing a reference expression profile associated with resistance to at least one antileukemic agent selected from prednisolone, vincristine, L-asparaginase, and daunorubicin, and determining whether the subject expression profile shares sufficient similarity to the reference expression profile associated with resistance to the antileukemic agent; where the therapy selected for the subject does not comprise the antileukemic agent if the subject expression profile shares sufficient similarity to the reference expression profile associated with resistance to the antileukemic agent.

In a further aspect, the invention provides a method for screening a library of compounds to identify a compound to improve treatment of drug resistant leukemia. The method comprises the steps of providing a reference expression profile comprising one or more values representing the expression level of a gene selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B, providing a cell that is resistant to an antileukemic agent; contacting the cell with one or more compounds from the library of compounds; creating a test expression profile by determining a value representing the expression level in the cell of one or more of the genes whose expression level is represented in the reference expression profile, and determining whether the test expression profile is distinguishable the reference expression profile. If the test expression profile is distinguishable from the reference expression profile, the compound is identified as a compound useful for improving treatment of drug resistant leukemia.

In another embodiment, the invention provides a method for improving treatment of drug resistant leukemia. In one embodiment, the method comprises administering to a subject affected by drug resistant leukemia a therapy comprising an antileukemic agent and an agent that enhances the expression or activity of at least one gene selected from the genes shown in Tables 6A, 6C, 7A, 7C, 8A, 8C, 9A, 9C, 10A, 11A, 12A, and 13A. Tables 6A, 6C, and 10A provide genes whose expression is down-regulated in prednisolone-resistant ALL. Tables 7A, 7C, and 11A provide genes whose expression is down-regulated in vincristine-resistant ALL. Tables 8A, 8C, and 12A provide genes whose expression is down-regulated in L-asparaginase-resistant ALL. Tables 9A, 9C, and 13A provide genes whose expression is down-regulated in daunorubicin-resistant ALL.

In another embodiment, the method for improving treatment of drug resistant leukemia comprises administering to a subject affected by drug resistant leukemia a therapy comprising an antileukemic agent and an agent that inhibits the expression or activity of one or more genes selected from the genes shown in Tables 6B, 6D, 7B, 7D, 8B, 8D, 9B, 9D, 10B, 11B, 12B, and 13B. Tables 6B, 6D, and 10B provide genes whose expression is up-regulated in prednisolone-resistant ALL. Tables 7B, 7D, and 11B provide genes whose expression is up-regulated in vincristine-resistant ALL. Tables 8B, 8D, and 12B provide genes whose expression is up-regulated in L-asparaginase-resistant ALL. Tables 9B, 9D, and 13B provide genes whose expression is up-regulated in daunorubicin-resistant ALL.

The invention also provides an array for use in a method of diagnosing drug resistant leukemia. The array comprises a substrate having a plurality of addresses, where each address has a capture probe that can specifically bind to a nucleic acid molecule selected from the group consisting of genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B.

The invention also provides a computer-readable medium comprising digitally-encoded expression profiles having values representing the expression of a gene selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B.

In another embodiment, the invention provides a kit for diagnosing drug-resistant leukemia. The kit comprises (1) an array having a substrate with of addresses, where each address has a capture probe that can specifically bind a nucleic acid molecule selected from the group consisting of genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B; and (2) a computer-readable medium comprising digitally-encoded expression profiles having values representing the expression of a gene selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B.

### DESCRIPTION OF THE FIGURE

FIG. 1 shows a Kaplan-Meier analysis of treatment outcome among patients with gene expression patterns associated with cellular resistance or sensitivity to the four antileukemic agents. Panel (a) shows disease-free survival of patients treated on the Dutch and COALL protocols. Patients are sub-grouped based on combined drug resistance gene expression scores of 172 gene probe sets for antileukemic agents (prednisolone, vincristine, L-asparaginase and daunorubicin). The 33 percent with the lowest score (Sensitive), 33 percent with an intermediate (Intermediate) and 33 percent with the highest score (Resistant) are shown. Panel B shows disease-free survival of patients treated on St. Jude Children's Research Hospital protocols. Patients were assigned to the Sensitive, Intermediate and Resistant categories using the combined drug resistance gene expression score (172 gene probe sets for four drugs) according to the same values used to assign the Dutch and COALL patients to one of these categories (panel a).

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the invention. Although specific terms are employed herein, they are used in a generic and descriptive sense only and not for purposes of limitation.

In the present invention, genes that are differentially expressed between drug resistant and drug sensitive leukemia are identified. These genes may be used as biomarkers for diagnosing drug resistant leukemia, and for selecting a therapy for a patient having drug resistant leukemia. The differentially expressed genes are also useful in a screening method to identify compounds that increase sensitivity to antileukemic drugs. In addition, the identified genes may serve as molecular targets for drugs useful in treating drug resistant leukemia. Accordingly, the present invention encompasses methods and compositions useful in the diagnosis and treatment of drug resistant leukemia.

### Diagnostic Methods:

In one embodiment, the present invention provides a method of diagnosing drug resistant leukemia in a subject affected by leukemia. The subject affected by leukemia may be either a pediatric leukemia patient or an adult pediatric patient. By “leukemia,” it is intended a malignant proliferation of the leukopoietic tissues. In some embodiments, the leukemia is acute lymphoblastic leukemia (ALL) or acute myeloblastic leukemia (AML). In particular embodiments, the leukemia is ALL.

By “drug resistant leukemia,” it is intended leukemia in which the leukemia cells are resistant to being killed by the concentrations of antileukemic agents that are used to kill leukemia cells in drug-sensitive leukemia. In particular embodiments, the drug or drugs for which resistance is to be determined is selected from prednisolone, vincristine, L-asparaginase, and daunorubicin. The relative resistance of a leukemia cell to a drug may be determined by calculating the drug concentration that is lethal to 50% of the leukemia cells (LC-50). For the purposes of the present invention, a leukemia cell is “resistant” to a drug if the LC-50 value is equal to or greater than the value shown in the chart below:

LC-50 values for classifying leukemia
cells as drug-resistant

| Drug | LC-50 | |
|---|---|---|
| Prednisolone | ≧150 | μg/ml |
| Vincristine | ≧1.758 | μg/ml |
| L-asparaginase | ≧0.912 | IU/ml |
| Daunorubicin | ≧0.114 | μg/ml |

The diagnostic method comprises the steps of providing a subject expression profile of a sample from a subject affected by leukemia, providing a reference expression profile associated with resistance to at least one antileukemic agent selected from prednisolone, vincristine, L-asparaginase, and daunorubicin, and determining whether the subject expression profile shares sufficient similarity to the reference expression profile, where the subject affected by leukemia is diagnosed with drug resistant leukemia if the subject expression profile shares sufficient similarity to the reference expression profile.

The subject expression profile and the reference expression profile comprise values representing the expression levels of genes that are differentially expressed in drug-resistant versus drug-sensitive leukemia. In particular embodiments, the profiles comprise values representing the expression levels of genes selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B.

Tables 6A, 6B, 6C, 6D, 10A, and 10B provide genes whose expression is differentially regulated in prednisolone-resistant ALL. Accordingly, in some embodiments, the antileukemic agent is prednisolone and the subject expression profile and reference expression profile contain genes selected from the genes shown in Table 6A, 6B, 6C, 6D, 10A, and 10B.

Tables 7A, 7B, 7C, 7D, 11A, and 11B provide genes whose expression is differentially regulated in vincristine-resistant ALL. Accordingly, in some embodiments, the antileukemic agent is vincristine and the subject expression profile and reference expression profile contain genes selected from the genes shown in Table 7A, 7B, 7C, 7D, 11A, and 11B.

Tables 8A, 8B, 8C, 8D, 12A, and 12B provide genes whose expression is differentially regulated in L-asparaginase-resistant ALL. Thus, in some embodiments, the antileukemic agent is L-asparaginase and the subject expression profile and reference expression profile contain genes selected from the genes shown in Table 8A, 8B, 8C, 8D, 12A, and 12B.

Tables 9A, 9B, 9C, 9D, 13A, and 13B provide genes whose expression is differentially regulated in daunorubicin-resistant ALL. In some embodiments, the antileukemic agent is daunorubicin and the subject expression profile and reference expression profile contain genes selected from the genes shown in 9A, 9B, 9C, 9D, 13A, and 13B.

In another embodiment, the invention provides a method of selecting a therapy for a subject affected by leukemia. The method comprises the steps of providing a subject expression profile of a sample from the subject affected by leukemia, providing a reference expression profile associated with resistance to at least one antileukemic agent selected from prednisolone, vincristine, L-asparaginase, and daunorubicin, and determining whether the subject expression profile shares sufficient similarity to the reference expression profile associated with resistance to the antileukemic agent, where the therapy selected for the subject does not comprise the antileukemic agent if the subject expression profile shares sufficient similarity to the reference expression profile associated with resistance to the antileukemic agent.

In a related embodiment, the method of selecting a therapy for a subject affected by leukemia comprises the steps of providing a subject expression profile of a sample from the subject affected by leukemia, providing a reference expression profile associated with resistance to at least one antileukemic agent selected from prednisolone, vincristine, L-asparaginase, and daunorubicin, and determining whether the subject expression profile is distinguishable from the reference expression profile associated with resistance to the antileukemic agent. If the subject expression profile shares statistically significant similarity with the reference profile, then the antileukemic agent is not selected for therapy for the subject.

In these methods, the subject expression profile and the reference expression profile comprise one or more values representing the expression level of a gene having differential expression in subjects affected by drug-resistant leukemia. In particular embodiments, the profiles comprise values representing the expression levels of genes selected from the genes shown in Tables 6A, 6B, 6C, 6D, 7A, 7B, 7C, 7D, 8A, 8B, 8C, 8D, 9A, 9B, 9C, 9D, OA, 10B, 11A, 11B, 12A, 12B, 13A, and 13B.

A description of methods of making and comparing expression profiles is provided elsewhere herein.

### Methods of Screening for Compounds to Improve the Treatment of Drug-Resistant Leukemia

In a further aspect, the invention provides a method for screening a library of test compounds to identify a candidate compound to improve treatment of drug resistant leukemia. In one embodiment, the method comprises the steps of providing a reference expression profile associate with drug resistance, where the reference expression profile comprises one or more values representing the expression level of a gene that is differentially expressed in drug resistant leukemia, providing a cell that is resistant to an antileukemic agent; contacting the cell with one or more compounds from the library of compounds; creating a test expression profile by determining a value representing the expression level in the cell of one or more of the genes whose expression level is represented in the reference expression profile and determining whether the test expression profile is statistically distinguishable the reference expression profile. If the test expression profile is statistically distinguishable from the reference expression profile, then the compound is identified as a compound useful for improving treatment of drug resistant leukemia.

In another embodiment, the method comprises the steps of providing a reference expression profile associated with drug sensitivity, where the reference profile comprises one or more values representing the expression level of a gene that is differentially expressed in drug resistant leukemia, providing a cell that is resistant to an antileukemic agent; contacting the cell with one or more compounds from the library of compounds; creating a test expression profile by determining a value representing the expression level in the cell of one or more of the genes whose expression level is represented in the reference expression profile and determining whether the test expression profile shares statistically significant similarity to the reference expression profile. If the test expression profile shares statistically significant similarity with the reference expression profile, then the compound is identified as a compound useful for improving treatment of drug resistant leukemia.

In some embodiments, the test expression profile and the reference expression profile comprise values representing the expression of genes selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B. The genes whose expression level is measured to generate the profile will be selected based on the resistance profile of the cell. For example, if the cell is resistant to prednisolone, genes from Tables 6A, 6B, 6C, 6D, 10A, and 10B can be used. Similarly, if the cell is resistant to vincristine, genes from Tables 7A, 7B, 7C, 7D, 11A, and 11B can be used. If the cell is resistant to L-asparaginase, genes from Tables 8A, 8B, 8C, 8D, 12A, and 12B can be used. If the cell is resistant to daunorubicin, genes from Tables 9A, 9B, 9C, 9D, 13A, and 13B can be used.

The cell that is resistant to an antileukemic agent can be derived from a variety of sources including, but not limited to, single cells, a collection of cells, tissue, cell culture, bone marrow, blood, or other bodily fluids. The tissue or cell source may include a tissue biopsy sample, a cell sorted population, cell culture, or a single cell. Sources for the sample of the present invention include cells from peripheral blood or bone marrow, such as blast cells from peripheral blood or bone marrow.

In some embodiments, an expression profile is produced for the drug-resistant cell before and after it is contacted with the antileukemic agent. In this embodiment, the expression profile produced from the cell prior to contact with the test compound is the reference profile associated with drug resistance used in the method. The test expression profile generated after the contact with the compound is then compared to this reference expression profile. If the test compound alters the expression of genes associated with drug resistance such that the post-contact test expression profile is statistically distinguishable from the pre-contact reference expression profile, then the compound is identified as a candidate compound for the treatment of drug resistant leukemia. In other embodiments, the reference expression profile is an expression profile that has a statistically significant correlation with drug resistance or with drug sensitivity, but is not produced directly from the drug resistant cell.

A description of expression profiles and test compounds that may be screened according to the invention is provided elsewhere herein.

### Methods of Improving Treatment of Drug-Resistant Leukemia

In one aspect, the invention provides a method for improving treatment of drug resistant leukemia. This method is based on the identification of specific genes that are either significantly up-regulated or significantly down-regulated in cells that are resistant to particular antileukemic agents. Changes in the expression of these genes are associated with drug resistant in leukemia cells. Accordingly, drug resistance in leukemia cells can be modulated by enhancing the expression or activity of down-regulated genes, or by inhibiting the expression or activity of up-regulated genes.

Accordingly, in one embodiment, the method comprises administering to a subject affected by drug resistant leukemia a therapy comprising an antileukemic agent and a second agent that enhances the expression or activity of at least one gene that is down-regulated in drug resistant leukemia. The gene that is down-regulated in drug resistant leukemia is selected from the genes shown in Tables 6A, 6C, 7A, 7C, 8A, 8C, 9A, 9C, 10A, 11A, 12A, and 13A.

Tables 6A, 6C, and 10A provide genes whose expression is down-regulated in prednisolone-resistant ALL. Accordingly, these genes and their expression products are targets for up-regulation in treating resistance to prednisolone.

Tables 7A, 7C, and 11A provide genes whose expression is down-regulated in vincristine-resistant ALL. Accordingly, these genes and their expression products are targets for up-regulation in treating resistance to vincristine.

Tables 8A, 8C, and 12A provide genes whose expression is down-regulated in L-asparaginase-resistant ALL. Accordingly, these genes and their expression products are targets for up-regulation in treating resistance to L-asparaginase.

Tables 9A, 9C, and 13A provide genes whose expression is down-regulated in daunorubicin-resistant ALL. Accordingly, these genes and their expression products are targets for up-regulation in treating resistance to daunorubicin.

In another embodiment of the method for improving treatment of drug resistant leukemia comprises administering to a subject affected by drug resistant leukemia a therapy comprising an antileukemic agent and an agent that inhibits the expression or activity of at least one gene selected from the genes shown in Tables 6B, 6D, 7B, 7D, 8B, 8D, 9B, 9D, 10B, 11B, 12B, and 13B.

Tables 6B, 6D, and 10B provide genes whose expression is up-regulated in prednisolone-resistant ALL. Accordingly, these genes and their expression products are targets for inhibition in treating resistance to prednisolone.

Tables 7B, 7D, and 11B provide genes whose expression is up-regulated in vincristine-resistant ALL. Accordingly, these genes and their expression products are targets for inhibition in treating resistance to vincristine.

Tables 8B, 8D, and 12B provide genes whose expression is up-regulated in L-asparaginase-resistant ALL. Accordingly, these genes and their expression products are targets for inhibition in treating resistance to L-asparaginase.

Tables 9B, 9D, and 13B provide genes whose expression is up-regulated in daunorubicin-resistant ALL. Accordingly, these genes and their expression products are targets for inhibition in treating resistance to daunorubicin.

### Expression Profiles

As used herein, an “expression profile” comprises one or more values corresponding to a measurement of the relative abundance of a gene expression product. Such values may include measurements of RNA levels or protein abundance. Thus, the expression profile can comprise values representing the measurement of the transcriptional state or the translational state of the gene. See, U.S. Pat. Nos. 6,040,138, 5,800,992, 6,020135, 6,344,316, and 6,033,860, which are hereby incorporated by reference in their entireties.

The transcriptional state of a sample includes the identities and relative abundance of the RNA species, especially mRNAs present in the sample. Preferably, a substantial fraction of all constituent RNA species in the sample are measured, but at least a sufficient fraction to characterize the transcriptional state of the sample is measured. The transcriptional state can be conveniently determined by measuring transcript abundance by any of several existing gene expression technologies.

Translational state includes the identities and relative abundance of the constituent protein species in the sample. As is known to those of skill in the art, the transcriptional state and translational state are related.

In some embodiments, the expression profiles of the present invention are generated from samples from subjects affected by leukemia or drug-resistant leukemia, including subjects having leukemia or drug-resistant leukemia, subjects suspected of having leukemia, subjects having a propensity to develop leukemia or drug-resistant leukemia, or subjects who have previously had leukemia or drug-resistant leukemia, or subjects undergoing therapy for leukemia or drug-resistant leukemia. The samples from the subject used to generate the expression profiles of the present invention can be derived from a variety of sources including, but not limited to, single cells, a collection of cells, tissue, cell culture, bone marrow, blood, or other bodily fluids. The tissue or cell source may include a tissue biopsy sample, a cell sorted population, cell culture, or a single cell. Sources for the sample of the present invention include cells from peripheral blood or bone marrow, such as blast cells from peripheral blood or bone marrow.

In selecting a sample, the percentage of the sample that constitutes cells having differential gene expression in drug resistant versus drug sensitive leukemia should be considered. Samples may comprise at least 20%, at least 30%, at least 40%, at least 50%, at least 55%, at least 60%, at least 70%, at least 75%, at least 80%, at least 85%, at least 90%, or at least 95% cells having differential expression in drug resistant versus drug sensitive leukemia, with a preference for samples having a higher percentage of such cells. In some embodiments, these cells are blast cells, such as leukemic cells. The percentage of a sample that constitutes blast cells may be determined by methods well known in the art.

In some embodiments of the present invention, the expression profiles comprise values representing the expression levels of genes that are differentially expressed in drug resistant leukemia. The term “differentially expressed” as used herein means that the measurement of a cellular constituent varies in two or more samples. The cellular constituent may be up-regulated in a sample from a subject having one physiologic condition in comparison with a sample from a subject having a different physiologic condition, or down-regulated in a sample from a subject having one physiologic condition in comparison with a sample from a subject having a different physiologic condition. The differentially expressed genes of the present invention are expressed at different levels in drug resistant leukemia and drug sensitive leukemia. Some of the differentially expressed genes are up-regulated in lymphoblasts from subjects having drug-resistant leukemia in comparison with the expression level of the same gene in drug-sensitive leukemia, while other genes are down-regulated in lymphoblasts from subjects having drug resistant leukemia in comparison with the same gene in subjects having drug sensitive leukemia. These differentially expressed genes were identified based on gene expression levels for 14,550 probes in 173 leukemia samples.

The invention provides genes that are differentially expressed in lymphoblasts that are resistant to one or more of four different antileukemic agents, prednisolone, vincristine, L-asparaginase, and daunorubicin. Tables 6A, 6B, 6C, 6D, 10A, and 10B provide genes whose expression is differentially regulated in prednisolone-resistant ALL. Tables 6A, 6C, and 10A provide genes whose expression is down-regulated in prednisolone-resistant ALL in comparison with prednisolone-sensitive ALL, while Tables 6B, 6D, and 10B provide genes whose expression is up-regulated in prednisolone-resistant ALL in comparison with prednisolone-sensitive ALL. Tables 7A, 7B, 7C, 7D, 11A, and 11B provide genes whose expression is differentially regulated in vincristine resistant ALL. Tables 7A, 7C, and 11A provide genes whose expression is down-regulated in vincristine-resistant ALL in comparison with vincristine-sensitive ALL, while Tables 7B, 7D, and 11B provide genes whose expression is up-regulated in vincristine-resistant ALL in comparison with vincristine-sensitive ALL. Tables 8A, 8B, 8C, 8D, 12A, and 12B provide genes whose expression is differentially regulated in L-asparaginase resistant ALL. Tables 8A, 8C, and 12A provide genes whose expression is down-regulated in L-asparaginase-resistant ALL in comparison with L-asparaginase-sensitive ALL, while Tables 8B, 8D, and 12B provide genes whose expression is up-regulated in L-asparaginase-resistant ALL in comparison with L-asparaginase-sensitive ALL. Tables 9A, 9B, 9C, 9D, 13A, and 13B provide genes whose expression is differentially regulated in daunorubicin resistant ALL. Tables 9A, 9C, and 13A provide genes whose expression is down-regulated in daunorubicin-resistant ALL in comparison with daunorubicin-sensitive ALL, while Tables 9B, 9D, and 13B provide genes whose expression is up-regulated in daunorubicin-resistant ALL in comparison with daunorubicin-sensitive ALL.

The expression profiles according to the invention comprise one or more values representing the expression level of a gene having differential expression in drug resistant ALL. Each expression profile contains a sufficient number of values such that the profile can be used to distinguish drug resistant leukemia from drug sensitive leukemia. In some embodiments, the expression profiles comprise only one value. In other embodiments, the expression profile comprises more than one value corresponding to a differentially expressed gene, for example at least 2 values, at least 3 values, at least 4 values, at least 5 values, at least 6 values, at least 7 values, at least 8 values, at least 9 values, at least 10 values, at least 11 values, at least 12 values, at least 13 values, at least 14 values, at least 15 values, at least 16 values, at least 17 values, at least 18 values, at least 19 values, at least 20 values, at least 22 values, at least 25 values, at least 27 values, at least 30 values, at least 35 values, at least 40 values, at least 45 values, at least 50 values, at least 75 values, at least 100 values, at least 125 values, at least 150 values, at least 175 values, at least 200 values, at least 250 values, at least 300 values, at least 400 values, at least 500 values, at least 600 values, at least 700 values, at least 800 values, at least 900 values, at least 1000 values, at least 1200 values, at least 1500 values, or at least 2000 or more values.

It is recognized that the diagnostic accuracy of diagnosing drug resistant leukemia or predicting a prognosis for a leukemia patient will vary based on the number of values contained in the expression profile. Generally, the number of values contained in the expression profile is selected such that the diagnostic accuracy is at least at least 65%, at least 70%, at least 75%, at least 80%, at least 85%, at least 87%, at least 90%, at least 91%, at least 92%, at least 93%, at least 94%, at least 95%, at least 96%, at least 97%, at least 98%, or at least 99%, as calculated using methods described elsewhere herein, with an obvious preference for higher percentages of diagnostic accuracy.

It is recognized that the accuracy of diagnosing drug-resistant leukemia or determining the prognosis for a patient will vary based on the strength of the correlation between the expression levels of the differentially expressed genes and the associated physiologic condition. When the values in the expression profiles represent the expression levels of genes whose expression is strongly correlated with the physiologic condition, it may be possible to use fewer number of values in the expression profile and still obtain an acceptable level of diagnostic or prognostic accuracy.

The strength of the correlation between the expression level of a differentially expressed gene and the presence or absence of a particular physiologic state may be determined by a statistical test of significance. Methods for determining the strength of a correlation between the expression level of a differentially-expressed gene and a particular physiologic state by assigning a statistical score to the correlation are reviewed in Holloway et al. (2002) Nature Genetics Suppl. 32:481-89, Churchill (2002) Nature Genetics Suppl. 32:490-95, Quackenbush (2002) Nature Genetics Suppl. 32: 496-501; Slonim (2002) Nature Genetics Suppl. 32:502-08; and Chuaqui et al. (2002) Nature Genetics Suppl. 32:509-514; each of which is herein incorporated by reference in its entirety. The statistical scores may be used to select the genes whose expression levels have the greatest correlation with a particular physiologic state in order to increase the diagnostic or prognostic accuracy of the methods of the invention, or in order to reduce the number of values contained in the expression profile while maintaining the diagnostic or prognostic accuracy of the expression profile. By a gene whose expression level is “correlated with” a particular physiologic state, it is intended a gene whose expression shows a statistically significant correlation with the physiologic state. Such methods may be used to select the genes whose expression levels have the greatest correlation with a particular treatment outcome in order to increase the predictive accuracy of the methods of the invention.

The values in the expression profiles of the invention are measurements representing the absolute or the relative expression level of differentially expressed genes. The expression levels of these genes may be determined by any method known in the art for assessing the expression level of an RNA or protein molecule in a sample. For example, expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are expressly incorporated herein by reference. The gene expression monitoring system may also comprise nucleic acid probes in solution. Expression levels of RNA may also be monitored using the reverse transcriptase polymerase chain reaction (e.g., TaqMan®).

In one embodiment of the invention, microarrays are used to measure the values to be included in the expression profiles. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, the Experimental section. See also, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

In one approach, total mRNA isolated from the sample is converted to labeled cRNA and then hybridized to an oligonucleotide array. Each sample is hybridized to a separate array. Relative transcript levels are calculated by reference to appropriate controls present on the array and in the sample.

In another embodiment, the values in the expression profile are obtained by measuring the abundance of the protein products of the differentially-expressed genes. The abundance of these protein products can be determined, for example, using antibodies specific for the protein products of the differentially-expressed genes. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments, which can be generated by treating the antibody with an enzyme such as pepsin.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length protein product from a differentially-expressed gene, or an antigenic peptide fragment of the protein product can be used as an immunogen. Preferred epitopes encompassed by the antigenic peptide are regions of the protein product of the differentially expressed gene that are located on the surface of the protein, e.g., hydrophilic regions, as well as regions with high antigenicity. The antibody can be used to detect the protein product of the differentially expressed gene in order to evaluate the abundance and pattern of expression of the protein. These antibodies can also be used diagnostically to monitor protein levels in tissue as part of a clinical testing procedure, e.g., to, for example, determine the efficacy of a given therapy. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Once the values comprised in the subject expression profile and the reference expression profile or expression profiles are established, the subject profile is compared to the reference profile to determine whether the subject expression profile is sufficiently similar to the reference profile. Alternatively, the subject expression profile is compared to a plurality of reference expression profiles to select the reference expression profile that is most similar to the subject expression profile.

Any method known in the art for comparing two or more data sets to detect similarity between them may be used to compare the subject expression profile to the reference expression profiles. To determine whether two or more expression profiles show statistically significant similarity, statistical tests may be performed to determine whether any differences between the expression profile are likely to have been achieved by a random event. Methods for comparing gene expression profiles to determine whether they share statistically significant similarity are known in the art and also reviewed in Holloway et al. (2002) Nature Genetics Suppl. 32:481-89, Churchill (2002) Nature Genetics Suppl. 32:490-95, Quackenbush (2002) Nature Genetics Suppl. 32: 496-501; Slonim (2002) Nature Genetics Suppl. 32:502-08; and Chuaqui et al. (2002) Nature Genetics Suppl. 32:509-514; each of which is herein incorporated by reference in its entirety. An expression profile is “distinguishable” or “statistically distinguishable” from a reference profile according to the invention if the two expression profiles do not share statistically significant similarity.

The accuracy of diagnosing a subject with drug resistant leukemia or predicting a prognosis for a leukemia patient by comparing an expression profile for the subject with reference expression profile associated with drug resistant depends in part on the degree of similarity between the two profiles. Therefore, are required, the stringency with which the similarity between the subject expression profile and the reference profile is evaluated should be increased. For example, in various embodiments, the p-value obtained when comparing the subject expression profile to a reference profile that shares sufficient similarity with the subject expression profile is less than 0.20, less than 0.15, less than 0.10, less than 0.09, less than 0.08, less than 0.07, less than 0.06, less than 0.05, less than 0.04, less than 0.03, less than 0.02, or less than 0.01.

In some embodiments, the expression profiles of the invention are used to select a therapy for a leukemia patient. A therapy, as used herein, refers to a course of treatment intended to reduce or eliminate the affects or symptoms of a disease, in this case leukemia. A therapy regimen will typically comprise, but is not limited to, a prescribed dosage of one or more drugs or hematopoietic stem cell transplantation. Therapies, ideally, will be beneficial and reduce the disease state but in many instances the effect of a therapy will have non-desirable effects as well. Thus, the methods of the invention are useful for monitoring the effectiveness of a therapy even when non-desirable side-effects are observed.

### Arrays, Computer-Readable Medium, and Kits

The present invention provides compositions that are useful in diagnosing drug resistant leukemia and in screening for drugs to treat drug-resistant leukemia. These compositions include arrays comprising a substrate having a capture probes that can bind specifically to nucleic acid molecules that are differentially expressed in drug resistant leukemia. In another aspect, the invention also provides a computer-readable medium having digitally encoded reference profiles useful in the methods of the claimed invention. The invention also encompasses kits comprising an array of the invention and a computer-readable medium having digitally-encoded reference profiles with values representing the expression of nucleic acid molecules detected by the arrays.

The arrays of the invention comprise capture probes for detecting the differentially expressed genes of the invention. By “array” is intended a solid support or substrate with peptide or nucleic acid probes attached to the support or substrate. Arrays typically comprise a plurality of different nucleic acid or peptide capture probes that are coupled to a surface of a substrate in different, known locations. These arrays, also described as “microarrays” or colloquially “chips” have been generally described in the art, for example, in U.S. Pat. Nos. 5,143,854, 5,445,934, 5,744,305, 5,677,195, 6,040,193, 5,424,186, 6,329,143, and 6,309,831 and Fodor et al. (1991) Science 251:767-77, each of which is incorporated by reference in its entirety. These arrays may generally be produced using mechanical synthesis methods or light directed synthesis methods, which incorporate a combination of photolithographic methods and solid phase synthesis methods.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

The arrays provided by the present invention comprise capture probes that can specifically bind a nucleic acid molecule that is differentially expressed in leukemia risk groups, a nucleic acid molecule that is differentially expressed in drug resistant leukemia. The capture probes are designed to hybridize to target nucleic acid molecules corresponding to messenger RNAs of differentially expressed genes (such as cDNA copies of differentially expressed messenger RNAs) and allow their detection. Method of designing a probe that will hybridize with a target nucleic acid molecule are well know in the art. Any capture probe that detects a differentially expressed gene of the invention may be used in an array.

The arrays may also comprise capture probes that bind to control nucleic acid molecules. The control nucleic acid molecules can be used to normalize expression data obtained from the arrays, allowing experiments performed at different times using different arrays to be compared.

The arrays can be used to measure the expression levels of nucleic acid molecules to thereby create an expression profile for use in methods of determining the diagnosis and prognosis for leukemia patients, and in screening for compounds to improve treatment of drug-resistant leukemia.

In some embodiments, each capture probe in the array detects a nucleic acid molecule selected from the nucleic acid molecules designated in 6A, 6B, 6C, 6D, 7A, 7B, 7C, 7D, 8A, 8B, 8C, 8D, 9A, 9B, 9C, 9D, 10A, 10B, 11A, 11B 12A, 12B, 13A, and 13B. The designated nucleic acid molecules include those differentially expressed in prednisolone-resistant ALL (Tables 6A, 6B, 6C, 6D, 10A, and 10B); vincristine-resistant ALL (Tables 7A, 7B, 7C, 7D, 11A, and 11B), L-asparaginase-resistant ALL (Tables 8A, 8B, 8C, 8D, 12A, and 12B), and daunorubicin-resistant ALL (Tables 9A, 9B, 9C, 9D, 13A, and 13B).

The arrays of the invention comprise a substrate having a plurality of addresses, where each addresses has a capture probe that can specifically bind a target nucleic acid molecule. The number of addresses on the substrate varies with the purpose for which the array is intended. The arrays may be low-density arrays or high-density arrays and may contain 4 or more, 8 or more, 12 or more, 16 or more, 20 or more, 24 or more, 32 or more, 48 or more, 64 or more, 72 or more 80 or more, 96, or more addresses, or 192 or more, 288 or more, 384 or more, 768 or more, 1536 or more, 3072 or more, 6144 or more, 9216 or more, 12288 or more, 15360 or more, or 18432 or more addresses. In some embodiments, the substrate has no more than 12, 24, 48, 96, or 192, or 384 addresses, no more than 500, 600, 700, 800, or 900 addresses, or no more than 1000, 1200, 1600, 2400, or 3600 addressees.

The invention also provides a computer-readable medium comprising one or more digitally-encoded expression profiles, where each profile has one or more values representing the expression of a gene that is differentially expressed in a drug resistant leukemia. Thus, in one embodiment, the invention encompasses a computer-readable medium comprising digitally-encoded expression profiles having values representing the expression of a gene selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B. In some embodiments, the digitally-encoded expression profiles are comprised in a database. See, for example, U.S. Pat. No. 6,308,170.

The present invention also provides kits useful for diagnosing drug resistant leukemia, and for screening for drugs for treating drug resistant leukemia. These kits comprise an array and a computer readable medium. The array comprises a substrate having addresses, where the addresses have capture probes that can specifically bind nucleic acid molecules that are differentially expressed in drug resistant leukemia. The computer-readable medium has digitally-encoded expression profiles containing values representing the expression level of a nucleic acid molecule detected by the array. In some embodiments, the expression profile is a reference expression profile associated with drug-resistant leukemia. The array can be used to produce a test expression profile from a sample, and this test expression profile can then be compared to the reference profile or profiles contained in the computer readable medium to determine whether it the test profile shares similarity with the reference profile.

Thus, in one embodiment, the kit comprises (1) an array having a substrate with of addresses, where each address has a capture probe that can specifically bind a nucleic acid molecule selected from the group consisting of genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B; and (2) a computer-readable medium comprising digitally-encoded expression profiles having values representing the expression of a gene selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B.

The kits of the invention may also include methods for use in a method of diagnosing drug resistant leukemia, a method of predicting the prognosis for a leukemia patient, or a method for screening for compounds for use in improving treatment of drug leukemia. These methods are described elsewhere herein.

### Methods of Screening and Therapeutic Targets

The methods and compositions of the invention may be used to screen test compounds to identify therapeutic compounds useful for the treatment of drug-resistant leukemia. In one embodiment, the test compounds are screened in a sample comprising drug-resistant primary cells representing drug resistant leukemia. After exposure to the test compound, the expression levels in the sample of one or more of the differentially-expressed genes of the invention are measured using methods described elsewhere herein. Values representing the expression levels of the differentially-expressed genes are used to generate a test expression profile. This test expression profile is then compared to a reference expression profile associated with drug-resistant leukemia to determine the similarity between the subject expression profile and the reference expression profile. If the test expression profile is distinguishable from the drug resistant reference expression profile, and shares similarity with an expression profile from a drug-sensitive sample, the test compound is identified as a candidate compound useful for the treatment of drug-resistant leukemia.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam (1997) Anticancer Drug Des. 12:145).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (U.S. Pat. No. 5,223,409), spores (U.S. Pat. No. 5,223,409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. U.S.A. 97:6378-6382); (Felici (1991) J. Mol. Biol. 222:301-310).

Candidate compounds include, for example, 1) peptides such as soluble peptides, including Ig-tailed fusion peptides and members of random peptide libraries (see, e.g., Lam et al. (1991) Nature 354:82-84; Houghten et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries; 5) zinc analogs; 6) leukotriene A₄ and derivatives; 7) classical aminopeptidase inhibitors and derivatives of such inhibitors, such as bestatin and arphamenine A and B and derivatives; 8) and artificial peptide substrates and other substrates, such as those disclosed herein above and derivatives thereof.

The present invention discloses a number of genes that are differentially expressed in drug resistant leukemia. These differentially-expressed genes are shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B. Because the expression of these genes is associated with drug resistant leukemia, these genes may play a role in resistance to antileukemic agents. Accordingly, these genes and their gene products are potential therapeutic targets that are useful in methods of screening test compounds to identify therapeutic compounds for the treatment of leukemia.

The differentially expressed genes and their expression products identified as targets in accordance with the invention may be used in conventional biochemical assays or in cell-based screening assays. Johnston, P. A. and Johnston, P. A., “Cellular Platforms for HTS: three case studies”, Drug Discovery Today 7 (6): 353-363 (March 2002); Drews, J., “Drug discovery: a historical perspective”, Science 287: 1960-1965 (2000); Valler, M. J. and Green, D., “Diversity screening versus focused screening in drug discovery”, Drug Discovery Today 5 (7): 286-293 (2000); Grepin, C. and Pernelle, C., “High-throughput screening”, Drug Discovery Today 5 (5): 212-214 (2000); “Recent patents in high-throughput screening”, Nat. Biotechnol. 18 (7): 797 (2000); White, R. E., “High-throughput screening in drug metabolism and pharmacokinetic support of drug discovery”, Ann. Rev. Pharmacol. Toxicol. 40: 133-157 (2000); Broach, J. R. and Thomer, J., “High-throughput screening for drug discovery”, Nature 384 (Suppl): 14-16 (1996); Silverman, L. et al., “New assay technologies for high-throughput screening”, Curr. Opin. Chem. Biol. 2:397-403 (1998). Such biochemical assays are based on the activity of the expression product and include standard kinase assays, phosphatase assays, binding assays, assays for apoptosis, hydroxylation, oxidation, conjugation and other enzyme reactions, and assays for protein-protein or protein-DNA or RNA interactions. Cell-based screening assays utilize recombinant host cells expressing the differentially expressed gene product. The recombinant host cells are screened to identify compounds that can activate the product of the differentially expressed gene or increase expression of the gene (i.e. agonists), or inactivate the product of the differentially expressed gene or decrease expression of the gene (i.e. antagonists).

Any of the drug resistance modifying functions mediated by the product of the differentially expressed gene may be used as an endpoint in the screening assay for identifying therapeutic compounds for the treatment of leukemia. See for example, Evans and Guy (2004) Nat. Genet. 236:214-5. Such endpoint assays include assays for cell proliferation, assays for modulation of the cell cycle, assays for the expression of markers indicative of leukemia, and assays for the expression level of genes differentially expressed in leukemia risk groups as described above.

Modulators of the activity of a product of a differentially-expressed gene identified according to these drug screening assays provided above can be used to treat a subject with drug resistant leukemia. These methods of treatment include the steps of administering the modulators of the activity of a product of a differentially-expressed gene in a pharmaceutical composition as described herein, to a subject in need of such treatment.

The following examples are offered by way of illustration and are not intended to be limiting.

### EXAMPLES

### Genomic Determinants Of Cellular Drug Resistance And Treatment Response In Acute Lymphoblastic Leukemia

### I. Introduction

The present study was undertaken to identify genes that are differentially expressed in primary acute lymphoblastic leukemia cells that are sensitive or resistant to the widely used antileukemic agents: prednisolone, vincristine, L-asparaginase and daunorubicin, and to determine whether differential expression of these drug resistance genes influences treatment response. This study has revealed novel patterns of gene expression that confer cellular drug resistance and discriminate treatment outcome.

### II. Methods

### A. Patients and Isolation of Leukemia Cells.

Pre-treatment bone marrow and peripheral blood were obtained after informed consent from children with newly diagnosed acute lymphoblastic leukemia who were enrolled on the ALL-IX Dutch Childhood Leukemia Study Group protocol at the ErasmusMC/Sophia Children's Hospital or on German Cooperative Study Group for Childhood Acute Lymphoblastic Leukemia COALL-92 and 97 treatment protocols (Harms et al. (2003) Blood 102:2736-2740). Mononuclear cells were isolated by sucrose density gradient centrifugation (Lymphoprep™, density 1.077 mg/ml; Nycomed Pharma), within 24 hours after sampling. Cells were re-suspended in culture medium consisting of RPMI 1640 (Dutch modification without L-glutamine; Gibco™) supplemented with 20 percent fetal calf serum (Integro), 2 mM L-glutamine, 200 μg/ml gentamycin (Gibco™) 100 IU/ml penicillin, 100 μg/ml streptomycin, 0.125 μg/ml fungizone (Gibco™), and 5 μg/ml insulin, 5 μg/ml transferrin and 5 ng/ml sodium selenite (ITS media supplement; Sigma-Aldrich Chemie B.V.). Where necessary, leukemic samples were further enriched to more than 90 percent leukemic blasts by removing non-malignant cells with immunomagnetic beads (DynaBeads®). The independent test set consists of patients with acute lymphoblastic leukemia treated on the St. Jude Children's Research Hospital Protocols Total Therapy XIIIA and B. Pui et al. (2003) JAMA 290:2001-7.

### B. In Vitro Drug Resistance Assay.

Sensitivity of leukemia cells to prednisolone (Bufa Pharmaceutical Products), vincristine (TEVA Pharma), L-asparaginase (Paronal, Christiaens), and daunorubicin (Cerubidine, Rhône-Poulenc Rorer) was determined using the 4-day in vitro MTT drug resistance assay, as described in den Boer et al. (2003). J. Clin. Oncol. 21:3262-68. The ranges of concentrations tested were: prednisolone, 0.008-250 μg/ml; vincristine, 0.05-50 μg/ml; L-asparaginase, 0.003-10 IU/ml and daunorubicin, 0.002-2.0 μg/ml. The drug concentration lethal to 50 percent of the leukemia cells (LC50-value) was used as the measure of cellular drug resistance. The LC50-values used to assign cases as sensitive or resistant to each agent, were those previously associated with a good or bad treatment outcome in children with acute lymphoblastic leukemia. See, Table 1.

TABLE 1
LC₅₀ values for classification of resistant and sensitive ALL for each
chemotherapeutic agent*

| Drug | Sensitive | Resistant |
|---|---|---|
| Prednisolone | ≦0.100 μg/ml | ≧150 μg/ml |
| Vincristine | ≦0.391 μg/ml | ≧1.758 μg/ml |
| L-asparaginase | ≦0.033 IU/ml | ≧0.912 IU/ml |
| Daunorubicin | ≦0.075 μg/ml | ≧0.114 μg/ml |

*LC₅₀ by MTT as described by Pieters et al. (1991) Lancet 991:338:399-403. Classification based on LC₅₀ values previously associated with treatment outcome as described in den Boer et al. (2003) J. Clin. Oncol. 21: 3262-68.

### C. RNA Purification, Labeling and Hybridization.

Total cellular RNA was extracted from a minimum of 5×106 leukemic cells using Trizol® reagent (Gibco™), RNA was additionally purified with phenol/chloroform/isoamylalcohol (25:24:1) and RNA integrity was assessed as described in Cheok et al. (2003) Nat. Genet. 34:85-90; and Yeoh et al. (2002) Cancer Cell 1:133-43. RNA processing and hybridization to the U133A GeneChip® oligonucleotide microarray (Affymetrix®) was performed according to manufacturer's protocol.

### D. Data Analysis.

Gene expression values were calculated using Affymetrix® Microarray Suite (MAS) 5.0. 20,21. Expression signals were scaled to the target intensity of 2500 and log-transformed. Arrays were omitted if the scaling factor exceeded three standard deviations of the mean or if either the beta-actin or glyceraldehyde-3-phosphate dehydrogenase (GAPDH) 3′/5′ ratio was greater than three. From the total of 22,283 probe sets, those expressed in fewer than five patients were omitted, leaving 14,550 probe sets for subsequent analyses.

For each antileukemic agent, a significant number of genes that were most discriminative for resistant and sensitive leukemia samples were identified. A Wilcoxon rank sum test and t-test was applied for each probe set and the significance and false discovery rate was estimated using an empirical Bayesian approach, based on one thousand random permutations.

To determine the prediction accuracy using the top discriminating genes, the 173 acute lymphoblastic leukemia patients under study were randomly split into two groups, i.e. two thirds of the patients were used to build the model and the remaining one third to test the accuracy of the model. Prediction accuracy for each antileukemic agent and their confidence intervals were computed based on one thousand random splits using support vector machine as the classifier. In each random split, a gene expression score was assigned to each case in the test set (i.e., 1 if predicted to be sensitive, 2 if predicted to be resistant). The average gene expression score was computed for each patient for all four drugs using top 30, 50, and 100 gene probe sets. The combined drug resistance gene expression score for each patient was calculated as the sum of scores for each individual drug.

Gene expression scores used in the outcome analysis for the 173 Dutch and COALL patients and for the 98 patients 24 in the independent test set were also computed based on only the 172 gene probe sets discriminating sensitive versus resistant leukemia for each drug in the original cohort of patients, utilizing bootstrapping and support vector machine. For the analysis of disease-free survival, any type of leukemia relapse was considered. The duration of disease-free survival was defined as the time from diagnosis until the date of treatment failure. Time was censored at the last follow-up date if no failure was observed. Cox proportional hazard regression analysis was used to assess the association between combined gene expression score and treatment outcome. Leukemia-free survival was analyzed using Fine and Gray's estimator accounting for competing events.

Fisher's exact test was used to determine the over- or under-representation of discriminating genes in specific functional groups compared to the genes present on the U133A GeneChip®, using the Gene Ontology database (www.geneontology.org).

### III. Results

Gene expression was determined in acute lymphoblastic leukemia cells from 173 newly diagnosed patients whose leukemia cells exhibited de novo sensitivity or resistance to a panel of four antileukemic agents, (i.e., prednisolone, vincristine, L-asparaginase and daunorubicin), as assessed in the in vitro MTT assay. The distribution of LC50-values in our study population did not differ from the entire population of 700 patients for whom we had determined sensitivity to each of these antileukemic agents. Likewise, the proportion of patients classified as “sensitive” or “resistant”, using previously defined LC50-values (Table 1) did not differ between the study group and the entire population.

### A. Identification of Differentially Expressed Genes Using Supervised Learning Methods and Assessment of Prediction Accuracy.

Unsupervised hierarchical clustering, which groups patients based on predominant similarities in gene expression, did not cluster patients according to their resistance to any of the four antileukemic agents. Acute lymphoblastic leukemia patients were clustered predominately by immunophenotype. Because T-lineage acute lymphoblastic leukemia cases display a strong gene expression signature, subsequent analyses were performed using either (a) all samples or (b) only the B-lineage acute lymphoblastic leukemia samples. Analyses using only T-lineage ALL patients were not performed because the number of T-ALL cases was too small (n=28).

Supervised methods (i.e., Wilcoxon rank sum test and t-test) were used to build a gene-expression-based discrimination model to identify genes associated with either drug resistance or sensitivity. Selection of genes using either Wilcoxon rank sum test or t-test yielded similar results. Probe sets were rank-ordered according to their P-values, with the smallest P-values indicating the strongest statistical difference between resistant and sensitive patients. Permutation analyses of gene probe sets associated with resistance to prednisolone, vincristine and L-asparaginase gave a high overall significance (P<0.001) in both the total population and within the B-lineage group (Table 2), whereas gene probe sets associated with daunorubicin resistance were significant (P=0.001) in the B-lineage group, but not at the P=0.05 level in the total group. In concordance, the false discovery rate was higher in daunorubicin compared to the other three drugs. For all drugs, the false discovery rates were lower in the B-lineage group compared to the total group (Table 2). Using the top 30, 50 and 100 discriminating genes for each drug, prediction accuracies were 67 to 73 percent, with P-values of 0.007 to 0.045 (Table 3). Within the cohort of patients with B-lineage acute lymphoblastic leukemia, the estimated prediction accuracies were even higher, ranging from 71 to 76 percent, with P-values ranging from 0.004 to 0.025. Multiple logistic regression analysis indicated that gene expression profiles were a significant predictor of drug resistance for all four drugs, independent of known prognostic factors (i.e., age and white blood cell count; Table 4).

TABLE 2a
Patient Permutation analysis and false discovery rate: All patients*

| | α = 0.0001 | | | α = 0.0005 | | | α = 0.001 | | |
|---|---|---|---|---|---|---|---|---|---|
| | | | P- | | | P- | | | P- |
| Drug | n | FDR (%) | value | n | FDR (%) | value | N | FDR (%) | value |
| PRED | 11 | 10 | <0.001 | 32 | 15 | <0.001 | 53 | 22 | <0.001 |
| VCR | 7 | 12 | <0.001 | 76 | 14 | <0.001 | 76 | 14 | <0.001 |
| ASP | 24 | 5 | <0.001 | 91 | 6 | <0.001 | 135 | 9 | <0.001 |
| DNR | 2 | 42 | 0.06 | 11 | 49 | 0.1 | 27 | 44 | 0.07 |

TABLE 2b
Patient Permutation analysis and false discovery rate: B-lineage patients*

| | α = 0.0001 | | | α = 0.0005 | | | α = 0.001 | | |
|---|---|---|---|---|---|---|---|---|---|
| | | | P- | | | P- | | | P- |
| Drug | n | FDR (%) | value | n | FDR (%) | value | N | FDR (%) | value |
| PRED | 19 | 6 | <0.001 | 57 | 8 | <0.001 | 92 | 13 | <0.001 |
| VCR | 22 | 5 | <0.001 | 74 | 8 | <0.001 | 138 | 10 | <0.001 |
| ASP | 67 | 2 | <0.001 | 202 | 3 | <0.001 | 279 | 4 | <0.001 |
| DNR | 5 | 15 | <0.001 | 25 | 21 | 0.001 | 38 | 32 | 0.001 |

*Permutation analysis (n = 1000) was computed for each dataset (prednisolone (PRED), vincristine (VCR), L-asparaginase (ASP), daunorubicin (DNR) using (a) all patients and (b) only patients with B-lineage acute lymphoblastic leukemia. For each P-value using Wilcoxon rank sum rank test (α), the number of probe sets (n), the false discovery rate (FDR) and the overall significance (P-value) are listed. In each random permutation, the class label (resistant or sensitive) was randomly assigned to each patient and genes were reselected using Wilcoxon rank sum test and t-test based on the random labels. The overall significance (P α) of the model was estimated using the following formula: P_{α = (n permutations with N}_{α}^{random} ≧ N_{α}^{obs})/(Total n of permutations)
Where α is the P-value using Wilcoxon rank sum test and t-test; N_{α}^{random} is the number of probe sets with P-values less than a using random class label; N_{α}^{obs} is the number of probe sets with P-values less than α using the observed class label. The false discovery rate (FDR_{α} was estimated as: FDR_{α} = (median(N_{α}^{random}))N_{α}^{obs}
Principal component analysis and 2D-hierarchical clustering were performed using GeneMaths ™ 2.1 software (AppliedMaths, St. Martens-Latem, Belgium). To show that discriminating genes were not obtained by chance, the significance and false discovery rate was estimated using an empirical Bayesian approach based on one thousand permutations. The top principal components based on top-ranked probe sets (30, 50 or 100) re-selected by Wilcoxon rank sum test were used to construct support vector machines as prediction models. Statistical significance of the prediction accuracy compared to chance (50 percent accuracy) was determined by permutation analyses.

TABLE 3a
Patient prediction accuracy using gene expression profiles for
classification of drug resistant and sensitive acute lymphoblastic leukemia:
All patients*

| | n | accuracy (%) | 95% C.I. | P-value |
|---|---|---|---|---|
| PRED | 30 | 71 | 58-81 | 0.031 |
| | 50 | 71 | 58-81 | 0.031 |
| | 100 | 71 | 58-81 | 0.031 |
| VCR | 30 | 68 | 59-78 | 0.029 |
| | 50 | 71 | 59-79 | 0.012 |
| | 100 | 71 | 59-79 | 0.012 |
| ASP | 30 | 67 | 53-76 | 0.045 |
| | 50 | 67 | 52-79 | 0.045 |
| | 100 | 67 | 55-78 | 0.045 |
| DNR | 30 | 73 | 60-80 | 0.007 |
| | 50 | 73 | 60-80 | 0.007 |
| | 100 | 73 | 58-76 | 0.007 |

TABLE 3b
Patient prediction accuracy using gene expression profiles for
classification of drug resistant and sensitive acute lymphoblastic leukemia:
B-lineage patients*

| | n | accuracy (%) | 95% C.I. | P-value |
|---|---|---|---|---|
| PRED | 30 | 75 | 63-88 | 0.025 |
| | 50 | 75 | 63-88 | 0.025 |
| | 100 | 75 | 67-87 | 0.025 |
| VCR | 30 | 74 | 62-85 | 0.010 |
| | 50 | 76 | 62-85 | 0.004 |
| | 100 | 76 | 62-85 | 0.004 |
| ASP | 30 | 71 | 60-83 | 0.018 |
| | 50 | 71 | 57-83 | 0.018 |
| | 100 | 71 | 60-80 | 0.019 |
| DNR | 30 | 76 | 65-85 | 0.004 |
| | 50 | 76 | 65-85 | 0.004 |
| | 100 | 76 | 67-84 | 0.004 |

*Patient Prediction accuracy using gene expression profiles for classification of drug resistant and sensitive acute lymphoblastic leukemia. Prediction accuracy for each antileukemic agent using 30, 50 or 100 probe sets. The median prediction accuracy is shown with corresponding P-values and the 95 percent confidence interval (C.I.) for prednisolone (PRED), vincristine (VCR), L-asparaginase (ASP) and daunorubicin (DNR) (a) based on all patients and (b) for only patients with B-lineage acute lymphoblastic leukemia.

TABLE 4
Multivariate analysis of gene expression and known prognostic
factors (age, WBC count) to discriminate drug resistance*

| | | Odds ratio | | |
|---|---|---|---|---|
| | Predictor | (95% C.I.) | | P-value |
| PRED | gene expression score | 57.8 | (4.9-681.6) | 0.001 |
| | age (years) | 1.17 | (1.01-1.36) | 0.038 |
| | WBC count (10⁹/L) | 1.0 | (0.98-1.01) | 0.427 |
| VCR | gene expression score | 13.5 | (1.97-92.6) | 0.008 |
| | age (years) | 1.0 | (0.89-1.13) | 0.995 |
| | WBC count (10⁹/L) | 0.99 | (0.99-1.0) | 0.084 |
| ASP | gene expression score | 22.1 | (4.8-102.6) | <0.001 |
| | age (years) | 1.13 | (1.01-1.26) | 0.033 |
| | WBC count (10⁹/L) | 1.0 | (0.99-1.01) | 0.792 |
| DNR | gene expression score | 151.2 | (4.1-5533.6) | 0.006 |
| | age (years) | 1.04 | (0.93-1.17) | 0.455 |
| | WBC count (10⁹/L) | 1.0 | (0.99-1.0) | 0.273 |

*Multiple logistic regression was used with gene expression and known prognostic factors (age, WBC count) to discriminate drug resistance.

Gene expression scores were also computed for the patients with intermediate drug sensitivity (Table 5), revealing median scores that were between the median gene expression score of the drug sensitive and drug resistant groups for all four antileukemic agents. For L-asparaginase and prednisolone, the gene expression scores of the intermediate group were significantly different from both the sensitive group and the resistant group (P<0.05, Wilcoxon rank sum test). For daunorubicin and vincristine, the intermediate group was significantly different from the sensitive group, but not from the resistant group (Table 5).

TABLE 5
Gene expression scores for the intermediate sensitivity group, using
genes selected to discriminate resistant and sensitive B-lineage ALL*

| | | resistant | intermediate | | | | |
|---|---|---|---|---|---|---|---|
| | n | (R) | (I) | sensitive (S) | R vs. S | R vs. I | I vs. S |
| PRED | 50 | 1.28 | 1.04 | 1.02 | <0.001 | 0.002 | 0.044 |
| | | (1.01-1.96) | (1.0-1.88) | (1.0-1.65) | | | |
| | 100 | 1.29 | 1.01 | 1.01 | <0.001 | 0.001 | 0.035 |
| | | (1.01-1.99) | (1.0-1.92) | (1.0-1.72) | | | |
| VCR | 50 | 1.29 | 1.29 | 1.11 | 0.006 | 0.659 | 0.002 |
| | | (1.01-1.88) | (1.01-1.61) | (1.01-1.9) | | | |
| | 100 | 1.26 | 1.24 | 1.08 | 0.002 | 0.518 | 0.002 |
| | | (1.01-1.93) | (1.0-1.68) | (1.0-1.94) | | | |
| ASP | 50 | 1.53 | 1.31 | 1.14 | <0.001 | 0.013 | 0.004 |
| | | (1.03-1.91) | (1.02-1.88) | (1.01-1.97) | | | |
| | 100 | 1.51 | 1.31 | 1.1 | <0.001 | 0.024 | 0.008 |
| | | (1.02-1.91) | (1.0-1.92) | (1.0-1.98) | | | |
| DNR | 50 | 1.16 | 1.11 | 1.04 | 0.021 | 0.404 | 0.090 |
| | | (1.0-1.5) | (1.0-1.71) | (1.0-1.6) | | | |
| | 100 | 1.15 | 1.07 | 1.01 | 0.019 | 0.583 | 0.045 |
| | | (1.0-1.47) | (1.0-1.81) | (1.0-1.56) | | | |

*Gene expression scores for the intermediate sensitivity group, using genes selected to discriminate resistant and sensitive B-lineage ALL. The resistant and sensitive patients were randomly split into ⅔ training set and ⅓ test set. Additionally, all intermediate patients were included in the test set. A patient in the test set was assigned a score of 1 if classified by the model as sensitive and 2 if resistant; no score was assigned to patients in the training set. The above was repeated 1000 times to compute a gene expression score for each patient as the average of scores ever assigned to the patient. Median gene expression scores of resistant (R), sensitive (S) and intermediate (I) samples as well as the range in parentheses are presented based on 50 and 100 probe sets. The P-values are given for each pair wise comparison, using Wilcoxon rank sum test.

### B. Supervised Clustering and Principal Component Analysis.

The number of genes used to build the drug resistance model for each antileukemic agent was determined based upon the false discovery rate, permutation analysis and prediction accuracy for all patients (Table 2). This identified 172 probe sets corresponding to 123 unique gene annotations and 30 cDNA clones (some genes are represented on the array by multiple probe sets), that were differentially expressed in sensitive and resistant B-lineage acute lymphoblastic leukemia. Hierarchical clustering using the selected probe sets correctly assigned 66 of 74 cases for prednisolone (89 percent apparent accuracy), 84 of 104 for vincristine (81 percent), 83 of 106 for L-asparaginase (78 percent) and 86 of 105 for daunorubicin (82 percent). Similarly, principal component analyses correctly grouped the majority of patients into either the resistant cluster or the sensitive cluster for each of the four antileukemic agents. Hierarchical clustering and principal component analyses of all patients gave similar results. The probe set ID, gene names, annotations and the gene expression ratio for resistant versus sensitive leukemia for discriminating genes are shown for each drug in Tables 6-9 (B-lineage acute lymphoblastic leukemia) and Tables 10-13 (B- and T-lineage acute lymphoblastic leukemia).

TABLE 6A
Top genes discriminating prednisolone resistant and sensitive B-
lineage ALL: Genes down-regulated in prednisolone resistant B-lineage
ALL

| | | | | NCBI |
|---|---|---|---|---|
| | | | R/S | Accession |
| Probe ID | Gene Name | Gene Symbol | ratio | Number |
| 208660_at | citrate synthase | CS | 0.82 | BC000105 |
| 201896_s_at | CDC28 protein kinase | CKS1B | 0.39 | BC001425 |
| | regulatory subunit 1B | | | |
| 209675_s_at | E1B-55 kDa-associated protein 5 | E1B-AP5 | 0.70 | BC004242 |
| 217729_s_at | amino-terminal enhancer | AES | 0.60 | NM_001130 |
| | of split | | | |
| 209760_at | KIAA0922 protein | KIAA0922 | 0.65 | AL136932 |
| 202521_at | CCCTC-binding factor | CTCF | 0.67 | NM_006565 |
| | (zinc finger protein) | | | |
| 212167_s_at | SWI/SNF related, matrix | SMARCB1 | 0.76 | AK021419 |
| | associated, actin | | | |
| | dependent regulator | | | |
| 201938_at | CDK2-associated protein 1 | CDK2AP1 | 0.47 | NM_004642 |
| 216484_x_at | hepatoma-derived | HDGF | 0.71 | L24521 |
| | growth factor (high- | | | |
| | mobility group protein 1-1 | | | |
| 200896_x_at | hepatoma-derived | HDGF | 0.70 | NM_004494 |
| | growth factor (high- | | | |
| | mobility group protein 1-1 | | | |
| 38710_at | ubiquitin-specific | FLJ20113 | 0.79 | AL096714 |
| | protease otubain 1 | | | |
| 212100_s_at | KIAA1649 protein | KIAA1649 | 0.71 | Z93241 |
| 219679_s_at | WW domain-containing | WAC | 0.69 | NM_018604 |
| | adapter with a coiled-coil | | | |
| | region | | | |
| 221547_at | PRP18 pre-mRNA | PRPF18 | 0.72 | BC000794 |
| | processing factor 18 | | | |
| | homolog (yeast) | | | |
| 217978_s_at | NICE-5 protein | HSA243666 | 0.77 | NM_017582 |
| 213061_s_at | hypothetical protein | LOC123803 | 0.59 | AA643304 |
| | LOC123803 | | | |
| 208766_s_at | heterogeneous nuclear | R HNRPR | 0.82 | BC001449 |
| | ribonucleoprotein | | | |
| 212910_at | HRIHFB2206 protein | HRIHFB2206 | 0.54 | W19873 |
| 47083_at | hypothetical protein | MGC2718 | 0.81 | AI280108 |
| | MGC2718 | | | |
| 208781_x_at | sorting nexin 3 | SNX3 | 0.77 | AF062483 |
| 218438_s_at | endothelial-derived gene 1 | EG1 | 0.76 | NM_025205 |
| 208620_at | poly(rC) binding protein 1 | PCBP1 | 0.67 | U24223 |
| 203274_at | coagulation factor VIII- | F8A | 0.51 | NM_012151 |
| | associated (intronic | | | |
| | transcript) | | | |
| 208739_x_at | SMT3 suppressor of mif | SMT3H2 | 0.69 | L76416 |
| | two 3 homolog 2 (yeast) | | | |

TABLE 6B
Top genes discriminating prednisolone resistant and sensitive B-
lineage ALL: Genes up-regulated in prednisolone resistant ALL

| | | | | NCBI |
|---|---|---|---|---|
| | | | R/S | Accession |
| Probe ID | Gene Name | Gene Symbol | ratio | Number |
| 206209_s_at | carbonic anhydrase IV | CA4 | 1.56 | NM_000717 |
| 208205_at | protocadherin alpha 9 | PCDHA9 | 1.63 | NM_014005 |
| 206905_s_at | matrilin 1, cartilage matrix protein | MATN1 | 2.04 | NM_002379 |
| 207452_s_at | contactin 5 | CNTN5 | 1.58 | NM_014361 |
| 212581_x_at | glyceraldehyde-3- | GAPD | 1.25 | BE561479 |
| | phosphate | | | |
| | dehydrogenase | | | |
| 217398_x_at | glyceraldehyde-3- | GAPD | 1.37 | AK026525 |
| | phosphate | | | |
| | dehydrogenase | | | |
| AFFX- | glyceraldehyde-3- | GAPD | 1.21 | M33197 |
| HUMGAP | phosphate | | | |
| | dehydrogenase | | | |
| AFFX- | glyceraldehyde-3- | GAPD | 1.20 | M33197 |
| HUMGAP | phosphate | | | |
| | dehydrogenase | | | |
| 213453_x_at | glyceraldehyde-3- | GAPD | 1.26 | BF689355 |
| | phosphate | | | |
| | dehydrogenase | | | |
| 214057_at | myeloid cell leukemia | MCL1 | 1.72 | H71805 |
| | sequence 1 (BCL2- | | | |
| | related) | | | |
| 203149_at | poliovirus receptor- | PVRL2 | 2.01 | NM_002856 |
| | related 2 (herpesvirus | | | |
| | entry mediator B) | | | |
| 222088_s_at | solute carrier family 2 | SLC2A14 | 2.07 | AA778684 |
| | (facilitated glucose | | | |
| | transporter) | | | |
| 205193_at | v-maf | MAFF | 1.69 | NM_012323 |
| | musculoaponeurotic | | | |
| | fibrosarcoma oncogene | | | |
| | homolog F | | | |
| 36711_at | v-maf | MAFF | 2.06 | AL021977 |
| | musculoaponeurotic | | | |
| | fibrosarcoma oncogene | | | |
| | homolog F | | | |
| 202201_at | biliverdin reductase B | BLVRB | 2.31 | NM_000713 |
| | (flavin reductase | | | |
| | (NADPH)) | | | |
| 201061_s_at | stomatin | STOM | 1.84 | M81635 |
| 218589_at | purinergic receptor | P2RY5 | 2.91 | NM_005767 |
| | P2Y, G-protein | | | |
| | coupled, 5 | | | |
| 209795_at | CD69 antigen (p60, | CD69 | 2.26 | L07555 |
| | early T-cell activation | | | |
| | antigen) | | | |

TABLE 6C
Genes discriminating prednisolone resistant and sensitive B-
lineage ALL to the 0.001 < p < 0.01 level by both T-test and Wilcoxon: Genes
down-regulated in prednisolone resistant B-lineage ALL

| NCBI Accession | | Gene |
|---|---|---|
| Number | Gene Name | Symbol |
| NM_013299 | protein predicted by clone 23627 | HSU79266 |
| U84138 | RAD51-like 1 (S. cerevisiae) | RAD51L1 |
| NM_005679 | “TATA box binding protein (TBP)-associated | TAF1C |
| | factor, RNA polymerase I, C, 110 kDa” | |
| AL582836 | paternally expressed 10 | PEG10 |
| AF277194 | putative membrane protein | LOC54499 |
| BC000229 | hypothetical protein MGC2488 | MGC2488 |
| NM_022149 | MAGEF1 protein | MAGEF1 |
| AL536319 | KIAA0993 protein | KIAA0993 |
| NM_004190 | “lipase, gastric” | LIPF |
| NM_014292 | chromobox homolog 6 | CBX6 |
| BC001425 | p53-regulated DDA3 | DDA3 |
| NM_001431 | erythrocyte membrane protein band 4.1-like 2 | EPB41L2 |
| AF068220 | “ATPase, Ca++ transporting, ubiquitous” | ATP2A3 |
| NM_016553 | nucleoporin 62 kDa | NUP62 |
| AW299740 | dihydrolipoamide S-acetyltransferase (E2 | DLAT |
| | component of pyruvate dehydrogenase | |
| | complex) | |
| U49396 | “purinergic receptor P2X, ligand-gated ion | P2RX5 |
| | channel, 5” | |
| NM_000465 | BRCA1 associated RING domain 1 | BARD1 |
| AK001327 | Tax interaction protein 1 | TIP-1 |
| NM_018036 | hypothetical protein FLJ10242 | FLJ10242 |
| NM_016256 | N-acetylglucosamine-1-phosphodiester alpha- | LOC51172 |
| | N-acetylglucosaminidase | |
| NM_006806 | “BTG family, member 3” | BTG3 |
| AF257659 | calumenin | CALU |
| NM_017920 | up-regulated gene 4 | URG4 |
| NM_002383 | MYC-associated zinc finger protein (purine- | MAZ |
| | binding transcription factor) | |
| NM_021212 | HCF-binding transcription factor Zhangfei | ZF |
| AK026607 | p53-induced protein | PIG11 |
| NM_014345 | endocrine regulator | HRIHFB2436 |
| AW450363 | ADP-ribosylation factor-like 7 | ARL7 |
| AL110206 | Homo sapiens mRNA; cDNA | |
| | DKFZp586N2022 (from clone | |
| | DKFZp586N2022) | |
| BF677486 | “thymosin, beta, identified in neuroblastoma | TMSNB |
| | cells” | |
| AW138827 | “TAF5 RNA polymerase II, TATA box | TAF5 |
| | binding protein (TBP)-associated factor, | |
| | 100 kDa” | |
| NM_024067 | hypothetical protein MGC2718 | MGC2718 |
| BF576458 | nuclear receptor coactivator 1 | NCOA1 |
| BE646618 | mitogen-activated protein kinase kinase | MAP4K1 |
| | kinase kinase 1 | |
| AK026682 | “Homo sapiens cDNA: FLJ23029 fis, clone | |
| | LNG01883” | |
| NM_004053 | bystin-like | BYSL |
| NM_018095 | hypothetical protein FLJ10450 | FLJ10450 |
| W19873 | HRIHFB2206 protein | HRIHFB2206 |
| X17115 | immunoglobulin heavy constant mu | IGHM |
| AI858004 | immunoglobulin heavy constant gamma 3 | IGHG3 |
| | (G3m marker) | |
| AF312918 | Mlx interactor | MONDOA |
| AW270932 | hypothetical protein FLJ20275 | FLJ20275 |
| AI800983 | “cleavage and polyadenylation specific factor | CPSF5 |
| | 5, 25 kDa” | |
| NM_025104 | Dbf4-related factor 1 | DRF1 |
| NM_004642 | CDK2-associated protein 1 | CDK2AP1 |
| NM_014736 | KIAA0101 gene product | KIAA0101 |
| NM_014322 | “opsin 3 (encephalopsin, panopsin)” | OPN3 |
| AA877910 | “ATPase, Ca++ transporting, ubiquitous” | ATP2A3 |
| NM_021992 | “thymosin, beta, identified in neuroblastoma | TMSNB |
| | cells” | |
| NM_012151 | coagulation factor VIII-associated (intronic | F8A |
| | transcript) | |
| NM_000626 | CD79B antigen (immunoglobulin-associated | CD79B |
| | beta) | |
| NM_014791 | maternal embryonic leucine zipper kinase | MELK |
| BF510692 | paired box gene 5 (B-cell lineage specific | PAX5 |
| | activator protein) | |
| AA643304 | Homo sapiens mRNA; cDNA | |
| | DKFZp666E058 (from clone | |
| | DKFZp666E058) | |
| BG391171 | thymopoietin | TMPO |
| NM_002358 | MAD2 mitotic arrest deficient-like 1 (yeast) | MAD2L1 |
| AV682679 | selenium donor protein | SPS |
| J04977 | “X-ray repair complementing defective repair | XRCC5 |
| | in Chinese hamster cells 5 (double-strand- | |
| | break rejoining; Ku autoantigen, 80 kDa)” | |
| AB014562 | KIAA0662 gene product | KIAA0662 |
| M18003 | ferredoxin 1 | FDX1 |
| AK000993 | “Homo sapiens cDNA FLJ10131 fis, clone | |
| | HEMBA1003041” | |
| NM_005173 | “ATPase, Ca++ transporting, ubiquitous” | ATP2A3 |
| AF151853 | preimplantation protein 3 | PREI3 |
| NM_021813 | “BTB and CNC homology 1, basic leucine | BACH2 |
| | zipper transcription factor 2” | |
| BF340123 | hypothetical protein FLJ11149 | FLJ11149 |
| NM_004111 | flap structure-specific endonuclease 1 | FEN1 |
| BE964689 | ubiquitin-conjugating enzyme E2L 3 | UBE2L3 |
| BF432873 | “proteasome (prosome, macropain) 26S | PSMD11 |
| | subunit, non-ATPase, 11” | |
| BF034561 | G-rich RNA sequence binding factor 1 | GRSF1 |
| NM_005663 | Wolf-Hirschhorn syndrome candidate 2 | WHSC2 |
| NM_007275 | lung cancer candidate | FUS1 |
| AW272611 | thymopoietin | TMPO |
| NM_017980 | LIM and senescent cell antigen-like domains 2 | LIMS2 |
| AF231056 | “SWI/SNF related, matrix associated, actin | SMARCF1 |
| | dependent regulator of chromatin, subfamily | |
| | f, member 1” | |
| AL137673 | Homo sapiens mRNA; cDNA | |
| | DKFZp434H0872 (from clone | |
| | DKFZp434H0872) | |
| NM_023941 | hypothetical protein MGC3032 | MGC3032 |
| NM_004450 | enhancer of rudimentary homolog | ERH |
| | (Drosophila) | |
| NM_002466 | v-myb myeloblastosis viral oncogene | MYBL2 |
| | homolog (avian)-like 2 | |
| BF001666 | Homo sapiens clone 23870 mRNA sequence | |
| NM_001938 | “down-regulator of transcription 1, TBP- | DR1 |
| | binding (negative cofactor 2)” | |
| BC004439 | translocase of inner mitochondrial membrane | TIMM17A |
| | 17 homolog A (yeast) | |
| Y15724 | “ATPase, Ca++ transporting, ubiquitous” | ATP2A3 |
| NM_001130 | amino-terminal enhancer of split | AES |
| AW612574 | lecuine-rich acidic protein-like protein | LANP-L |
| NM_016200 | U6 snRNA-associated Sm-like protein LSm8 | LOC51691 |
| BG338532 | SMT3 suppressor of mif two 3 homolog 1 | SMT3H1 |
| | (yeast) | |
| AB011093 | Rho-specific guanine nucleotide exchange | P114-RHO- |
| | factor p114 | GEF |
| AB018305 | “spondin 1, (f-spondin) extracellular matrix | SPON1 |
| | protein” | |
| AF035737 | “general transcription factor II, i” | GTF2I |
| AL515874 | “Homo sapiens cDNA FLJ33806 fis, clone | |
| | CTONG2000846” | |
| NM_021940 | stromal membrane-associated protein | SMAP1 |
| NM_003372 | von Hippel-Lindau binding protein 1 | VBP1 |
| AA195999 | mitogen-activated protein kinase 1 | MAPK1 |
| NM_024649 | hypothetical protein FLJ23590 | FLJ23590 |
| NM_003685 | KH-type splicing regulatory protein (FUSE | KHSRP |
| | binding protein 2) | |
| AL136932 | KIAA0922 protein | KIAA0922 |
| BC004273 | “splicing factor 3b, subunit 4, 49 kDa” | SF3B4 |
| BG528818 | pre-mRNA splicing factor 17 | PRP17 |
| AF267865 | DKFZP564A043 protein | DKFZP564A043 |
| NM_002979 | sterol carrier protein 2 | SCP2 |
| NM_006577 | “UDP-GlcNAc:betaGal beta-1,3-N- | B3GNT1 |
| | acetylglucosaminyltransferase 1” | |
| NM_001783 | CD79A antigen (immunoglobulin-associated | CD79A |
| | alpha) | |
| AI936769 | “FK506 binding protein 1A, 12 kDa” | FKBP1A |
| L21990 | “splicing factor 3a, subunit 2, 66 kDa” | SF3A2 |
| U76248 | seven in absentia homolog 2 (Drosophila) | SIAH2 |
| AF101434 | Wolf-Hirschhorn syndrome candidate 2 | WHSC2 |
| NM_006565 | CCCTC-binding factor (zinc finger protein) | CTCF |
| AF217963 | “melanoma antigen, family D, 1” | MAGED1 |
| AV756141 | “colony stimulating factor 2 receptor, beta, | CSF2RB |
| | low-affinity (granulocyte-macrophage)” | |
| AF234997 | Tax interaction protein 1 | TIP-1 |
| NM_012394 | prefoldin 2 | PFDN2 |
| NM_018604 | WW domain-containing adapter with a | WAC |
| | coiled-coil region | |
| M19156 | keratin 10 (epidermolytic hyperkeratosis; | KRT10 |
| | keratosis palmaris et plantaris) | |
| AI478300 | “ESTs, Weakly similar to neuronal thread | |
| | protein [Homo sapiens] [H. sapiens]” | |
| NM_015895 | “geminin, DNA replication inhibitor” | GMNN |
| U20498 | “cyclin-dependent kinase inhibitor 2D (p19, | CDKN2D |
| | inhibits CDK4)” | |
| Z25435 | | |
| BC005338 | “capping protein (actin filament) muscle Z- | CAPZA2 |
| | line, alpha 2” | |
| BC003376 | “ELAV (embryonic lethal, abnormal vision, | ELAVL1 |
| | Drosophila)-like 1 (Hu antigen R)” | |
| NM_004759 | mitogen-activated protein kinase-activated | MAPKAPK2 |
| | protein kinase 2 | |
| BC000903 | high-mobility group box 2 | HMGB2 |
| BC004242 | E1B-55 kDa-associated protein 5 | E1B-AP5 |
| NM_013387 | ubiquinol-cytochrome c reductase complex | HSPC051 |
| | (7.2 kD) | |
| NM_000801 | “FK506 binding protein 1A, 12 kDa” | FKBP1A |
| NM_003133 | signal recognition particle 9 kDa | SRP9 |
| NM_024299 | chromosome 20 open reading frame 149 | C20orf149 |
| AI744900 | “SWI/SNF related, matrix associated, actin | SMARCA4 |
| | dependent regulator of chromatin, subfamily | |
| | a, member 4” | |
| NM_024644 | hypothetical protein FLJ21802 | FLJ21802 |
| AI589507 | hypothetical protein MGC5466 | MGC5466 |
| NM_016640 | mitochondrial ribosomal protein S30 | MRPS30 |
| AK026120 | KIAA0460 protein | KIAA0460 |
| NM_015955 | C21orf19-like protein | LOC51072 |
| NM_021183 | “hypothetical protein similar to small G | LOC57826 |
| | proteins, especially RAP-2A” | |
| BC000794 | PRP18 pre-mRNA processing factor 18 | PRPF18 |
| | homolog (yeast) | |
| NM_014045 | low density lipoprotein receptor-related | LRP10 |
| | protein 10 | |
| AK000311 | Homo sapiens mRNA; cDNA | |
| | DKFZp564E2222 (from clone | |
| | DKFZp564E2222) | |
| NM_021183 | “hypothetical protein similar to small G | LOC57826 |
| | proteins, especially RAP-2A” | |
| NM_021242 | hypothetical protein STRAIT11499 | STRAIT11499 |
| M25269 | “ELK1, member of ETS oncogene family” | ELK1 |
| NM_007146 | zinc finger protein 161 | ZNF161 |
| AF067173 | “mago-nashi homolog, proliferation- | MAGOH |
| | associated (Drosophila)” | |
| NM_005826 | heterogeneous nuclear ribonucleoprotein R | HNRPR |
| D26156 | “SWI/SNF related, matrix associated, actin | SMARCA4 |
| | dependent regulator of chromatin, subfamily | |
| | a, member 4” | |
| U24223 | poly(rC) binding protein 1 | PCBP1 |
| BC004913 | papillary renal cell carcinoma (translocation- | PRCC |
| | associated) | |
| NM_006333 | nuclear DNA-binding protein | C1D |
| NM_018221 | chromosome 2 open reading frame 6 | C2orf6 |
| AF141304 | “RAB5C, member RAS oncogene family” | RAB5C |
| NM_014820 | translocase of outer mitochondrial membrane | TOMM70A |
| | 70 homolog A (yeast) | |
| D63882 | “DMC1 dosage suppressor of mck1 homolog, | DMC1 |
| | meiosis-specific homologous recombination | |
| | (yeast)” | |
| NM_014165 | HSPC125 protein | HSPC125 |
| NM_003094 | small nuclear ribonucleoprotein polypeptide E | SNRPE |
| BC002809 | “down-regulator of transcription 1, TBP- | DR1 |
| | binding (negative cofactor 2)” | |
| AL134724 | Homo sapiens clone 24711 mRNA sequence | |
| L24521 | “Human transformation-related protein | |
| | mRNA, 3′ end” | |
| AA586774 | “serine/threonine kinase 24 (STE20 homolog, | STK24 |
| | yeast)” | |
| L20817 | “discoidin domain receptor family, member | DDR1 |
| | 1” | |
| NM_015343 | likely ortholog of Xenopus dullard | HSA011916 |
| U82819 | “uncoupling protein 2 (mitochondrial, proton | UCP2 |
| | carrier)” | |
| NM_002208 | “integrin, alpha E (antigen CD103, human | ITGAE |
| | mucosal lymphocyte antigen 1; alpha | |
| | polypeptide)” | |
| Z93241 | hypothetical protein DKFZP434G0310 | DKFZP434G0310 |
| AI363375 | hypothetical protein BC002926 | LOC90379 |
| NM_004494 | hepatoma-derived growth factor (high- | HDGF |
| | mobility group protein 1-like) | |
| NM_005496 | SMC4 structural maintenance of | SMC4L1 |
| | chromosomes 4-like 1 (yeast) | |
| NM_014628 | gene predicted from cDNA with a complete | KIAA0110 |
| | coding sequence | |
| NM_006402 | hepatitis B virus x interacting protein | HBXIP |
| NM_014837 | chromosome 1 open reading frame 16 | C1orf16 |
| L76416 | SMT3 suppressor of mif two 3 homolog 2 | SMT3H2 |
| | (yeast) | |
| AI741124 | “guanine nucleotide binding protein (G | GNB1 |
| | protein), beta polypeptide 1” | |
| NM_006559 | “KH domain containing, RNA binding, signal | KHDRBS1 |
| | transduction associated 1” | |
| AW516932 | “down-regulator of transcription 1, TBP- | DR1 |
| | binding (negative cofactor 2)” | |
| NM_007373 | soc-2 suppressor of clear homolog (C. elegans) | SHOC2 |
| NM_014403 | “sialyltransferase 7D ((alpha-N- | SIAT7D |
| | acetylneuraminyl-2,3-beta-galactosyl-1,3)-N- | |
| | acetyl galactosaminide alpha-2,6- | |
| | sialyltransferase)” | |
| NM_014225 | “protein phosphatase 2 (formerly 2A), | PPP2R1A |
| | regulatory subunit A (PR 65), alpha isoform” | |
| AD001527 | cytoskeleton-associated protein 1 | CKAP1 |
| NM_006388 | “HIV-1 Tat interactive protein, 60 kDa” | HTATIP |
| BC005147 | “FK506 binding protein 1A, 12 kDa” | FKBP1A |
| NM_025205 | endothelial-derived gene 1 | EG1 |
| BC005212 | hypothetical protein FLJ20003 | FLJ20003 |
| NM_004622 | translin | TSN |
| AI471665 | MYC-associated zinc finger protein (purine- | MAZ |
| | binding transcription factor) | |
| AK021419 | “SWI/SNF related, matrix associated, actin | SMARCB1 |
| | dependent regulator of chromatin, subfamily | |
| | b, member 1” | |
| AK022555 | KIAA0618 gene product | KIAA0618 |
| AA534860 | histone H2A.F/Z variant | H2AV |
| NM_031298 | hypothetical protein MGC2963 | MGC2963 |
| NM_007234 | dynactin 3 (p22) | DCTN3 |
| BC005876 | “ATPase, H+ transporting, lysosomal 21 kDa, | ATP6V0B |
| | V0 subunit c”” | |
| NM_003173 | suppressor of variegation 3-9 homolog 1 | SUV39H1 |
| | (Drosophila) | |
| NM_006826 | “tyrosine 3-monooxygenase/tryptophan 5- | YWHAQ |
| | monooxygenase activation protein, theta | |
| | polypeptide” | |
| NM_007279 | U2 small nuclear ribonucleoprotein auxiliary | U2AF65 |
| | factor (65 kD) | |
| AF067173 | “mago-nashi homolog, proliferation- | MAGOH |
| | associated (Drosophila)” | |
| NM_017582 | NICE-5 protein | HSA243666 |
| BF675004 | “poly(A) binding protein, nuclear 1” | PABPN1 |
| NM_015485 | DKFZP566K023 protein | DKFZP566K023 |
| AF062483 | sorting nexin 3 | SNX3 |
| AI679080 | “SWI/SNF related, matrix associated, actin | SMARCF1 |
| | dependent regulator of chromatin, subfamily | |
| | f, member 1” | |
| NM_003769 | “splicing factor, arginine/serine-rich 9” | SFRS9 |
| NM_002074 | “guanine nucleotide binding protein (G | GNB1 |
| | protein), beta polypeptide 1” | |
| AW139179 | fem-1 homolog b (C. elegans) | FEM1B |
| AL096714 | hypothetical protein FLJ20113 | FLJ20113 |
| AI280108 | hypothetical protein MGC2718 | MGC2718 |
| W93787 | “golgi reassembly stacking protein 2, 55 kDa” | GORASP2 |
| NM_005334 | host cell factor C1 (VP16-accessory protein) | HCFC1 |
| NM_006842 | “splicing factor 3b, subunit 2, 145 kDa” | SF3B2 |
| AL031133 | “ESTs, Highly similar to SM32_HUMAN | |
| | Ubiquitin-like protein SMT3B (Sentrin 2) | |
| | [H. sapiens]” | |
| BE748755 | “chromobox homolog 3 (HP1 gamma | CBX3 |
| | homolog, Drosophila)” | |
| BC004239 | jumping translocation breakpoint | JTB |
| BC000105 | citrate synthase | CS |
| BC001449 | heterogeneous nuclear ribonucleoprotein R | HNRPR |
| AB047360 | sorting nexin 3 | SNX3 |
| NM_005877 | “splicing factor 3a, subunit 1, 120 kDa” | SF3A1 |
| AF135162 | cyclin I | CCNI |
| AF141349 | hypothetical protein DKFZp434N0650 | DKFZp434N0650 |
| NM_006694 | jumping translocation breakpoint | JTB |
| D80006 | disco-interacting protein 2 (Drosophila) | DIP2 |
| | homolog | |
| AW873564 | “ESTs, Weakly similar to YHS1_YEAST | |
| | HYPOTHETICAL 21.3 KD PROTEIN IN | |
| | MSH1-EPT1 INTERGENIC REGION | |
| | [S. cerevisiae]” | |

TABLE 6D
Genes discriminating prednisolone resistant and sensitive B-
lineage ALL to the 0.001 < p < 0.01 level by both T-test and Wilcoxon: Genes
up-regulated in prednisolone resistant B-lineage ALL

| NCBI Accession | | |
|---|---|---|
| Number | Gene Name | Gene Symbol |
| NM_018955 | ubiquitin B | UBB |
| AA349595 | KIAA1091 protein | KIAA1091 |
| NM_017627 | hypothetical protein FLJ20030 | FLJ20030 |
| NM_001006 | ribosomal protein S3A | RPS3A |
| BF184532 | ribosomal protein S20 | RPS20 |
| M33197 | glyceraldehyde-3-phosphate dehydrogenase | GAPD |
| BC001019 | ribosomal protein L39 | RPL39 |
| M33197 | glyceraldehyde-3-phosphate dehydrogenase | GAPD |
| M33197 | glyceraldehyde-3-phosphate dehydrogenase | GAPD |
| AA789278 | ribosomal protein L13 | RPL13 |
| AW574664 | ribosomal protein L13 | RPL13 |
| BE561479 | glyceraldehyde-3-phosphate dehydrogenase | GAPD |
| BF689355 | glyceraldehyde-3-phosphate dehydrogenase | GAPD |
| BC004954 | ribosomal protein L13 | RPL13 |
| NM_004834 | mitogen-activated protein kinase kinase | MAP4K4 |
| | kinase kinase 4 | |
| AI186735 | ribosomal protein L13 | RPL13 |
| NM_024305 | hypothetical protein MGC4278 | MGC4278 |
| NM_005731 | “actin related protein 2/3 complex, subunit 2, | ARPC2 |
| | 34 kDa” | |
| NM_000982 | ribosomal protein L21 | RPL21 |
| BC006325 | G-2 and S-phase expressed 1 | GTSE1 |
| AA927664 | “transcription elongation factor B (SIII), | TCEB1L |
| | polypeptide 1-like” | |
| AK026525 | glyceraldehyde-3-phosphate dehydrogenase | GAPD |
| NM_014128 | “ferritin, light polypeptide” | FTL |
| NM_017624 | hypothetical protein FLJ20019 | FLJ20019 |
| NM_002870 | “RAB13, member RAS oncogene family” | RAB13 |
| AL535380 | “B-cell translocation gene 1, anti- | BTG1 |
| | proliferative” | |
| AF022991 | period homolog 1 (Drosophila) | PER1 |
| U36764 | “eukaryotic translation initiation factor 3, | EIF3S2 |
| | subunit 2 beta, 36 kDa” | |
| NM_018946 | N-acetylneuraminic acid phosphate synthase; | SAS |
| | sialic acid synthase | |
| AI889513 | Homo sapiens mRNA; cDNA | |
| | DKFZp686D1577 (from clone | |
| | DKFZp686D1577) | |
| AI201594 | Homo sapiens mRNA; cDNA | |
| | DKFZp762M127 (from clone | |
| | DKFZp762M127) | |
| AF064824 | receptor-interacting serine-threonine kinase 2 | RIPK2 |
| BG537190 | “ESTs, Highly similar to FRIL_HUMAN | |
| | Ferritin light chain (Ferritin L subunit) | |
| | [H. sapiens]” | |
| AV741657 | “Homo sapiens cDNA: FLJ21692 fis, clone | |
| | COL09588” | |
| AK000773 | “Homo sapiens cDNA FLJ20766 fis, clone | |
| | COL07978” | |
| AA872727 | farnesyl-diphosphate farnesyltransferase 1 | FDFT1 |
| AW276522 | “solute carrier family 6 (neurotransmitter | SLC6A8 |
| | transporter, creatine), member 8” | |
| NM_025208 | spinal cord-derived growth factor-B | SCDGF-B |
| NM_001530 | “hypoxia-inducible factor 1, alpha subunit | HIF1A |
| | (basic helix-loop-helix transcription factor)” | |
| AI275690 | myeloid cell leukemia sequence 1 (BCL2- | MCL1 |
| | related) | |
| NM_018370 | hypothetical protein FLJ11259 | FLJ11259 |
| BG538564 | “ferritin, light polypeptide” | FTL |
| NM_030978 | “hypothetical protein similar to actin related | MGC3038 |
| | protein 2/3 complex, subunit 5” | |
| AW003091 | “Homo sapiens cDNA FLJ38849 fis, clone | |
| | MESAN2008936” | |
| NM_016516 | tumor antigen SLP-8p | HCC8 |
| AA191576 | “nucleophosmin (nucleolar phosphoprotein | NPM1 |
| | B23, numatrin)” | |
| U27336 | “fucosyltransferase 6 (alpha (1,3) | FUT6 |
| | fucosyltransferase)” | |
| AI378044 | UDP-glucose ceramide glucosyltransferase | UGCG |
| NM_001273 | chromodomain helicase DNA binding protein 4 | CHD4 |
| AA634272 | signal transducer and activator of | STAT3 |
| | transcription 3 (acute-phase response factor) | |
| NM_015367 | MIL1 protein | MIL1 |
| AA443762 | “guanine nucleotide binding protein (G | GNB2L1 |
| | protein), beta polypeptide 2-like 1” | |
| S81916 | “Phosphoglycerate kinase {alternatively | |
| | spliced} [human, phosphoglycerate kinase | |
| | deficient patient with episodes of muscl, | |
| | mRNA Partial Mutant, 307 nt]” | |
| AF125393 | “RAB27A, member RAS oncogene family” | RAB27A |
| NM_001455 | forkhead box O3A | FOXO3A |
| NM_004604 | syntaxin 4A (placental) | STX4A |
| AI819238 | “inhibitor of DNA binding 2, dominant | ID2 |
| | negative helix-loop-helix protein” | |
| NM_014349 | “apolipoprotein L, 3” | APOL3 |
| NM_001616 | “activin A receptor, type II” | ACVR2 |
| AU144792 | “Homo sapiens cDNA FLJ10127 fis, clone | |
| | HEMBA1002973, moderately similar to | |
| | CAMP-DEPENDENT 3′,5′-CYCLIC | |
| | PHOSPHODIESTERASE 4B (EC 3.1.4.17)” | |
| AI537887 | erythrocyte membrane protein band 7.2 | EPB72 |
| | (stomatin) | |
| L76666 | “killer cell immunoglobulin-like receptor, | KIR3DL2 |
| | three domains, long cytoplasmic tail, 2” | |
| NM_001759 | cyclin D2 | CCND2 |
| L32185 | “solute carrier family 11 (proton-coupled | SLC11A1 |
| | divalent metal ion transporters), member 1” | |
| BC005043 | hypothetical protein MGC31957 | MGC31957 |
| AW173157 | Homo sapiens mRNA; cDNA | |
| | DKFZp586G1922 (from clone | |
| | DKFZp586G1922) | |
| N21202 | “Homo sapiens cDNA FLJ35517 fis, clone | |
| | SPLEN2000698” | |
| AB030824 | Kruppel-like factor 5 (intestinal) | KLF5 |
| AL564683 | “CCAAT/enhancer binding protein (C/EBP), | CEBPB |
| | beta” | |
| H93077 | chromosome 5 open reading frame 4 | C5orf4 |
| BC001120 | “lectin, galactoside-binding, soluble, 3 | LGALS3 |
| | (galectin 3)” | |
| M34428 | “Pvt1 oncogene homolog, MYC activator | PVT1 |
| | (mouse)” | |
| NM_012323 | v-maf musculoaponeurotic fibrosarcoma | MAFF |
| | oncogene homolog F (avian) | |
| NM_014918 | carbohydrate (chondroitin) synthase 1 | CHSY1 |
| AU150691 | “Homo sapiens cDNA FLJ10577 fis, clone | |
| | NT2RP2003367” | |
| NM_005565 | lymphocyte cytosolic protein 2 (SH2 domain | LCP2 |
| | containing leukocyte protein of 76 kDa) | |
| NM_004720 | “endothelial differentiation, lysophosphatidic | EDG4 |
| | acid G-protein-coupled receptor, 4” | |
| N25732 | forkhead box O3A | FOXO3A |
| N26005 | “protein phosphatase 1, regulatory (inhibitor) | PPP1R3C |
| | subunit 3C” | |
| NM_002970 | spermidine/spermine N1-acetyltransferase | SAT |
| AL039469 | exosome component Rrp41 | FLJ20591 |
| AL110298 | “solute carrier family 2 (facilitated glucose | SLC2A3 |
| | transporter), member 3” | |
| U62733 | “carnitine palmitoyltransferase I, muscle” | CPT1B |
| NM_000717 | carbonic anhydrase IV | CA4 |
| AJ002572 | transcriptional unit N143 | N143 |
| AF051782 | diaphanous homolog 1 (Drosophila) | DIAPH1 |
| AW973834 | ESTs | |
| NM_004120 | “guanylate binding protein 2, interferon- | GBP2 |
| | inducible” | |
| D26054 | “fructose-1,6-bisphosphatase 1” | FBP1 |
| BC004948 | “Homo sapiens, clone MGC: 10846 | |
| | IMAGE: 3616550, mRNA, complete cds” | |
| H71805 | myeloid cell leukemia sequence 1 (BCL2- | MCL1 |
| | related) | |
| NM_003033 | “sialyltransferase 4A (beta-galactosidase | SIAT4A |
| | alpha-2,3-sialytransferase)” | |
| NM_014361 | contactin 5 | CNTN5 |
| AI631159 | “solute carrier family 2 (facilitated glucose | SLC2A3 |
| | transporter), member 3” | |
| NM_017617 | hypothetical protein FLJ20005 | FLJ20005 |
| BC003070 | GATA binding protein 3 | GATA3 |
| AI971258 | seven in absentia homolog 1 (Drosophila) | SIAH1 |
| NM_002908 | v-rel reticuloendotheliosis viral oncogene | REL |
| | homolog (avian) | |
| NM_014005 | protocadherin alpha 9 | PCDHA9 |
| M81104 | CD34 antigen | CD34 |
| NM_030804 | hypothetical protein DKFZp434E2135 | DKFZP434E2135 |
| AL021977 | v-maf musculoaponeurotic fibrosarcoma | MAFF |
| | oncogene homolog F (avian) | |
| NM_006931 | “solute carrier family 2 (facilitated glucose | SLC2A3 |
| | transporter), member 3” | |
| M81635 | erythrocyte membrane protein band 7.2 | EPB72 |
| | (stomatin) | |
| BF516433 | cylindromatosis (turban tumor syndrome) | CYLD |
| AK027071 | transforming growth factor beta-stimulated | TSC22 |
| | protein TSC-22 | |
| BE677761 | hypothetical protein PRO1584 | PRO1584 |
| L07555 | “CD69 antigen (p60, early T-cell activation | CD69 |
| | antigen)” | |
| AA778684 | “solute carrier family 2 (facilitated glucose | SLC2A3 |
| | transporter), member 3” | |
| NM_024875 | hypothetical protein FLJ12921 | FLJ12921 |
| NM_006502 | “polymerase (DNA directed), eta” | POLH |
| NM_002600 | “phosphodiesterase 4B, cAMP-specific | PDE4B |
| | (phosphodiesterase E4 dunce homolog, | |
| | Drosophila)” | |
| NM_002856 | poliovirus receptor-related 2 (herpesvirus | PVRL2 |
| | entry mediator B) | |
| BC005254 | “C-type (calcium dependent, carbohydrate- | CLECSF2 |
| | recognition domain) lectin, superfamily | |
| | member 2 (activation-induced)” | |
| NM_005346 | heat shock 70 kDa protein 1B | HSPA1B |
| BC000145 | “H1 histone family, member 0” | H1F0 |
| NM_004938 | death-associated protein kinase 1 | DAPK1 |
| L20966 | “phosphodiesterase 4B, cAMP-specific | PDE4B |
| | (phosphodiesterase E4 dunce homolog, | |
| | Drosophila)” | |
| D86586 | stem cell growth factor; lymphocyte secreted | SCGF |
| | C-type lectin | |
| NM_014745 | KIAA0233 gene product | KIAA0233 |
| NM_015136 | stabilin 1 | STAB1 |
| NM_005896 | “isocitrate dehydrogenase 1 (NADP+), | IDH1 |
| | soluble” | |
| AW149405 | neurexin 1 | NRXN1 |
| X72501 | T cell receptor delta locus | TRD |
| NM_024530 | hypothetical protein FLJ23306 | FLJ23306 |
| BC005127 | adipose differentiation-related protein | ADFP |
| AF031824 | cystatin F (leukocystatin) | CST7 |
| NM_002379 | “matrilin 1, cartilage matrix protein” | MATN1 |
| NM_004049 | BCL2-related protein A1 | BCL2A1 |
| NM_000713 | biliverdin reductase B (flavin reductase | BLVRB |
| | (NADPH)) | |
| NM_005767 | purinergic receptor (family A group 5) | P2Y5 |
| AL512728 | Homo sapiens mRNA; cDNA | |
| | DKFZp547P082 (from clone | |
| | DKFZp547P082) | |
| NM_000519 | “hemoglobin, delta” | HBD |
| NM_002727 | “proteoglycan 1, secretory granule” | PRG1 |
| X77737 | “solute carrier family 4, anion exchanger, | SLC4A1 |
| | member 1 (erythrocyte membrane protein | |
| | band 3, Diego blood group)” | |
| AB035266 | neurexin 2 | NRXN2 |
| AV720514 | “ESTs, Weakly similar to hypothetical | |
| | protein FLJ20489 [Homo sapiens] | |
| | [H. sapiens]” | |
| AL562152 | SH3-domain binding protein 5 (BTK- | SH3BP5 |
| | associated) | |
| NM_024703 | hypothetical protein FLJ22593 | FLJ22593 |
| J03223 | “proteoglycan 1, secretory granule” | PRG1 |
| AF153330 | “solute carrier family 19 (thiamine | SLC19A2 |
| | transporter), member 2” | |
| NM_000591 | CD14 antigen | CD14 |
| BG035761 | suppressor of cytokine signaling 3 | SSI-3 |
| AB014511 | “ATPase, Class II, type 9A” | ATP9A |
| AF130113 | | |

TABLE 7A
Top genes discriminating vincristine resistant and sensitive B-
lineage ALL: Genes down-regulated in vincristine resistant
B-lineage ALL

| | | | | NCBI |
|---|---|---|---|---|
| | | Gene | R/S | Accession |
| Probe ID | Gene Name | Symbol | ratio | Number |
| 200593_s_at | heterogeneous nuclear | HNRPU | 0.76 | BC003621 |
| | ribonucleoprotein U | | | |
| 202209_at | LSM3 homolog, U6 | LSM3 | 0.77 | NM_014463 |
| | small nuclear RNA | | | |
| | associated | | | |
| 217733_s_at | thymosin, beta 10 | TMSB10 | 0.78 | NM_021103 |
| 200031_s_at | ribosomal protein S11 | RPS11 | 0.84 | NM_001015 |
| 200088_x_at | cDNA (AK026491) | | 0.79 | AK026491 |
| 213377_x_at | complement | C1S | 0.78 | AI799007 |
| | component 1, s | | | |
| | subcomponent | | | |
| 200781_s_at | ribosomal protein | RPS15A | 0.81 | NM_001019 |
| | S15a | | | |
| 200909_s_at | ribosomal protein, | RPLP2 | 0.85 | NM_001004 |
| | large P2 | | | |
| 218672_at | hypothetical protein | MGC3180 | 0.70 | NM_024041 |
| | MGC3180 | | | |
| 208690_s_at | PDZ and LIM domain | PDLIM1 | 0.53 | BC000915 |
| | 1 (elfin) | | | |
| 218608_at | putative ATPase | HSA9947 | 0.10 | NM_022089 |
| 202598_at | S100 calcium binding | S100A13 | 0.69 | NM_005979 |
| | protein A13 | | | |
| 201268_at | non-metastatic cells 2, | NME2 | 0.67 | NM_002512 |
| | protein (NM23B) | | | |
| | expressed in | | | |
| 210240_s_at | cyclin-dependent | CDKN2D | | U20498 |
| | kinase inhibitor 2D | 0.66 | | |
| | (p19, inhibits CDK4) | | | |
| 201426_s_at | ribosomal protein, | RPLP2 | 0.52 | AI922599 |
| | large P2 | | | |
| 217523_at | CD44 antigen | CD44 | 0.30 | AV700298 |
| 204490_s_at | CD44 antigen | CD44 | 0.36 | M24915 |

TABLE 7B
Top genes discriminating vincristine resistant and sensitive B-
lineage ALL: Genes up-regulated in vincristine resistant B-lineage ALL

| | | | | NCBI |
|---|---|---|---|---|
| | | | R/S | Accession |
| Probe ID | Gene Name | Gene Symbol | ratio | Number |
| 218325_s_at | death associated | DATF1 | 1.59 | NM_022105 |
| | transcription factor 1 | | | |
| 207753_at | zinc finger protein 304 | ZNF304 | 1.66 | NM_020657 |
| 201851_at | SH3-domain GRB2- | SH3GL1 | 1.52 | NM_003025 |
| | like 1 | | | |
| 210162_s_at | nuclear factor of | NFATC1 | 1.80 | U08015 |
| | activated T-cells, | | | |
| | cytoplasmic, | | | |
| | calcineurin-depe | | | |
| 213831_at | major | HLA-DQA1 | 3.55 | X00452 |
| | histocompatibility | | | |
| | complex, class II, DQ alpha 1 | | | |
| 215127_s_at | RNA binding motif, | RBMS1 | 1.38 | AL517946 |
| | single stranded | | | |
| | interacting protein 1 | | | |
| 218647_s_at | hypothetical protein | FLJ23476 | 1.28 | NM_024640 |
| | FLJ23476 | | | |
| 204914_s_at | ESTs, Weakly similar | | 17.68 | AI360875 |
| | to F36H12.3.p | | | |
| 219528_s_at | B-cell CLL/lymphoma | BCL11B | 2.11 | NM_022898 |
| | 11B (zinc finger | | | |
| | protein) | | | |
| 213604_at | Homo sapiens clone | | 1.25 | AW451236 |
| | 24582 mRNA | | | |
| | sequence | | | |
| 212037_at | pinin, desmosome | PNN | 1.49 | Y09703 |
| | associated protein | | | |
| 202392_s_at | phosphatidylserine | PISD | 1.43 | NM_014338 |
| | decarboxylase | | | |
| 212492_s_at | KIAA0876 protein | KIAA0876 | 1.46 | AW237172 |
| 209705_at | ESTs | | 1.30 | AF073293 |
| 204837_at | myotubularin related | MTMR9 | 1.40 | AL080178 |
| | protein 9 | | | |
| 212438_at | putative nucleic acid | RY1 | 1.57 | BG252325 |
| | binding protein RY-1 | | | |
| 200757_s_at | calumenin | CALU | 1.28 | NM_001219 |
| 202946_s_at | BTB (POZ) domain | BTBD3 | 1.79 | NM_014962 |
| | containing 3 | | | |
| 55872_at | KIAA1196 protein | KIAA1196 | 1.54 | AI493119 |
| 218951_s_at | hypothetical protein | FLJ11323 | 1.69 | NM_018390 |
| | FLJ11323 | | | |
| 213939_s_at | Homo sapiens cDNA | | 4.12 | AI871641 |
| | FLJ12012 fis, clone | | | |
| | HEMBB1001668. | | | |
| 212222_at | proteasome activator | PA200 | 1.40 | D38521 |
| | 200 kDa | | | |
| 206033_s_at | desmocollin 3 | DSC3 | 1.72 | NM_001941 |
| 202668_at | ephrin-B2 | EFNB2 | 2.15 | BF001670 |
| 218642_s_at | hypothetical protein | MGC2217 | 1.34 | NM_024300 |
| | MGC2217 | | | |
| 212345_s_at | hypothetical protein | DKFZP586F2423 | 1.78 | BE675139 |
| | DKFZp586F2423 | | | |
| 203910_at | PTPL1-associated | PARG1 | 2.42 | NM_004815 |
| | RhoGAP 1 | | | |
| 213301_x_at | transcriptional | TIF1 | 2.55 | AL538264 |
| | intermediary factor 1 | | | |
| 204391_x_at | transcriptional | TIF1 | 2.91 | NM_015905 |
| | intermediary factor 1 | | | |
| 204849_at | transcription factor- | TCFL5 | 5.26 | NM_006602 |
| | like 5 (basic helix- | | | |
| | loop-helix) | | | |
| 221011_s_at | likely ortholog of | LBH | 1.36 | NM_030915 |
| | mouse limb-bud and | | | |
| | heart gene | | | |
| 213017_at | abhydrolase domain | ABHD3 | 2.69 | AL534702 |
| | containing 3 | | | |
| 206231_at | potassium | KCNN1 | 3.29 | NM_002248 |
| | intermediate/small | | | |
| | conductance calcium- | | | |
| | activated ch | | | |
| 202517_at | collapsin response | CRMP1 | 2.59 | NM_001313 |
| | mediator protein 1 | | | |
| 209296_at | protein phosphatase | PPM1B | 1.40 | AF136972 |
| | 1B (formerly 2C), | | | |
| | magnesium-dependent | | | |
| 212956_at | KIAA0882 protein | KIAA0882 | 3.64 | AB020689 |
| 219471_at | chromosome 13 open | C13orf18 | 2.31 | NM_025113 |
| | reading frame 18 | | | |
| 44790_s_at | chromosome 13 open | C13orf18 | 3.13 | AI129310 |
| | reading frame 18 | | | |
| 204434_at | spermatogenesis | SPATA2 | 1.36 | NM_006038 |
| | associated 2 | | | |
| 203707_at | zinc finger protein 263 | ZNF263 | 1.32 | NM_005741 |
| 213225_at | protein phosphatase | PPM1B | 1.45 | AJ271832 |
| | 1B (formerly 2C), | | | |
| | magnesium-dependent | | | |
| 213360_s_at | POM121 membrane | POM121 | 1.32 | AA514622 |
| | glycoprotein (rat) | | | |

TABLE 7C
Genes discriminating vincristine resistant and sensitive B-lineage
ALL to the 0.001 < p < 0.01 level by both T-test and Wilcoxon: Genes down-
regulated in vincristine resistant B-lineage ALL

| NCBI | | |
|---|---|---|
| Accession | | |
| Number | Gene Name | Gene Symbol |
| NM_022089 | putative ATPase | HSA9947 |
| NM_018465 | uncharacterized hematopoietic stem/progenitor | MDS030 |
| | cells protein MDS030 | |
| NM_016071 | mitochondrial ribosomal protein S33 | MRPS33 |
| AV700298 | “ESTs, Highly similar to CD44_HUMAN CD44 | |
| | antigen precursor (Phagocytic glycoprotein I) | |
| | (PGP-1) (HUTCH-I) (Extracellular matrix | |
| | receptor-III) (ECMR-III) (GP90 lymphocyte | |
| | homing/adhesion receptor) (Hermes antigen) | |
| | (Hyaluronate receptor) (Heparan sulfate pro | |
| AB028839 | “ubiquitination factor E4B (UFD2 homolog, | UBE4B |
| | yeast)” | |
| NM_002452 | nudix (nucleoside diphosphate linked moiety X)-type | NUDT1 |
| | motif 1 | |
| BF513430 | DKFZP586J0619 protein | DKFZP586J0619 |
| BC002906 | uridine monophosphate kinase | UMPK |
| Z00008 | | |
| NM_003645 | “solute carrier family 27 (fatty acid transporter), | SLC27A2 |
| | member 2” | |
| BG340670 | immunoglobulin heavy constant mu | IGHM |
| M24915 | CD44 antigen (homing function and Indian blood | CD44 |
| | group system) | |
| AV764378 | “RNA, U2 small nuclear” | RNU2 |
| BC004372 | CD44 antigen (homing function and Indian blood | CD44 |
| | group system) | |
| AI493245 | CD44 antigen (homing function and Indian blood | CD44 |
| | group system) | |
| NM_002339 | lymphocyte-specific protein 1 | LSP1 |
| NM_001553 | insulin-like growth factor binding protein 7 | IGFBP7 |
| NM_017518 | three prime repair exonuclease 2 | TREX2 |
| NM_002961 | “S100 calcium binding protein A4 (calcium | S100A4 |
| | protein, calvasculin, metastasin, murine placental | |
| | homolog)” | |
| NM_002765 | phosphoribosyl pyrophosphate synthetase 2 | PRPS2 |
| AF098641 | “Homo sapiens CD44 isoform RC (CD44) | |
| | mRNA, complete cds” | |
| NM_004385 | chondroitin sulfate proteoglycan 2 (versican) | CSPG2 |
| NM_004039 | annexin A2 | ANXA2 |
| NM_003537 | “H3 histone family, member L” | H3FL |
| NM_000610 | CD44 antigen (homing function and Indian blood | CD44 |
| | group system) | |
| BC003068 | “solute carrier family 19 (folate transporter), | SLC19A1 |
| | member 1” | |
| AF199015 | villin 2 (ezrin) | VIL2 |
| BE903880 | CD44 antigen (homing function and Indian blood | CD44 |
| | group system) | |
| NM_004284 | chromodomain helicase DNA binding protein 1- | CHD1L |
| | like | |
| BE908217 | annexin A2 | ANXA2 |
| AA476303 | immunoglobulin kappa constant | IGKC |
| L38969 | thrombospondin 3 | THBS3 |
| NM_001819 | chromogranin B (secretogranin 1) | CHGB |
| NM_006907 | pyrroline-5-carboxylate reductase 1 | PYCR1 |
| BC001388 | annexin A2 | ANXA2 |
| NM_002712 | “protein phosphatase 1, regulatory subunit 7” | PPP1R7 |
| NM_021827 | hypothetical protein FLJ23514 | FLJ23514 |
| BC000915 | PDZ and LIM domain 1 (elfin) | PDLIM1 |
| BC000988 | hypothetical protein MGC5457 | MGC5457 |
| NM_000626 | CD79B antigen (immunoglobulin-associated beta) | CD79B |
| M28882 | melanoma cell adhesion molecule | MCAM |
| NM_000884 | IMP (inosine monophosphate) dehydrogenase 2 | IMPDH2 |
| AI832239 | mitochondrial ribosomal protein L23 | MRPL23 |
| AL525086 | UDP-N-acetyl-alpha-D- | GALNT2 |
| | galactosamine:polypeptide N- | |
| | acetylgalactosaminyltransferase 2 (GalNAc-T2) | |
| AI922599 | vimentin | VIM |
| AA112507 | U6 snRNA-associated Sm-like protein | LSM4 |
| AI688812 | RAS guanyl releasing protein 2 (calcium and | RASGRP2 |
| | DAG-regulated) | |
| U13699 | “caspase 1, apoptosis-related cysteine protease | CASP1 |
| | (interleukin 1, beta, convertase)” | |
| BC004291 | hypothetical protein MGC17226 | MGC17226 |
| L04636 | “complement component 1, q subcomponent | C1QBP |
| | binding protein” | |
| NM_001428 | “enolase 1, (alpha)” | ENO1 |
| AV748469 | SEC14-like 1 (S. cerevisiae) | SEC14L1 |
| NM_014257 | CD209 antigen-like | CD209L |
| NM_030796 | hypothetical protein DKFZp564K0822 | DKFZP564K0822 |
| AV655640 | “CCAAT/enhancer binding protein (C/EBP), | CEBPD |
| | delta” | |
| NM_004089 | “delta sleep inducing peptide, immunoreactor” | DSIPI |
| AF089868 | melanoma cell adhesion molecule | MCAM |
| NM_003931 | “WAS protein family, member 1” | WASF1 |
| U20498 | “cyclin-dependent kinase inhibitor 2D (p19, | CDKN2D |
| | inhibits CDK4)” | |
| NM_003916 | “adaptor-related protein complex 1, sigma 2 | AP1S2 |
| | subunit” | |
| NM_005979 | S100 calcium binding protein A13 | S100A13 |
| NM_006817 | chromosome 12 open reading frame 8 | C12orf8 |
| NM_024041 | hypothetical protein MGC3180 | MGC3180 |
| AF061735 | “ATP synthase, H+ transporting, mitochondrial | ATP5H |
| | F0 complex, subunit d” | |
| BE891920 | “actin related protein 2/3 complex, subunit 4, | ARPC4 |
| | 20 kDa” | |
| J04423 | | |
| BG395660 | “ubiquitin-conjugating enzyme E2G 2 (UBC7 | UBE2G2 |
| | homolog, yeast)” | |
| NM_012458 | translocase of inner mitochondrial membrane 13 | TIMM13 |
| | homolog (yeast) | |
| NM_004078 | cysteine and glycine-rich protein 1 | CSRP1 |
| NM_002512 | “non-metastatic cells 2, protein (NM23B) | NME2 |
| | expressed in” | |
| NM_004271 | “MD-1, RP105-associated” | MD-1 |
| NM_005174 | “ATP synthase, H+ transporting, mitochondrial | ATP5C1 |
| | F1 complex, gamma polypeptide 1” | |
| AI669379 | “Homo sapiens cDNA FLJ35429 fis, clone | |
| | SMINT2002126” | |
| NM_006294 | ubiquinol-cytochrome c reductase binding protein | UQCRB |
| J04423 | | |
| J04423 | | |
| NM_001697 | “ATP synthase, H+ transporting, mitochondrial | ATP5O |
| | F1 complex, O subunit (oligomycin sensitivity | |
| | conferring protein)” | |
| NM_004374 | cytochrome c oxidase subunit VIc | COX6C |
| AA406605 | weakly similar to glutathione peroxidase 2 | CL683 |
| AF199015 | villin 2 (ezrin) | VIL2 |
| NM_014463 | Lsm3 protein | LSM3 |
| NM_003134 | signal recognition particle 14 kDa (homologous | SRP14 |
| | Alu RNA binding protein) | |
| J04423 | | |
| M26700 | ubiquinol-cytochrome c reductase binding protein | UQCRB |
| BE675435 | core promoter element binding protein | COPEB |
| NM_002128 | high-mobility group box 1 | HMGB1 |
| BC000931 | “ATP synthase, H+ transporting, mitochondrial | ATP5C1 |
| | F1 complex, gamma polypeptide 1” | |
| AV702405 | emopamil binding protein (sterol isomerase) | EBP |
| BC003621 | heterogeneous nuclear ribonucleoprotein U | HNRPU |
| | (scaffold attachment factor A) | |
| AW451954 | adaptor-associated kinase 1 | AAK1 |
| U13698 | “caspase 1, apoptosis-related cysteine protease | CASP1 |
| | (interleukin 1, beta, convertase)” | |
| AB035745 | Down syndrome critical region gene 5 | DSCR5 |
| M29277 | melanoma cell adhesion molecule | MCAM |
| NM_003611 | oral-facial-digital syndrome 1 | OFD1 |
| AF116273 | BCL2-associated athanogene | BAG1 |
| BF669264 | “Human erbB3 binding protein EBP1 mRNA, | |
| | complete cds” | |
| BE311760 | high-mobility group box 1 | HMGB1 |
| NM_016199 | U6 snRNA-associated Sm-like protein LSm7 | LOC51690 |
| AF116639 | chromosome 14 open reading frame 2 | C14orf2 |
| NM_003756 | “eukaryotic translation initiation factor 3, subunit | EIF3S3 |
| | 3 gamma, 40 kDa” | |
| AA555113 | “ribosomal protein, large, P0” | RPLP0 |
| AL136179 | SRY (sex determining region Y)-box 4 | SOX4 |
| AU151801 | “complement component 1, q subcomponent | C1QBP |
| | binding protein” | |
| AL110191 | “delta sleep inducing peptide, immunoreactor” | DSIPI |
| NM_031286 | SH3 domain binding glutamic acid-rich protein | SH3BGRL3 |
| | like 3 | |
| BC001865 | “ESTs, Highly similar to ribosomal protein S2; | |
| | 40S ribosomal protein S2 [Homo sapiens] | |
| | [H. sapiens]” | |
| AF348514 | “prothymosin, alpha (gene sequence 28)” | PTMA |
| X03453 | | |
| NM_021103 | “thymosin, beta 10” | TMSB10 |
| BF125158 | ribosomal protein S2 | RPS2 |
| AF257099 | | |
| AK026491 | ribosomal protein L12 | RPL12 |
| BC006483 | ribosomal protein L3 | RPL3 |
| AL133228 | | |
| AI799007 | ribosomal protein S12 | RPS12 |
| NM_000967 | ribosomal protein L3 | RPL3 |
| J04423 | | |
| BC001360 | “ras homolog gene family, member A” | ARHA |
| NM_000984 | ribosomal protein L23a | RPL23A |
| BC000523 | ribosomal protein S24 | RPS24 |
| L22453 | | |
| U43701 | ribosomal protein L23a | RPL23A |
| BC000514 | ribosomal protein L13a | RPL13A |
| BC006337 | “adaptor-related protein complex 2, sigma 1 | AP2S1 |
| | subunit” | |
| AA838274 | ribosomal protein L14 | RPL14 |
| AA630314 | ribosomal protein S2 | RPS2 |
| J04423 | | |
| NM_001025 | ribosomal protein S23 | RPS23 |
| BE305165 | “protein phosphatase 1, regulatory (inhibitor) | PPP1R14B |
| | subunit 14B” | |
| NM_000973 | ribosomal protein L8 | RPL8 |
| NM_001207 | basic transcription factor 3 | BTF3 |
| NM_001003 | “ribosomal protein, large, P1” | RPLP1 |
| NM_001403 | cyclin D1 (PRAD1: parathyroid adenomatosis 1) | CCND1 |
| NM_000980 | ribosomal protein L18a | RPL18A |
| NM_001019 | ribosomal protein S15a | RPS15A |
| AA281332 | ribosomal protein L12 | RPL12 |
| NM_001001 | ribosomal protein L36a-like | RPL36AL |
| AW188940 | beta-2-microglobulin | B2M |
| AB009010 | ubiquitin C | UBC |
| NM_000976 | ribosomal protein L12 | RPL12 |
| X74070 | basic transcription factor 3 | BTF3 |
| NM_022551 | ribosomal protein S18 | RPS18 |
| NM_001402 | eukaryotic translation elongation factor 1 alpha 1 | EEF1A1 |
| NM_001007 | “ribosomal protein S4, X-linked” | RPS4X |
| NM_000971 | ribosomal protein L7 | RPL7 |
| NM_006013 | ribosomal protein L10 | RPL10 |
| NM_000993 | ribosomal protein L31 | RPL31 |
| NM_001015 | ribosomal protein S11 | RPS11 |
| X03453 | | |
| NM_012423 | ribosomal protein L13a | RPL13A |
| NM_000990 | ribosomal protein L27a | RPL27A |
| NM_001032 | ribosomal protein S29 | RPS29 |
| L05095 | hypothetical protein FLJ22875 | FLJ22875 |
| BC003655 | “ribosomal protein, large, P0” | RPLP0 |
| NM_001017 | ribosomal protein S13 | RPS13 |
| NM_000981 | ribosomal protein L19 | RPL19 |
| NM_021029 | ribosomal protein L36A | RPL36A |
| AI953822 | “ribosomal protein, large, P0” | RPLP0 |
| AI734929 | “poly(A) binding protein, cytoplasmic 1” | PABPC1 |
| NM_001004 | “ribosomal protein, large P2” | RPLP2 |
| AI721229 | hypothetical protein FLJ20030 | FLJ20030 |
| BE786672 | eukaryotic translation elongation factor 1 alpha 1 | EEF1A1 |
| BC001675 | ribosomal protein L13a | RPL13A |
| J04423 | | |
| AI183766 | ribosomal protein S2 | RPS2 |
| NM_001002 | “ribosomal protein, large, P0” | RPLP0 |
| BF686442 | “prothymosin, alpha (gene sequence 28)” | PTMA |
| BC001019 | ribosomal protein L39 | RPL39 |
| AF116710 | ribosomal protein S14 | RPS14 |
| BC000354 | ribosomal protein S28 | RPS28 |
| AK026525 | glyceraldehyde-3-phosphate dehydrogenase | GAPD |
| BF942308 | ribosomal protein L13a | RPL13A |
| AF072098 | | |
| NM_021104 | ribosomal protein L41 | RPL41 |
| BE964125 | eukaryotic translation elongation factor 1 alpha 1 | EEF1A1 |

TABLE 7D
Genes discriminating vincristine resistant and sensitive B-lineage
ALL to the 0.001 < p < 0.01 level by both T-test and Wilcoxon:
Genes up-regulated in vincristine resistant B-lineage ALL

| NCBI | | |
|---|---|---|
| Accession Number | Gene Name | Gene Symbol |
| NM_024116 | hypothetical protein MGC5306 | MGC5306 |
| BF690062 | polypyrimidine tract binding protein 1 | PTBP1 |
| X62006 | polypyrimidine tract binding protein 1 | PTBP1 |
| NM_005626 | “splicing factor, arginine/serine-rich 4” | SFRS4 |
| NM_003663 | CGG triplet repeat binding protein 1 | CGGBP1 |
| NM_025160 | hypothetical protein FLJ21016 | FLJ21016 |
| L35253 | mitogen-activated protein kinase 14 | MAPK14 |
| AF072814 | likely ortholog of mouse metal response element | M96 |
| | binding transcription factor 2 | |
| NM_007375 | TAR DNA binding protein | TARDBP |
| NM_005109 | oxidative-stress responsive 1 | OSR1 |
| NM_016146 | PTD009 protein | PTD009 |
| NM_005759 | abl-interactor 2 | ABI-2 |
| AL041728 | slingshot 1 | SSH1 |
| AI344075 | gamma-glutamyltransferase 2 | GGT2 |
| AI769637 | UDP-glucuronic acid/UDP-N- | UGTREL7 |
| | acetylgalactosamine dual transporter | |
| NM_017706 | hypothetical protein FLJ20195 | FLJ20195 |
| NM_000801 | “FK506 binding protein 1A, 12 kDa” | FKBP1A |
| NM_002537 | ornithine decarboxylase antizyme 2 | OAZ2 |
| NM_018282 | paraspeckle protein 1 | PSP1 |
| AK024029 | modulator of apoptosis 1 | MAP-1 |
| NM_023924 | hypothetical protein FLJ13441 | FLJ13441 |
| NM_018146 | putative RNA methyltransferase | FLJ10581 |
| AI133727 | likely ortholog of rat zinc-finger antiviral protein | ZAP |
| AF104913 | “eukaryotic translation initiation factor 4 gamma, 1” | EIF4G1 |
| AK023188 | “protein phosphatase 1, regulatory (inhibitor) | PPP1R13B |
| | subunit 13B” | |
| NM_012124 | “cysteine and histidine-rich domain (CHORD)- | CHORDC1 |
| | containing, zinc binding protein 1” | |
| NM_014517 | upstream binding protein 1 (LBP-1a) | UBP1 |
| AL514076 | sarcoma amplified sequence | SAS |
| NM_030955 | “a disintegrin-like and metalloprotease | ADAMTS12 |
| | (reprolysin type) with thrombospondin type 1 | |
| | motif, 12” | |
| AV682285 | RE1-silencing transcription factor | REST |
| NM_005428 | vav 1 oncogene | VAV1 |
| NM_002130 | 3-hydroxy-3-methylglutaryl-Coenzyme A | HMGCS1 |
| | synthase 1 (soluble) | |
| NM_017895 | DEAD/H (Asp-Glu-Ala-Asp/His) box | DDX27 |
| | polypeptide 27 | |
| NM_004738 | VAMP (vesicle-associated membrane protein)- | VAPB |
| | associated protein B and C | |
| AW299555 | “ubiquitin-conjugating enzyme E2G 1 (UBC7 | UBE2G1 |
| | homolog, C. elegans)” | |
| AA156948 | PRP4 pre-mRNA processing factor 4 homolog B | PRPF4B |
| | (yeast) | |
| NM_017892 | formin binding protein 3 | FNBP3 |
| NM_002266 | “karyopherin alpha 2 (RAG cohort 1, importin | KPNA2 |
| | alpha 1)” | |
| AL096828 | chromosome 20 open reading frame 21 | C20orf21 |
| AA514622 | KIAA0618 gene product | KIAA0618 |
| AL120741 | apyrase | SHAPY |
| NM_018032 | LUC7-like (S. cerevisiae) | LUC7L |
| AI638771 | “phosphate cytidylyltransferase 1, choline, alpha | PCYT1A |
| | isoform” | |
| U17714 | suppression of tumorigenicity 13 (colon | ST13 |
| | carcinoma) (Hsp70 interacting protein) | |
| AF254083 | zinc finger protein 278 | ZNF278 |
| AW664421 | suppressor of cytokine signaling 5 | SOCS5 |
| AW451236 | Homo sapiens clone 24582 mRNA sequence | |
| NM_015626 | SOCS box-containing WD protein SWiP-1 | WSB1 |
| NM_024640 | hypothetical protein FLJ23476 | FLJ23476 |
| BC002557 | hypothetical protein MGC2306 | MGC2306 |
| NM_002131 | high mobility group AT-hook 1 | HMGA1 |
| NM_005044 | “protein kinase, X-linked” | PRKX |
| NM_014247 | PDZ domain containing guanine nucleotide | PDZ-GEF1 |
| | exchange factor(GEF)1 | |
| AL136598 | protein associated with PRK1 | AWP1 |
| AL110238 | DKFZP566E104 protein | DKFZP566E104 |
| BC005247 | isopentenyl-diphosphate delta isomerase | IDI1 |
| NM_014577 | bromodomain containing 1 | BRD1 |
| AI744148 | KIAA0431 protein | KIAA0431 |
| NM_006999 | polymerase (DNA directed) sigma | POLS |
| NM_002657 | pleiomorphic adenoma gene-like 2 | PLAGL2 |
| NM_018182 | hypothetical protein FLJ10700 | FLJ10700 |
| NM_017896 | chromosome 20 open reading frame 11 | C20orf11 |
| NM_012406 | PR domain containing 4 | PRDM4 |
| BG033764 | likely ortholog of mouse metal response element | M96 |
| | binding transcription factor 2 | |
| AI525402 | hypothetical protein FLJ11939 | FLJ11939 |
| AI123320 | “eukaryotic translation initiation factor 3, subunit | EIF3S10 |
| | 10 theta, 150/170 kDa” | |
| BC001686 | “methionine adenosyltransferase II, alpha” | MAT2A |
| AI937543 | DC12 protein | DC12 |
| AL136896 | suppressor of cytokine signaling 5 | SOCS5 |
| NM_001219 | calumenin | CALU |
| AL050318 | TGFB-induced factor 2 (TALE family | TGIF2 |
| | homeobox) | |
| N48361 | Homo sapiens mRNA; cDNA DKFZp564O1016 | |
| | (from clone DKFZp564O1016) | |
| NM_003430 | “zinc finger protein 91 (HPF7, HTF10)” | ZNF91 |
| X76302 | putative nucleic acid binding protein RY-1 | RY1 |
| AL571424 | NS1-associated protein 1 | NSAP1 |
| NM_005741 | zinc finger protein 263 | ZNF263 |
| NM_021031 | | |
| BC005147 | “FK506 binding protein 1A, 12 kDa” | FKBP1A |
| NM_001046 | “solute carrier family 12 | SLC12A2 |
| | (sodium/potassium/chloride transporters), | |
| | member 2” | |
| AW003091 | “Homo sapiens cDNA FLJ38849 fis, clone | |
| | MESAN2008936” | |
| NM_002056 | glutamine-fructose-6-phosphate transaminase 1 | GFPT1 |
| NM_015959 | CGI-31 protein | LOC51075 |
| NM_015973 | galanin-related peptide | LOC51083 |
| BG292233 | insulin induced gene 1 | INSIG1 |
| BC004191 | dynactin 4 | MGC3248 |
| Z97056 | Homo sapiens mRNA; cDNA DKFZp586K2322 | |
| | (from clone DKFZp586K2322) | |
| NM_003330 | thioredoxin reductase 1 | TXNRD1 |
| NM_022118 | cutaneous T-cell lymphoma tumor antigen se70-2 | SE70-2 |
| AF254088 | zinc finger protein 278 | ZNF278 |
| NM_004368 | calponin 2 | CNN2 |
| NM_016598 | “zinc finger, DHHC domain containing 3” | ZDHHC3 |
| D89053 | “fatty-acid-Coenzyme A ligase, long-chain 3” | FACL3 |
| AA156865 | Homo sapiens mRNA; cDNA DKFZp564O0122 | |
| | (from clone DKFZp564O0122) | |
| NM_006526 | zinc finger protein 217 | ZNF217 |
| NM_030825 | | |
| AF073310 | insulin receptor substrate 2 | IRS2 |
| AI141670 | hypothetical protein MGC21688 | MGC21688 |
| AK022555 | KIAA0618 gene product | KIAA0618 |
| NM_024300 | hypothetical protein MGC2217 | MGC2217 |
| NM_013316 | “CCR4-NOT transcription complex, subunit 4” | CNOT4 |
| NM_030915 | likely ortholog of mouse limb-bud and heart gene | LBH |
| BC000927 | poly(A) polymerase alpha | PAPOLA |
| BE963245 | F-box and WD-40 domain protein 1B | FBXW1B |
| AU145005 | Sp3 transcription factor | SP3 |
| NM_015339 | activity-dependent neuroprotector | ADNP |
| BG536224 | immunoglobulin kappa constant | IGKC |
| NM_007358 | likely ortholog of mouse metal response element | M96 |
| | binding transcription factor 2 | |
| NM_020119 | likely ortholog of rat zinc-finger antiviral protein | ZAP |
| AB014593 | synovial sarcoma translocation gene on | SS18L1 |
| | chromosome 18-like 1 | |
| AI806207 | SNF-1 related kinase | SNRK |
| NM_018043 | hypothetical protein FLJ10261 | FLJ10261 |
| AF136972 | “protein phosphatase 1B (formerly 2C), | PPM1B |
| | magnesium-dependent, beta isoform” | |
| BF590997 | hypothetical protein FLJ10707 | FLJ10707 |
| NM_006038 | spermatogenesis associated 2 | SPATA2 |
| AL517946 | “RNA binding motif, single stranded interacting | RBMS1 |
| | protein 1” | |
| NM_002647 | “phosphoinositide-3-kinase, class 3” | PIK3C3 |
| D26069 | “centaurin, beta 2” | CENTB2 |
| NM_016265 | GIOT-3 for gonadotropin inducible transcription | GIOT-3 |
| | repressor-3 | |
| NM_031214 | hypothetical protein AF311304 | AF311304 |
| NM_025078 | hypothetical protein FLJ22378 | FLJ22378 |
| NM_003031 | seven in absentia homolog 1 (Drosophila) | SIAH1 |
| NM_014011 | suppressor of cytokine signaling 5 | SOCS5 |
| NM_014887 | hypothetical protein from BCRA2 region | CG005 |
| AL080178 | myotubularin related protein 9 | MTMR9 |
| D50911 | KIAA0121 gene product | KIAA0121 |
| AW470003 | KIAA0143 protein | KIAA0143 |
| AF009267 | Homo sapiens clone FBA1 Cri-du-chat region | |
| | mRNA | |
| NM_019034 | “ras homolog gene family, member F (in | ARHF |
| | filopodia)” | |
| BE256900 | KIAA0876 protein | KIAA0876 |
| NM_018616 | hypothetical protein PRO2037 | PRO2037 |
| N22859 | KIAA0934 protein | KIAA0934 |
| AB002344 | KIAA0346 protein | KIAA0346 |
| X72631 | “nuclear receptor subfamily 1, group D, member | NR1D1 |
| | 1” | |
| NM_019591 | zinc finger protein 26 (KOX 20) | ZNF26 |
| AU143855 | proteasome activator 200 kDa | PA200 |
| AI333651 | frizzled homolog 7 (Drosophila) | FZD7 |
| AA129753 | “Rab geranylgeranyltransferase, beta subunit” | RABGGTB |
| NM_014338 | phosphatidylserine decarboxylase | PISD |
| AW237172 | KIAA0876 protein | KIAA0876 |
| AJ297710 | cell division cycle 2-like 5 (cholinesterase-related | CDC2L5 |
| | cell division controller) | |
| AJ271832 | “protein phosphatase 1B (formerly 2C), | PPM1B |
| | magnesium-dependent, beta isoform” | |
| BF222893 | protocadherin 16 dachsous-like (Drosophila) | PCDH16 |
| AA830884 | fragile X mental retardation 1 | FMR1 |
| AW592227 | “KH domain containing, RNA binding, signal | KHDRBS1 |
| | transduction associated 1” | |
| BF969986 | hypothetical protein FLJ38045 | FLJ38045 |
| AI823592 | KIAA0423 protein | KIAA0423 |
| AB023227 | KIAA1010 protein | KIAA1010 |
| BF508848 | “pinin, desmosome associated protein” | PNN |
| NM_003025 | SH3-domain GRB2-like 1 | SH3GL1 |
| NM_003928 | CAAX box 1 | CXX1 |
| NM_018200 | high-mobility group 20A | HMG20A |
| AA554945 | “ESTs, Weakly similar to hypothetical protein | |
| | FLJ20378 [Homo sapiens] [H. sapiens]” | |
| AW167793 | glucosamine (N-acetyl)-6-sulfatase (Sanfilippo | GNS |
| | disease IIID) | |
| NM_017792 | hypothetical protein FLJ20373 | FLJ20373 |
| AF052128 | immunoglobulin mu binding protein 2 | IGHMBP2 |
| NM_001241 | cyclin T2 | CCNT2 |
| AK025298 | autism-related protein 1 | KIAA0442 |
| NM_005180 | hypothetical protein MGC12685 | MGC12685 |
| AI493119 | KIAA1196 protein | KIAA1196 |
| AI829875 | B-cell CLL/lymphoma 3 | BCL3 |
| NM_018274 | | |
| AI700633 | “Homo sapiens cDNA: FLJ22642 fis, clone | |
| | HSI06970” | |
| NM_005187 | “core-binding factor, runt domain, alpha subunit | CBFA2T3 |
| | 2; translocated to, 3” | |
| AW293531 | N-myristoyltransferase 2 | NMT2 |
| NM_006235 | “POU domain, class 2, associating factor 1” | POU2AF1 |
| BG252325 | putative nucleic acid binding protein RY-1 | RY1 |
| NM_002162 | intercellular adhesion molecule 3 | ICAM3 |
| BC000277 | KIAA0712 gene product | KIAA0712 |
| BE256900 | KIAA0876 protein | KIAA0876 |
| NM_017610 | likely ortholog of mouse Arkadia | DKFZp761D081 |
| AF186779 | RalGDS-like gene | RGL |
| AF055024 | ankyrin repeat and SOCS box-containing 1 | ASB1 |
| AA485908 | insulin receptor | INSR |
| NM_016839 | “RNA binding motif, single stranded interacting | RBMS1 |
| | protein 1” | |
| NM_012068 | activating transcription factor 5 | ATF5 |
| NM_018114 | hypothetical protein FLJ10496 | FLJ10496 |
| NM_014426 | sorting nexin 5 | SNX5 |
| BE042976 | hypothetical protein MGC17330 | MGC17330 |
| NM_021255 | pellino homolog 2 (Drosophila) | PELI2 |
| NM_018390 | hypothetical protein FLJ11323 | FLJ11323 |
| AK001821 | MGC4170 protein | MGC4170 |
| BF439316 | transmembrane protein with EGF-like and two | TMEFF1 |
| | follistatin-like domains 1 | |
| AF070647 | Homo sapiens clone 24438 mRNA sequence | |
| NM_022105 | death associated transcription factor 1 | DATF1 |
| AW204564 | HCF-binding transcription factor Zhangfei | ZF |
| NM_003194 | TATA box binding protein | TBP |
| NM_005542 | insulin induced gene 1 | INSIG1 |
| AL540260 | hypothetical protein MGC17330 | MGC17330 |
| M60485 | “fibroblast growth factor receptor 1 (fms-related | FGFR1 |
| | tyrosine kinase 2, Pfeiffer syndrome)” | |
| AW270932 | hypothetical protein FLJ20275 | FLJ20275 |
| NM_020408 | CGI-203 protein | CGI-203 |
| BE675139 | hypothetical protein DKFZp586F2423 | DKFZP586F2423 |
| AF082283 | B-cell CLL/lymphoma 10 | BCL10 |
| U90304 | iroquois homeobox protein 5 | IRX5 |
| U79283 | D site of albumin promoter (albumin D-box) | DBP |
| | binding protein | |
| NM_018383 | hypothetical protein FLJ11294 | FLJ11294 |
| NM_005766 | “FERM, RhoGEF (ARHGEF) and pleckstrin | FARP1 |
| | domain protein 1 (chondrocyte-derived)” | |
| NM_001065 | “tumor necrosis factor receptor superfamily, | TNFRSF1A |
| | member 1A” | |
| NM_005981 | sarcoma amplified sequence | SAS |
| AU147889 | “Homo sapiens cDNA FLJ14122 fis, clone | |
| | MAMMA1002033” | |
| NM_020657 | zinc finger protein 304 | ZNF304 |
| U08015 | “nuclear factor of activated T-cells, cytoplasmic, | NFATC1 |
| | calcineurin-dependent 1” | |
| NM_014962 | BTB (POZ) domain containing 3 | BTBD3 |
| NM_004456 | enhancer of zeste homolog 2 (Drosophila) | EZH2 |
| AK023816 | “Homo sapiens cDNA FLJ13754 fis, clone | |
| | PLACE3000362” | |
| AI567426 | “transducin-like enhancer of split 3 (E(sp1) | TLE3 |
| | homolog, Drosophila)” | |
| NM_003082 | “small nuclear RNA activating complex, | SNAPC1 |
| | polypeptide 1, 43 kDa” | |
| NM_005815 | Kruppel-type zinc finger (C2H2) | ZK1 |
| NM_002222 | “inositol 1,4,5-triphosphate receptor, type 1” | ITPR1 |
| AL046979 | “Homo sapiens cDNA FLJ32766 fis, clone | |
| | TESTI2001862” | |
| NM_002193 | “inhibin, beta B (activin AB beta polypeptide)” | INHBB |
| NM_001941 | desmocollin 3 | DSC3 |
| NM_003478 | cullin 5 | CUL5 |
| NM_005433 | v-yes-1 Yamaguchi sarcoma viral oncogene | YES1 |
| | homolog 1 | |
| NM_000147 | “fucosidase, alpha-L-1, tissue” | FUCA1 |
| AC004010 | “Homo sapiens cDNA FLJ35792 fis, clone | |
| | TESTI2005759” | |
| NM_005308 | G protein-coupled receptor kinase 5 | GPRK5 |
| NM_004415 | “desmoplakin (DPI, DPII)” | DSP |
| NM_024994 | hypothetical protein FLJ12595 | FLJ12595 |
| BC003105 | “protein tyrosine phosphatase type IVA, member | PTP4A3 |
| | 3” | |
| X96588 | RYK receptor-like tyrosine kinase | RYK |
| NM_005652 | telomeric repeat binding factor 2 | TERF2 |
| NM_016929 | chloride intracellular channel 5 | CLIC5 |
| U63332 | “Human super cysteine rich protein mRNA, | |
| | partial cds” | |
| NM_005842 | sprouty homolog 2 (Drosophila) | SPRY2 |
| BF001670 | ephrin-B2 | EFNB2 |
| NM_003507 | frizzled homolog 7 (Drosophila) | FZD7 |
| AF153820 | “potassium inwardly-rectifying channel, | KCNJ2 |
| | subfamily J, member 2” | |
| NM_007079 | “protein tyrosine phosphatase type IVA, member | PTP4A3 |
| | 3” | |
| NM_025113 | hypothetical protein FLJ21562 | FLJ21562 |
| H49382 | “ESTs, Weakly similar to hypothetical protein | |
| | FLJ20378 [Homo sapiens] [H. sapiens]” | |
| AF021834 | tissue factor pathway inhibitor (lipoprotein- | TFPI |
| | associated coagulation inhibitor) | |
| BF031829 | “Homo sapiens, clone IMAGE: 4242700, mRNA” | |
| NM_022898 | B-cell CLL/lymphoma 11B (zinc finger protein) | BCL11B |
| AB020717 | synaptojanin 1 | SYNJ1 |
| NM_005433 | v-yes-1 Yamaguchi sarcoma viral oncogene | YES1 |
| | homolog 1 | |
| AL538264 | transcriptional intermediary factor 1 | TIF1 |
| AK026161 | apyrase | SHAPY |
| NM_012447 | stromal antigen 3 | STAG3 |
| NM_001313 | collapsin response mediator protein 1 | CRMP1 |
| H16758 | erythropoietin receptor | EPOR |
| NM_004815 | PTPL1-associated RhoGAP 1 | PARG1 |
| NM_004049 | BCL2-related protein A1 | BCL2A1 |
| NM_015905 | transcriptional intermediary factor 1 | TIF1 |
| AB006624 | KIAA0286 protein | KIAA0286 |
| AL534702 | abhydrolase domain containing 3 | ABHD3 |
| NM_006822 | “RAB40B, member RAS oncogene family” | RAB40B |
| M60459 | erythropoietin receptor | EPOR |
| M16276 | “major histocompatibility complex, class II, DQ | HLA-DQB1 |
| | beta 1” | |
| AI129310 | hypothetical protein FLJ21562 | FLJ21562 |
| NM_022161 | baculoviral IAP repeat-containing 7 (livin) | BIRC7 |
| NM_002248 | “potassium intermediate/small conductance | KCNN1 |
| | calcium-activated channel, subfamily N, member | |
| | 1” | |
| U42349 | Putative prostate cancer tumor suppressor | N33 |
| AB020683 | KIAA0876 protein | KIAA0876 |
| X00452 | “major histocompatibility complex, class II, DQ | HLA-DQA1 |
| | alpha 1” | |
| NM_016588 | neuritin 1 | NRN1 |
| AV753028 | transducin (beta)-like 1X-linked | TBL1X |
| AI348094 | KIAA0882 protein | KIAA0882 |
| AL049313 | Homo sapiens mRNA; cDNA DKFZp564B076 | |
| | (from clone DKFZp564B076) | |
| L20433 | “POU domain, class 4, transcription factor 1” | POU4F1 |
| NM_018159 | nudix (nucleoside diphosphate linked moiety X)- | NUDT11 |
| | type motif 11 | |
| AI884858 | Putative prostate cancer tumor suppressor | N33 |
| AB028641 | SRY (sex determining region Y)-box 11 | SOX11 |
| NM_006602 | transcription factor-like 5 (basic helix-loop-helix) | TCFL5 |
| AI360875 | SRY (sex determining region Y)-box 11 | SOX11 |
| AI040163 | “calcium channel, voltage-dependent, beta 2 | CACNB2 |
| | subunit” | |
| AI871641 | KIAA0871 protein | KIAA0871 |
| NM_002307 | “lectin, galactoside-binding, soluble, 7 (galectin | LGALS7 |
| | 7)” | |
| AL161995 | neurturin | NRTN |
| AW157202 | SRY (sex determining region Y)-box 11 | SOX11 |

TABLE 8A
Top genes discriminating L-asparaginase resistant and sensitive
B-lineage ALL: Genes down-regulated in L-asparaginase resistant B-lineage ALL

| | | | R/S | NCBI Accession |
|---|---|---|---|---|
| Probe ID | Gene Name | Gene Symbol | ratio | Number |
| 208579_x_at | histone 1, H2bk | HIST1H2BK | 0.49 | NM_017445 |
| 209806_at | histone 1, H2bk | HIST1H2BK | 0.6 | BC000893 |
| 203521_s_at | endocrine regulator | ZFP318 | 0.54 | NM_014345 |
| 212070_at | G protein-coupled | GPR56 | 0.39 | AL554008 |
| | receptor 56 | | | |
| 201015_s_at | junction plakoglobin | JUP | 0.63 | NM_021991 |
| 203388_at | arrestin, beta 2 | ARRB2 | 0.57 | NM_004313 |
| 221760_at | mannosidase, alpha, | MAN1A1 | 0.39 | BG287153 |
| | class 1A, member 1 | | | |
| 221773_at | Homo sapiens cDNA: | HRC08686. | 0.35 | AW575374 |
| | FLJ22425 fis, clone | | | |
| 211559_s_at | cyclin G2 | CCNG2 | 0.63 | L49506 |
| 218333_at | CGI-101 protein | F-LAN-1 | 0.71 | NM_016041 |
| 218669_at | hypothetical protein | LOC57826 | 0.64 | NM_021183 |
| | similar to small G | | | |
| | proteins, especially | | | |
| | RAP- | | | |
| 203311_s_at | ADP-ribosylation factor 6 | ARF6 | 0.67 | M57763 |
| 203274_at | coagulation factor VIII- | F8A | 0.62 | NM_012151 |
| | associated (intronic | | | |
| | transcript) | | | |
| 212643_at | chromosome 14 open | C14orf32 | 0.74 | AI671747 |
| | reading frame 32 | | | |
| 217750_s_at | hypothetical protein | FLJ13855 | 0.69 | NM_023079 |
| | FLJ13855 | | | |
| 201156_s_at | RAB5C, member RAS | RAB5C | 0.71 | AF141304 |
| | oncogene family | | | |
| 201140_s_at | RAB5C, member RAS | RAB5C | 0.63 | NM_004583 |
| | oncogene family | | | |

TABLE 8B
Top genes discriminating L-asparaginase resistant and sensitive
B-lineage ALL: Genes up-regulated in L-asparaginase resistant B-lineage ALL

| | | | R/S | NCBI Accession |
|---|---|---|---|---|
| Probe ID | Gene Name | Gene Symbol | ratio | Number |
| 220306_at | hypothetical protein | FLJ20202 | 1.87 | NM_017709 |
| | FLJ20202 | | | |
| 205131_x_at | stem cell growth factor; | SCGF | 3.38 | NM_002975 |
| | lymphocyte secreted C- | | | |
| | type lectin | | | |
| 211709_s_at | stem cell growth factor; | SCGF | 2.81 | BC005810 |
| | lymphocyte secreted C- | | | |
| | type lectin | | | |
| 201163_s_at | insulin-like growth | IGFBP7 | 2.02 | NM_001553 |
| | factor binding protein 7 | | | |
| 212886_at | DKFZP434C171 protein | DKFZP434C171 | 2.18 | AL080169 |
| 202315_s_at | breakpoint cluster | BCR | 1.57 | NM_004327 |
| | region | | | |
| 211940_x_at | H3 histone, family 3A | H3F3A | 1.22 | BE869922 |
| 208755_x_at | H3 histone, family 3A | H3F3A | 1.27 | BF312331 |
| 213828_x_at | H3 histone, family 3A | H3F3A | 1.27 | AA477655 |
| 209602_s_at | GATA binding protein 3 | GATA3 | 6.54 | AI796169 |
| 209604_s_at | GATA binding protein 3 | GATA3 | 3.12 | BC003070 |
| 212481_s_at | tropomyosin 4 | TPM4 | 1.47 | AI214061 |
| 217807_s_at | glioma tumor suppressor | GLTSCR2 | 1.43 | NM_015710 |
| | candidate region gene 2 | | | |
| 200005_at | eukaryotic translation | EIF3S7 | 1.39 | NM_003753 |
| | initiation factor 3, | | | |
| | subunit 7 zeta | | | |
| 217719_at | eukaryotic translation | EIF3S6IP | 1.38 | NM_016091 |
| | initiation factor 3, | | | |
| | subunit 6 interacting pro | | | |
| 210501_x_at | large tumor suppressor, | | 1.32 | AF119846 |
| | Drosophilahomolog 1 | | | |
| 211623_s_at | fibrillarin | FBL | 1.5 | M30448 |
| 200024_at | ribosomal protein S5 | RPS5 | 1.46 | NM_001009 |
| 200651_at | guanine nucleotide | GNB2L1 | 1.27 | NM_006098 |
| | binding protein (G | | | |
| | protein) | | | |
| 200010_at | ribosomal protein L11 | RPL11 | 1.25 | NM_000975 |
| 213080_x_at | ribosomal protein L5 | RPL5 | 1.37 | BF214492 |
| 200034_s_at | ribosomal protein L6 | RPL6 | 1.35 | NM_000970 |
| 200715_x_at | ribosomal protein L13a | RPL13A | 1.42 | BC000514 |
| 200689_x_at | eukaryotic translation | EEF1G | 1.27 | NM_001404 |
| | elongation factor 1 | | | |
| | gamma | | | |
| 208692_at | ribosomal protein S3 | RPS3 | 1.25 | U14990 |
| 211927_x_at | similar to S22655 | | 1.23 | BE963164 |
| | translation elongation | | | |
| | factor eEF-1 gamma | | | |
| | chain | | | |
| 200089_s_at | ribosomal protein L4 | RPL4 | 1.3 | AI953886 |
| 200705_s_at | eukaryotic translation | EEF1B2 | 1.46 | NM_001959 |
| | elongation factor 1 beta 2 | | | |
| 215963_x_at | similar to S34195 | | 1.39 | Z98200 |
| | ribosomal protein L3, | | | |
| | cytosolic | | | |
| 201217_x_at | ribosomal protein L3 | RPL3 | 1.42 | NM_000967 |
| 211666_x_at | ribosomal protein L3 | RPL3 | 1.43 | L22453 |
| 211073_x_at | ribosomal protein L3 | RPL3 | 1.43 | BC006483 |
| 212039_x_at | ribosomal protein L3 | RPL3 | 1.38 | BG339228 |
| 214167_s_at | ribosomal protein, large, | RPLP0 | 1.51 | AA555113 |
| | P0 | | | |
| 217740_x_at | ribosomal protein L7a | RPL7A | 1.32 | NM_000972 |
| 201818_at | hypothetical protein | FLJ12443 | 1.85 | NM_024830 |
| | FLJ12443 | | | |
| 215115_x_at | neurotrophic tyrosine | NTRK3 | 1.39 | AI613045 |
| | kinase, receptor, type 3 | | | |

TABLE 8C
Genes discriminating L-asparaginase resistant and sensitive
B-lineage ALL to the 0.001 < p < 0.01 level by both T-test and Wilcoxon: Genes
down-regulated in L-asparaginase resistant B-lineage ALL

| NCBI Accession | | |
|---|---|---|
| Number | Gene Name | Gene Symbol |
| NM_003919 | “sarcoglycan, epsilon” | SGCE |
| NM_002356 | myristoylated alanine-rich protein kinase C | MARCKS |
| | substrate | |
| AI348094 | KIAA0882 protein | KIAA0882 |
| AL582836 | paternally expressed 10 | PEG10 |
| AI983115 | class I cytokine receptor | WSX1 |
| M68956 | myristoylated alanine-rich protein kinase C | MARCKS |
| | substrate | |
| NM_004443 | EphB3 | EPHB3 |
| NM_005682 | G protein-coupled receptor 56 | GPR56 |
| M37780 | platelet/endothelial cell adhesion molecule | PECAM1 |
| | (CD31 antigen) | |
| NM_001154 | annexin A5 | ANXA5 |
| AW469573 | mitogen inducible 2 | MIG2 |
| L20433 | “POU domain, class 4, transcription factor 1” | POU4F1 |
| NM_002507 | “nerve growth factor receptor (TNFR | NGFR |
| | superfamily, member 16)” | |
| AI814527 | a disintegrin and metalloproteinase domain 8 | ADAM8 |
| D55696 | legumain | LGMN |
| AW134608 | immunoglobulin heavy constant gamma 3 | IGHG3 |
| | (G3m marker) | |
| NM_014879 | G protein-coupled receptor 105 | GPR105 |
| AI718937 | hypothetical protein BC013764 | LOC115207 |
| U76376 | “harakiri, BCL2 interacting protein | HRK |
| | (contains only BH3 domain)” | |
| AF220509 | “TAF9-like RNA polymerase II, TATA box | TAF9L |
| | binding protein (TBP)-associated factor, | |
| | 31 kDa” | |
| BG287153 | “mannosidase, alpha, class 1A, member 1” | MAN1A1 |
| AW575374 | “ELK3, ETS-domain protein (SRF | ELK3 |
| | accessory protein 2)” | |
| NM_004429 | ephrin-B1 | EFNB1 |
| AF241787 | Mitochondrial Acyl-CoA Thioesterase | MT-ACT48 |
| AI817041 | G protein-coupled receptor | RDC1 |
| AL554008 | G protein-coupled receptor 56 | GPR56 |
| AI970898 | “Homo sapiens, clone IMAGE: 3833472, | |
| | mRNA” | |
| NM_002736 | “protein kinase, cAMP-dependent, | PRKAR2B |
| | regulatory, type II, beta” | |
| AF176039 | high mobility group AT-hook 1 | HMGA1 |
| AA551075 | hypothetical protein BC013764 | LOC115207 |
| AB035482 | basement membrane-induced gene | ICB-1 |
| NM_001552 | insulin-like growth factor binding protein 4 | IGFBP4 |
| NM_006602 | transcription factor-like 5 (basic helix-loop- | TCFL5 |
| | helix) | |
| AB020681 | KIAA0874 protein | KIAA0874 |
| NM_006237 | “POU domain, class 4, transcription factor 1” | POU4F1 |
| NM_001629 | arachidonate 5-lipoxygenase-activating | ALOX5AP |
| | protein | |
| AK021738 | expressed in activated T/LAK lymphocytes | LAK-4P |
| NM_002201 | interferon stimulated gene 20 kDa | ISG20 |
| NM_014345 | endocrine regulator | HRIHFB2436 |
| M15330 | “interleukin 1, beta” | IL1B |
| AI655015 | Homo sapiens mRNA; cDNA | |
| | DKFZp586F2224 (from clone | |
| | DKFZp586F2224) | |
| NM_002452 | nudix (nucleoside diphosphate linked | NUDT1 |
| | moiety X)-type motif 1 | |
| NM_001124 | adrenomedullin | ADM |
| AA149594 | TGFB inducible early growth response 2 | TIEG2 |
| U82277 | “leukocyte immunoglobulin-like receptor, | LILRA2 |
| | subfamily A (with TM domain), member 2” | |
| NM_000905 | neuropeptide Y | NPY |
| NM_016229 | cytochrome b5 reductase b5R.2 | LOC51700 |
| AW163148 | myristoylated alanine-rich protein kinase C | MARCKS |
| | substrate | |
| NM_001449 | four and a half LIM domains 1 | FHL1 |
| NM_005652 | telomeric repeat binding factor 2 | TERF2 |
| AF063002 | four and a half LIM domains 1 | FHL1 |
| AI801013 | holocytochrome c synthase (cytochrome c | HCCS |
| | heme-lyase) | |
| BC000229 | hypothetical protein MGC2488 | MGC2488 |
| AF220153 | four and a half LIM domains 1 | FHL1 |
| NM_005815 | Kruppel-type zinc finger (C2H2) | ZK1 |
| AF186779 | RalGDS-like gene | RGL |
| AB001733 | immunoglobulin lambda joining 3 | IGLJ3 |
| NM_017445 | “H2B histone family, member S” | H2BFS |
| NM_019045 | similar to rab11-binding protein | FLJ11116 |
| NM_004848 | basement membrane-induced gene | ICB-1 |
| AF098518 | four and a half LIM domains 1 | FHL1 |
| NM_005907 | “mannosidase, alpha, class 1A, member 1” | MAN1A1 |
| BC005127 | adipose differentiation-related protein | ADFP |
| NM_016256 | N-acetylglucosamine-1-phosphodiester | LOC51172 |
| | alpha-N-acetylglucosaminidase | |
| AJ251844 | | |
| NM_006932 | smoothelin | SMTN |
| NM_003644 | growth arrest-specific 7 | GAS7 |
| NM_002408 | “mannosyl (alpha-1,6-)-glycoprotein beta- | MGAT2 |
| | 1,2-N-acetylglucosaminyltransferase” | |
| AI718418 | “stress 70 protein chaperone, microsome- | STCH |
| | associated, 60 kDa” | |
| NM_004233 | “CD83 antigen (activated B lymphocytes, | CD83 |
| | immunoglobulin superfamily)” | |
| AV721430 | “transcription factor 7-like 2 (T-cell | TCF7L2 |
| | specific, HMG-box)” | |
| M85256 | | |
| AI073984 | interferon consensus sequence binding | ICSBP1 |
| | protein 1 | |
| NM_000594 | “tumor necrosis factor (TNF superfamily, | TNF |
| | member 2)” | |
| BF970829 | oxysterol binding protein-like 8 | OSBPL8 |
| NM_019597 | heterogeneous nuclear ribonucleoprotein H2 (H′) | HNRPH2 |
| NM_006948 | “stress 70 protein chaperone, microsome- | STCH |
| | associated, 60 kDa” | |
| NM_004313 | “arrestin, beta 2” | ARRB2 |
| BF732638 | likely ortholog of mouse variant | CSTF2T |
| | polyadenylation protein CSTF-64 | |
| AI659005 | “ESTs, Moderately similar to unnamed | |
| | protein product [Homo sapiens] | |
| | [H. sapiens]” | |
| NM_003806 | “harakiri, BCL2 interacting protein | HRK |
| | (contains only BH3 domain)” | |
| NM_024576 | hypothetical protein FLJ21079 | FLJ21079 |
| NM_014885 | anaphase-promoting complex subunit 10 | APC10 |
| NM_018422 | hypothetical protein DKFZp761K1423 | DKFZp761K1423 |
| AL050164 | “chromodomain protein, Y chromosome- | CDYL |
| | like” | |
| NM_006965 | zinc finger protein 24 (KOX 17) | ZNF24 |
| NM_019034 | “ras homolog gene family, member F (in | ARHF |
| | filopodia)” | |
| NM_014061 | APR-1 protein | MAGEH1 |
| AL523275 | “Homo sapiens calmodulin-I (CALM1) | |
| | mRNA, 3′UTR, partial sequence” | |
| U30894 | N-sulfoglucosamine sulfohydrolase | SGSH |
| | (sulfamidase) | |
| NM_000576 | “interleukin 1, beta” | IL1B |
| NM_006359 | “solute carrier family 9 (sodium/hydrogen | SLC9A6 |
| | exchanger), isoform 6” | |
| L78132 | “lectin, galactoside-binding, soluble, 8 | LGALS8 |
| | (galectin 8)” | |
| AL542571 | GrpE-like protein cochaperone | HMGE |
| BC004908 | hypothetical protein MGC4655 | MGC4655 |
| AB011152 | “centaurin, delta 1” | CENTD1 |
| L49506 | cyclin G2 | CCNG2 |
| NM_017918 | hypothetical protein FLJ20647 | FLJ20647 |
| NM_005230 | “ELK3, ETS-domain protein (SRF | ELK3 |
| | accessory protein 2)” | |
| NM_001774 | CD37 antigen | CD37 |
| AA775177 | “protein tyrosine phosphatase, receptor type, | PTPRE |
| | E” | |
| AI191118 | “ESTs, Moderately similar to cytokine | |
| | receptor-like factor 2; cytokine receptor | |
| | CRL2 precusor [Homo sapiens] | |
| | [H. sapiens]” | |
| NM_004583 | “RAB5C, member RAS oncogene family” | RAB5C |
| NM_002406 | “mannosyl (alpha-1,3-)-glycoprotein beta- | MGAT1 |
| | 1,2-N-acetylglucosaminyltransferase” | |
| NM_000177 | “gelsolin (amyloidosis, Finnish type)” | GSN |
| NM_000788 | deoxycytidine kinase | DCK |
| NM_002448 | msh homeo box homolog 1 (Drosophila) | MSX1 |
| NM_005842 | sprouty homolog 2 (Drosophila) | SPRY2 |
| NM_018466 | uncharacterized hematopoietic | MDS031 |
| | stem/progenitor cells protein MDS031 | |
| NM_000698 | arachidonate 5-lipoxygenase | ALOX5 |
| NM_021183 | “hypothetical protein similar to small G | LOC57826 |
| | proteins, especially RAP-2A” | |
| AI742626 | HIV-1 Rev binding protein | HRB |
| NM_012151 | coagulation factor VIII-associated (intronic | F8A |
| | transcript) | |
| NM_003864 | “sin3-associated polypeptide, 30 kDa” | SAP30 |
| BC000893 | histone family member | H2B/S |
| BF432873 | “proteasome (prosome, macropain) 26S | PSMD11 |
| | subunit, non-ATPase, 11” | |
| NM_003088 | “singed-like (fascin homolog, sea urchin) | SNL |
| | (Drosophila)” | |
| NM_002835 | “protein tyrosine phosphatase, non-receptor | PTPN12 |
| | type 12” | |
| NM_006804 | START domain containing 3 | STARD3 |
| NM_003730 | ribonuclease 6 precursor | RNASE6PL |
| M17955 | “major histocompatibility complex, class II, | HLA-DQB1 |
| | DQ beta 1” | |
| AI653730 | “Homo sapiens calmodulin-I (CALM1) | |
| | mRNA, 3′UTR, partial sequence” | |
| NM_024613 | phafin 2 | FLJ13187 |
| AA702701 | platelet/endothelial cell adhesion molecule | PECAM1 |
| | (CD31 antigen) | |
| BG495771 | “Homo sapiens cDNA FLJ11918 fis, clone | |
| | HEMBB1000272” | |
| U88964 | interferon stimulated gene 20 kDa | ISG20 |
| AV694732 | cullin 4B | CUL4B |
| BC000373 | amyloid beta (A4) precursor-like protein 2 | APLP2 |
| AL049923 | oxysterol binding protein-like 8 | OSBPL8 |
| X75208 | EphB3 | EPHB3 |
| NM_012447 | stromal antigen 3 | STAG3 |
| NM_006821 | peroxisomal long-chain acyl-coA | ZAP128 |
| | thioesterase | |
| AJ276888 | “Mdm2, transformed 3T3 cell double | MDM2 |
| | minute 2, p53 binding protein (mouse)” | |
| NM_020408 | CGI-203 protein | CGI-203 |
| AA758755 | “proteasome (prosome, macropain) activator | PSME3 |
| | subunit 3 (PA28 gamma; Ki)” | |
| NM_017773 | hypothetical protein FLJ20340 | FLJ20340 |
| Z24459 | | |
| NM_018169 | hypothetical protein FLJ10652 | FLJ10652 |
| BF677486 | “thymosin, beta, identified in neuroblastoma | TMSNB |
| | cells” | |
| Z25433 | | |
| NM_018037 | hypothetical protein FLJ10244 | FLJ10244 |
| BG427393 | amyloid beta (A4) precursor-like protein 2 | APLP2 |
| AI653730 | “Homo sapiens calmodulin-I (CALM1) | |
| | mRNA, 3′UTR, partial sequence” | |
| NM_021991 | junction plakoglobin | JUP |
| U15555 | “serine palmitoyltransferase, long chain | SPTLC2 |
| | base subunit 2” | |
| NM_024644 | hypothetical protein FLJ21802 | FLJ21802 |
| AF217963 | “melanoma antigen, family D, 1” | MAGED1 |
| NM_003524 | “H2B histone family, member J” | H2BFJ |
| NM_007106 | ubiquitin-like 3 | UBL3 |
| NM_001642 | amyloid beta (A4) precursor-like protein 2 | APLP2 |
| BC006390 | “mannosyl (alpha-1,6-)-glycoprotein beta- | MGAT2 |
| | 1,2-N-acetylglucosaminyltransferase” | |
| NM_006936 | SMT3 suppressor of mif two 3 homolog 1 | SMT3H1 |
| | (yeast) | |
| NM_014043 | DKFZP564O123 protein | DKFZP564O123 |
| NM_002814 | “proteasome (prosome, macropain) 26S | PSMD10 |
| | subunit, non-ATPase, 10” | |
| AL560017 | prohibitin | PHB |
| NM_014175 | mitochondrial ribosomal protein L15 | MRPL15 |
| NM_016185 | hematological and neurological expressed 1 | HN1 |
| NM_004508 | isopentenyl-diphosphate delta isomerase | IDI1 |
| AI608725 | “intercellular adhesion molecule 1 (CD54), | ICAM1 |
| | human rhinovirus receptor” | |
| NM_001186 | “BTB and CNC homology 1, basic leucine | BACH1 |
| | zipper transcription factor 1” | |
| NM_018094 | G1 to S phase transition 2 | GSPT2 |
| Y13786 | a disintegrin and metalloproteinase domain | ADAM19 |
| | 19 (meltrin beta) | |
| NM_003199 | transcription factor 4 | TCF4 |
| NM_020633 | V1R-like 1 | V1RL1 |
| NM_005496 | SMC4 structural maintenance of | SMC4L1 |
| | chromosomes 4-like 1 (yeast) | |
| U82276 | “leukocyte immunoglobulin-like receptor, | LILRA2 |
| | subfamily A (with TM domain), member 2” | |
| NM_006520 | t-complex-associated-testis-expressed 1-like | TCTE1L |
| NM_002484 | “nucleotide binding protein 1 (MinD | NUBP1 |
| | homolog, E.coli)” | |
| AI591354 | heterogeneous nuclear ribonucleoprotein F | HNRPF |
| M25269 | “ELK1, member of ETS oncogene family” | ELK1 |
| BE858180 | paternally expressed 10 | PEG10 |
| BG338532 | SMT3 suppressor of mif two 3 homolog 1 | SMT3H1 |
| | (yeast) | |
| NM_016041 | CGI-101 protein | F-LAN-1 |
| NM_005359 | “MAD, mothers against decapentaplegic | MADH4 |
| | homolog 4 (Drosophila)” | |
| AI361850 | arachidonate 5-lipoxygenase | ALOX5 |
| AF141304 | “RAB5C, member RAS oncogene family” | RAB5C |
| BE549732 | “ESTs, Weakly similar to replication | |
| | initiation region protein (60 kD); zinc finger | |
| | proten AP4 [Homo sapiens] [H. sapiens]” | |
| NM_023079 | hypothetical protein FLJ13855 | FLJ13855 |
| AI084226 | regulator of Fas-induced apoptosis | TOSO |
| AV713053 | hypothetical protein FLJ14547 | FLJ14547 |
| NM_016608 | ALEX1 protein | ALEX1 |
| BG111168 | chromosome 6 open reading frame 9 | C6orf9 |
| Z21533 | hematopoietically expressed homeobox | HHEX |
| BC005123 | “serine palmitoyltransferase, long chain | SPTLC2 |
| | base subunit 2” | |
| BC006454 | growth arrest-specific 7 | GAS7 |
| AI356398 | “zinc finger protein 36, C3H type-like 2” | ZFP36L2 |
| M57763 | ADP-ribosylation factor 6 | ARF6 |
| NM_001470 | “gamma-aminobutyric acid (GABA) B | GABBR1 |
| | receptor, 1” | |
| NM_024121 | family with sequence similarity 11 member B | FAM11B |
| AA676803 | “Homo sapiens, clone IMAGE: 5243069, | |
| | mRNA” | |
| AK026678 | stromal antigen 2 | STAG2 |
| NM_004354 | cyclin G2 | CCNG2 |
| BC000903 | high-mobility group box 2 | HMGB2 |
| NM_006561 | “CUG triplet repeat, RNA binding protein | CUGBP2 |
| | 2” | |
| N22468 | “MADS box transcription enhancer factor 2, | MEF2C |
| | polypeptide C (myocyte enhancer factor | |
| | 2C)” | |
| AF132362 | heterogeneous nuclear ribonucleoprotein H3 | HNRPH3 |
| | (2H9) | |
| AI927067 | “Homo sapiens cDNA FLJ11918 fis, clone | |
| | HEMBB1000272” | |
| BG397856 | “major histocompatibility complex, class II, | HLA-DQA1 |
| | DQ alpha 1” | |
| NM_006570 | Ras-related GTP-binding protein | RAGA |
| AF342815 | “lectin, galactoside-binding, soluble, 8 | LGALS8 |
| | (galectin 8)” | |
| AW134535 | cyclin G2 | CCNG2 |
| NM_001968 | eukaryotic translation initiation factor 4E | EIF4E |
| AI139569 | SWAP-70 protein | SWAP70 |
| NM_021183 | “hypothetical protein similar to small G | LOC57826 |
| | proteins, especially RAP-2A” | |
| BC004421 | nucleolar cysteine-rich protein | HSA6591 |
| BG252842 | hypothetical protein FLJ12619 | FLJ12619 |
| NM_005697 | secretory carrier membrane protein 2 | SCAMP2 |
| NM_006333 | nuclear DNA-binding protein | C1D |
| NM_005574 | LIM domain only 2 (rhombotin-like 1) | LMO2 |
| NM_001529 | hematopoietically expressed homeobox | HHEX |
| NM_014210 | ecotropic viral integration site 2A | EVI2A |
| NM_025205 | endothelial-derived gene 1 | EG1 |
| NM_005342 | high-mobility group box 3 | HMGB3 |
| AI984421 | GrpE-like protein cochaperone | HMGE |
| NM_002118 | “major histocompatibility complex, class II, | HLA-DMB |
| | DM beta” | |
| AF064605 | death effector domain containing | DEDD |
| NM_007267 | expressed in activated T/LAK lymphocytes | LAK-4P |
| NM_005638 | synaptobrevin-like 1 | SYBL1 |
| NM_002634 | prohibitin | PHB |
| AI131008 | thyroid hormone receptor interactor 3 | TRIP3 |
| AU118026 | “Homo sapiens cDNA FLJ11918 fis, clone | |
| | HEMBB1000272” | |
| AL110243 | SOCS box-containing WD protein SWiP-1 | WSB1 |
| BF433429 | “Homo sapiens cDNA FLJ11918 fis, clone | |
| | HEMBB1000272” | |
| AF114012 | “tumor necrosis factor (ligand) superfamily, | TNFSF13 |
| | member 13” | |
| M32577 | | |
| AA502643 | “tyrosine 3-monooxygenase/tryptophan 5- | YWHAE |
| | monooxygenase activation protein, epsilon | |
| | polypeptide” | |
| BC005147 | “FK506 binding protein 1A, 12 kDa” | FKBP1A |
| AK001899 | APG5 autophagy 5-like (S. cerevisiae) | APG5L |
| NM_017936 | hypothetical protein FLJ20707 | FLJ20707 |
| BG390445 | ubiquitin specific protease 10 | USP10 |
| AW165960 | “protein tyrosine phosphatase type IVA, | PTP4A1 |
| | member 1” | |
| AV728658 | KIAA0592 protein | KIAA0592 |
| NM_018648 | “nucleolar protein family A, member 3 | NOLA3 |
| | (H/ACA small nucleolar RNPs)” | |
| BC006456 | hypothetical protein FLJ10824 | FLJ10824 |
| U32645 | E74-like factor 4 (ets domain transcription | ELF4 |
| | factor) | |
| AK026674 | transcription factor 4 | TCF4 |
| NM_000100 | cystatin B (stefin B) | CSTB |
| M90686 | “HLA-G histocompatibility antigen, class I, | HLA-G |
| | G” | |
| NM_017917 | hypothetical protein FLJ20644 | FLJ20644 |
| NM_021242 | hypothetical protein STRAIT11499 | STRAIT11499 |
| NM_003336 | ubiquitin-conjugating enzyme E2A (RAD6 | UBE2A |
| | homolog) | |
| M29335 | | |
| BC005247 | isopentenyl-diphosphate delta isomerase | IDI1 |
| X72631 | “nuclear receptor subfamily 1, group D, | NR1D1 |
| | member 1” | |
| BE962615 | sorting nexin 3 | SNX3 |
| NM_024064 | hypothetical protein MGC5363 | MGC5363 |
| AI096477 | zinc finger protein 363 | ZNF363 |
| NM_006460 | HMBA-inducible | HIS1 |
| NM_007373 | soc-2 suppressor of clear homolog (C. elegans) | SHOC2 |
| NM_030915 | likely ortholog of mouse limb-bud and heart | LBH |
| | gene | |
| AK026913 | WIRE protein | WIRE |
| BE465032 | hypothetical protein FLJ12619 | FLJ12619 |
| NM_001654 | v-raf murine sarcoma 3611 viral oncogene | ARAF1 |
| | homolog 1 | |
| BC004130 | nuclear domain 10 protein | NDP52 |
| AI936769 | “FK506 binding protein 1A, 12 kDa” | FKBP1A |
| BF592782 | “Homo sapiens cDNA FLJ11918 fis, clone | |
| | HEMBB1000272” | |
| NM_018149 | hypothetical protein FLJ10587 | FLJ10587 |
| AF254088 | zinc finger protein 278 | ZNF278 |
| NM_003244 | TGFB-induced factor (TALE family | TGIF |
| | homeobox) | |
| AL035588 | transcription factor EB | TFEB |
| AK001029 | ubiquilin 2 | UBQLN2 |
| AK023289 | hypothetical protein P15-2 | P15-2 |
| NM_004162 | “RAB5A, member RAS oncogene family” | RAB5A |
| AI671747 | likely ortholog of mouse MAPK-interacting | MISS |
| | and spindle-stabilizing protein | |
| BC001002 | hypothetical protein DKFZp434N0650 | DKFZp434N0650 |
| NM_017698 | hypothetical protein FLJ22679 | FLJ22679 |
| NM_002106 | “H2A histone family, member Z” | H2AFZ |
| D13988 | GDP dissociation inhibitor 2 | GDI2 |
| NM_004124 | “glia maturation factor, beta” | GMFB |
| AF001212 | “proteasome (prosome, macropain) 26S | PSMD11 |
| | subunit, non-ATPase, 11” | |
| W48843 | sprouty homolog 4 (Drosophila) | SPRY4 |
| NM_003523 | “H2B histone family, member H” | H2BFH |
| U94831 | transmembrane 9 superfamily member 1 | TM9SF1 |
| AI671747 | likely ortholog of mouse MAPK-interacting | MISS |
| | and spindle-stabilizing protein | |
| AI984479 | “Homo sapiens cDNA FLJ33067 fis, clone | |
| | TRACH2000148, weakly similar to | |
| | POLY(A) POLYMERASE (EC 2.7.7.19)” | |
| AV726646 | SMT3 suppressor of mif two 3 homolog 2 | SMT3H2 |
| | (yeast) | |
| NM_003131 | serum response factor (c-fos serum response | SRF |
| | element-binding transcription factor) | |
| NM_005736 | “ARP1 actin-related protein 1 homolog A, | ACTR1A |
| | centractin alpha (yeast)” | |
| NM_002494 | “NADH dehydrogenase (ubiquinone) 1, | NDUFC1 |
| | subcomplex unknown, 1, 6 kDa” | |
| Z25435 | | |
| NM_003591 | cullin 2 | CUL2 |
| NM_016129 | COP9 constitutive photomorphogenic | COPS4 |
| | homolog subunit 4 (Arabidopsis) | |
| NM_005428 | vav 1 oncogene | VAV1 |
| NM_018950 | “major histocompatibility complex, class I, | HLA-F |
| | F” | |
| AI768845 | synaptophysin-like protein | SYPL |
| NM_006432 | “Niemann-Pick disease, type C2” | NPC2 |
| AF279372 | “inositol 1,3,4-triphosphate 5/6 kinase” | ITPK1 |
| AW873564 | “ESTs, Weakly similar to YHS1_YEAST | |
| | HYPOTHETICAL 21.3 KD PROTEIN IN | |
| | MSH1-EPT1 INTERGENIC REGION | |
| | [S. cerevisiae]” | |
| NM_006255 | “protein kinase C, eta” | PRKCH |
| NM_016079 | CGI-149 protein | LOC51652 |
| NM_022107 | chromosome 6 open reading frame 9 | C6orf9 |
| M60334 | “major histocompatibility complex, class II, | HLA-DRA |
| | DR alpha” | |
| NM_002664 | pleckstrin | PLEK |
| AW139179 | fem-1 homolog b (C. elegans) | FEM1B |
| AF293841 | APG5 autophagy 5-like (S. cerevisiae) | APG5L |
| BC002809 | “down-regulator of transcription 1, TBP- | DR1 |
| | binding (negative cofactor 2)” | |
| AA524525 | zinc finger protein 363 | ZNF363 |
| NM_006667 | progesterone receptor membrane | PGRMC1 |
| | component 1 | |
| K03226 | “plasminogen activator, urokinase” | PLAU |
| NM_007278 | GABA(A) receptor-associated protein | GABARAP |
| BC000454 | “calmodulin 1 (phosphorylase kinase, | CALM1 |
| | delta)” | |
| NM_006923 | stromal cell-derived factor 2 | SDF2 |
| BC003133 | HLA-B associated transcript 3 | BAT3 |
| M27487 | “major histocompatibility complex, class II, | HLA-DPA1 |
| | DP alpha 1” | |
| AV702405 | emopamil binding protein (sterol isomerase) | EBP |
| NM_004639 | HLA-B associated transcript 3 | BAT3 |
| AF254087 | zinc finger protein 278 | ZNF278 |
| NM_021204 | E-1 enzyme | MASA |
| NM_002070 | “guanine nucleotide binding protein (G | GNAI2 |
| | protein), alpha inhibiting activity | |
| | polypeptide 2” | |
| NM_006754 | synaptophysin-like protein | SYPL |
| NM_005333 | holocytochrome c synthase (cytochrome c | HCCS |
| | heme-lyase) | |
| NM_006461 | mitotic spindle coiled-coil related protein | DEEPEST |
| NM_003096 | small nuclear ribonucleoprotein polypeptide G | SNRPG |
| AY007098 | hypothetical protein FLJ22405 | FLJ22405 |
| NM_003252 | TIA1 cytotoxic granule-associated RNA | TIAL1 |
| | binding protein-like 1 | |
| BG250310 | “zinc finger protein 36, C3H type-like 1” | ZFP36L1 |
| NM_031298 | hypothetical protein MGC2963 | MGC2963 |
| L76416 | SMT3 suppressor of mif two 3 homolog 2 | SMT3H2 |
| | (yeast) | |
| NM_014462 | Lsm1 protein | LSM1 |
| NM_016542 | Mst3 and SOK1-related kinase | MST4 |
| AL080183 | KIAA0592 protein | KIAA0592 |
| D80006 | disco-interacting protein 2 (Drosophila) | DIP2 |
| | homolog | |
| NM_020232 | hepatocellular carcinoma susceptibility | HCCA3 |
| | protein | |
| NM_004873 | BCL2-associated athanogene 5 | BAG5 |
| AF113514 | monocytic leukemia zinc finger protein- | MORF |
| | related factor | |
| BF718636 | “H2A histone family, member Z” | H2AFZ |
| BF448062 | “ubiquitin-conjugating enzyme E2D 3 | UBE2D3 |
| | (UBC4/5 homolog, yeast)” | |
| NM_014045 | low density lipoprotein receptor-related | LRP10 |
| | protein 10 | |
| AF135162 | cyclin I | CCNI |
| M27319 | “calmodulin 1 (phosphorylase kinase, | CALM1 |
| | delta)” | |
| S81916 | “Phosphoglycerate kinase {alternatively | |
| | spliced} [human, phosphoglycerate kinase | |
| | deficient patient with episodes of muscl, | |
| | mRNA Partial Mutant, 307 nt]” | |
| AI589086 | Lysosomal-associated multispanning | LAPTM5 |
| | membrane protein-5 | |
| AL031133 | “ESTs, Highly similar to SM32_HUMAN | |
| | Ubiquitin-like protein SMT3B (Sentrin 2) | |
| | [H. sapiens]” | |
| NM_004890 | sperm associated antigen 7 | SPAG7 |
| NM_003663 | CGG triplet repeat binding protein 1 | CGGBP1 |
| AK000818 | hypothetical protein FLJ20811 | FLJ20811 |
| BC001465 | HBS1-like (S. cerevisiae) | HBS1L |
| NM_021970 | mitogen-activated protein kinase kinase 1 | MAP2K1IP1 |
| | interacting protein 1 | |
| M80469 | | |
| M60333 | “major histocompatibility complex, class II, | HLA-DRA |
| | DR alpha” | |
| BG028844 | HLA-B associated transcript 3 | BAT3 |
| NM_007049 | “butyrophilin, subfamily 2, member A1” | BTN2A1 |
| AW514210 | “ESTs, Highly similar to A60384 MHC | |
| | class I histocompatibility antigen HLA-F | |
| | alpha chain Dew3 precursor - human | |
| | [H. sapiens]” | |
| AK024823 | SMT3 suppressor of mif two 3 homolog 2 | SMT3H2 |
| | (yeast) | |

TABLE 8D
Genes discriminating L-asparaginase resistant and sensitive
B-lineage ALL to the 0.001 < p < 0.01 level by both T-test and Wilcoxon: Genes
up-regulated in L-asparaginase resistant B-lineage ALL

| NCBI Accession | | |
|---|---|---|
| Number | Gene Name | Gene Symbol |
| L05095 | hypothetical protein FLJ22875 | FLJ22875 |
| NM_001015 | ribosomal protein S11 | RPS11 |
| NM_002136 | heterogeneous nuclear ribonucleoprotein A1 | HNRPA1 |
| BC004334 | ribosomal protein S10 | RPS10 |
| AI200589 | ribosomal protein S16 | RPS16 |
| NM_001686 | “ATP synthase, H+ transporting, | ATP5B |
| | mitochondrial F1 complex, beta | |
| | polypeptide” | |
| AF348700 | ubiquitin A-52 residue ribosomal protein | UBA52 |
| | fusion product 1 | |
| AA961748 | ribosomal protein L13 | RPL13 |
| NM_005548 | lysyl-tRNA synthetase | KARS |
| AI955655 | “H3 histone, family 3A” | H3F3A |
| BC000524 | ribosomal protein S6 | RPS6 |
| NM_000979 | ribosomal protein L18 | RPL18 |
| NM_001997 | Finkel-Biskis-Reilly murine sarcoma virus | FAU |
| | (FBR-MuSV) ubiquitously expressed (fox | |
| | derived); ribosomal protein S30 | |
| AA630314 | ribosomal protein S2 | RPS2 |
| NM_001010 | ribosomal protein S6 | RPS6 |
| AW574664 | ribosomal protein L13 | RPL13 |
| BC000354 | ribosomal protein S28 | RPS28 |
| NM_000973 | ribosomal protein L8 | RPL8 |
| BF247371 | hypothetical protein PRO1843 | PRO1843 |
| BC001675 | ribosomal protein L13a | RPL13A |
| NM_001004 | “ribosomal protein, large P2” | RPLP2 |
| BE968801 | ribosomal protein L35a | RPL35A |
| NM_001014 | ribosomal protein S10 | RPS10 |
| BC004886 | ribosomal protein S17 | RPS17 |
| BF979419 | “ESTs, Highly similar to R13A_HUMAN | |
| | 60S ribosomal protein L13a (23 kDa highly | |
| | basic protein) [H. sapiens]” | |
| AI560573 | ribosomal protein L24 | RPL24 |
| AA877641 | “adaptor-related protein complex 2, alpha 2 | AP2A2 |
| | subunit” | |
| BG152979 | ribosomal protein L22 | RPL22 |
| BE259729 | ribosomal protein S19 | RPS19 |
| NM_003640 | “inhibitor of kappa light polypeptide gene | IKBKAP |
| | enhancer in B-cells, kinase complex- | |
| | associated protein” | |
| NM_001021 | ribosomal protein S17 | RPS17 |
| NM_022551 | ribosomal protein S18 | RPS18 |
| NM_000990 | ribosomal protein L27a | RPL27A |
| D17652 | ribosomal protein L22 | RPL22 |
| NM_001054 | “sulfotransferase family, cytosolic, 1A, | SULT1A2 |
| | phenol-preferring, member 2” | |
| BE869922 | “H3 histone, family 3A” | H3F3A |
| AI805587 | ribosomal protein S7 | RPS7 |
| NM_004544 | “NADH dehydrogenase (ubiquinone) 1 | NDUFA10 |
| | alpha subcomplex, 10, 42 kDa” | |
| AB055804 | prefoldin 5 | PFDN5 |
| BE963164 | “ESTs, Highly similar to S22655 translation | |
| | elongation factor eEF-1 gamma chain - | |
| | human [H. sapiens]” | |
| NM_007273 | repressor of estrogen receptor activity | REA |
| NM_001022 | ribosomal protein S19 | RPS19 |
| NM_000986 | ribosomal protein L24 | RPL24 |
| NM_000034 | “aldolase A, fructose-bisphosphate” | ALDOA |
| NM_001013 | ribosomal protein S9 | RPS9 |
| AF061832 | heterogeneous nuclear ribonucleoprotein M | HNRPM |
| AI004246 | eukaryotic translation elongation factor 2 | EEF2 |
| AI970731 | ribosomal protein S7 | RPS7 |
| AI613273 | chromodomain helicase DNA binding | CHD4 |
| | protein 4 | |
| U14990 | ribosomal protein S3 | RPS3 |
| BF942308 | ribosomal protein L13a | RPL13A |
| AA888388 | ribosomal protein S25 | RPS25 |
| BG389744 | ribosomal protein L7 | RPL7 |
| AL118502 | | |
| X74070 | basic transcription factor 3 | BTF3 |
| NM_000991 | ribosomal protein L28 | RPL28 |
| BE741754 | ribosomal protein S6 | RPS6 |
| NM_012423 | ribosomal protein L13a | RPL13A |
| NM_015043 | KIAA0676 protein | KIAA0676 |
| AF322111 | KIAA1049 protein | KIAA1049 |
| AK026577 | “aldolase A, fructose-bisphosphate” | ALDOA |
| NM_000975 | ribosomal protein L11 | RPL11 |
| NM_003363 | ubiquitin specific protease 4 (proto- | USP4 |
| | oncogene) | |
| NM_016400 | Huntingtin interacting protein K | HYPK |
| NM_002624 | prefoldin 5 | PFDN5 |
| X89894 | “nuclear receptor subfamily 4, group A, | NR4A3 |
| | member 3” | |
| NM_016406 | hypothetical protein HSPC155 | HSPC155 |
| NM_001961 | eukaryotic translation elongation factor 2 | EEF2 |
| AA477655 | “H3 histone, family 3A” | H3F3A |
| BF312331 | “H3 histone, family 3A” | H3F3A |
| NM_006098 | “guanine nucleotide binding protein (G | GNB2L1 |
| | protein), beta polypeptide 2-like 1” | |
| NM_001012 | ribosomal protein S8 | RPS8 |
| AW304232 | “ESTs, Highly similar to RSP4_HUMAN | |
| | 40S ribosomal protein SA (P40) (34/67 kDa | |
| | laminin receptor) (Colon carcinoma | |
| | laminin-binding protein) (NEM/1CHD4) | |
| | [H. sapiens]” | |
| BC005863 | “ribosomal protein, large, P0” | RPLP0 |
| NM_007104 | ribosomal protein L10a | RPL10A |
| NM_005594 | nascent-polypeptide-associated complex | NACA |
| | alpha polypeptide | |
| NM_030662 | mitogen-activated protein kinase kinase 2 | MAP2K2 |
| NM_001404 | eukaryotic translation elongation factor 1 | EEF1G |
| | gamma | |
| AL530441 | “casein kinase 1, gamma 2” | CSNK1G2 |
| U78525 | “eukaryotic translation initiation factor 3, | EIF3S9 |
| | subunit 9 eta, 116 kDa” | |
| BC003655 | “ribosomal protein, large, P0” | RPLP0 |
| NM_001834 | “clathrin, light polypeptide (Lcb)” | CLTB |
| NM_001020 | ribosomal protein S16 | RPS16 |
| NM_007032 | Tara-like protein | HRIHFB2122 |
| NM_000968 | ribosomal protein L4 | RPL4 |
| R83000 | basic transcription factor 3 | BTF3 |
| NM_002954 | ribosomal protein S27a | RPS27A |
| BF431488 | DIPB protein | HSA249128 |
| BC006301 | anaphase promoting complex subunit 5 | ANAPC5 |
| NM_002096 | “general transcription factor IIF, | GTF2F1 |
| | polypeptide 1, 74 kDa” | |
| AK001196 | KIAA0676 protein | KIAA0676 |
| NM_005886 | katanin p80 (WD40-containing) subunit B 1 | KATNB1 |
| AV746402 | hypothetical protein MGC19556 | MGC19556 |
| NM_001018 | ribosomal protein S15 | RPS15 |
| NM_004597 | small nuclear ribonucleoprotein D2 | SNRPD2 |
| | polypeptide 16.5 kDa | |
| AF141870 | “interleukin enhancer binding factor 3, | ILF3 |
| | 90 kDa” | |
| AC004692 | | |
| AF119850 | | |
| NM_001002 | “ribosomal protein, large, P0” | RPLP0 |
| NM_015654 | DKFZP564C103 protein | DKFZP564C103 |
| AA838274 | ribosomal protein L14 | RPL14 |
| BE348997 | ribosomal protein S9 | RPS9 |
| AB014576 | KIAA0676 protein | KIAA0676 |
| N71116 | “phospholipase A2, group IVB (cytosolic)” | PLA2G4B |
| AI953822 | “ribosomal protein, large, P0” | RPLP0 |
| AB007931 | retinoblastoma-associated factor 600 | RBAF600 |
| BC005817 | ribosomal protein L4 | RPL4 |
| AB019691 | A kinase (PRKA) anchor protein (yotiao) 9 | AKAP9 |
| NM_000972 | ribosomal protein L7a | RPL7A |
| BC000120 | “general transcription factor IIF, | GTF2F1 |
| | polypeptide 1, 74 kDa” | |
| NM_003751 | “eukaryotic translation initiation factor 3, | EIF3S9 |
| | subunit 9 eta, 116 kDa” | |
| AL512760 | fatty acid desaturase 1 | FADS1 |
| AB011173 | KIAA0601 protein | KIAA0601 |
| NM_017802 | hypothetical protein FLJ20397 | FLJ20397 |
| NM_003169 | suppressor of Ty 5 homolog (S. cerevisiae) | SUPT5H |
| AI953886 | ribosomal protein L4 | RPL4 |
| U58766 | tissue specific transplantation antigen P35B | TSTA3 |
| AF001549 | | |
| U63131 | CDC37 cell division cycle 37 homolog (S. cerevisiae) | CDC37 |
| BG230614 | “CD47 antigen (Rh-related antigen, | CD47 |
| | integrin-associated signal transducer)” | |
| NM_001207 | basic transcription factor 3 | BTF3 |
| NM_024662 | hypothetical protein FLJ10774 | FLJ10774 |
| Y15521 | acetylserotonin O-methyltransferase-like | ASMTL |
| AK001684 | “ATPase, Ca++ transporting, type 2C, | ATP2C1 |
| | member 1” | |
| AF091085 | serologically defined breast cancer antigen | SDBCAG84 |
| | 84 | |
| NM_021640 | chromosome 12 open reading frame 10 | C12orf10 |
| NM_006833 | “COP9 subunit 6 (MOV34 homolog, 34 kD)” | MOV34-34KD |
| NM_000969 | ribosomal protein L5 | RPL5 |
| NM_004768 | “splicing factor, arginine/serine-rich 11” | SFRS11 |
| NM_001770 | CD19 antigen | CD19 |
| NM_002967 | scaffold attachment factor B | SAFB |
| NM_003756 | “eukaryotic translation initiation factor 3, | EIF3S3 |
| | subunit 3 gamma, 40 kDa” | |
| NM_024733 | hypothetical protein FLJ14345 | FLJ14345 |
| BC000478 | heat shock 70 kDa protein 9B (mortalin-2) | HSPA9B |
| L48784 | | |
| BF976260 | nascent-polypeptide-associated complex | NACA |
| | alpha polypeptide | |
| NM_001273 | chromodomain helicase DNA binding | CHD4 |
| | protein 4 | |
| BG339228 | ribosomal protein L3 | RPL3 |
| NM_031213 | “hypothetical protein MGC: 5244,” | MGC |
| NM_002743 | protein kinase C substrate 80K-H | PRKCSH |
| NM_000970 | ribosomal protein L6 | RPL6 |
| AF085358 | muscle specific gene | M9 |
| AF116273 | BCL2-associated athanogene | BAG1 |
| BC000580 | putative proline 4-hydroxylase | PH-4 |
| Z98200 | “ESTs, Weakly similar to RL3_HUMAN | |
| | 60S ribosomal protein L3 (HIV-1 TAR | |
| | RNA binding protein B) (TARBP-B) | |
| | [H. sapiens]” | |
| AY024365 | integrin-linked kinase-associated | ILKAP |
| | serine/threonine phosphatase 2C | |
| AB007457 | TP53 target gene 1 | TP53TG1 |
| NM_013265 | chromosome 11 open reading frame2 | C11orf2 |
| AF102988 | “phospholipase A2, group VI (cytosolic, | PLA2G6 |
| | calcium-independent)” | |
| BF214492 | ribosomal protein L5 | RPL5 |
| NM_013417 | isoleucine-tRNA synthetase | IARS |
| AC005011 | | |
| BC001689 | “solute carrier family 25 | SLC25A20 |
| | (carnitine/acylcarnitine translocase), | |
| | member 20” | |
| AA191576 | “nucleophosmin (nucleolar phosphoprotein | NPM1 |
| | B23, numatrin)” | |
| AK026096 | KIAA0676 protein | KIAA0676 |
| NM_003812 | a disintegrin and metalloproteinase domain | ADAM23 |
| | 23 | |
| AF119846 | muscle specific gene | M9 |
| NM_017724 | leucine rich repeat (in FLII) interacting | LRRFIP2 |
| | protein 2 | |
| BE737030 | “chaperonin containing TCP1, subunit 6A | CCT6A |
| | (zeta 1)” | |
| AL043112 | hypermethylated in cancer 2 | HIC2 |
| AW083133 | muscle specific gene | M9 |
| AW245400 | tyrosyl-tRNA synthetase | YARS |
| NM_006876 | “UDP-GlcNAc: betaGal beta-1,3-N- | B3GNT6 |
| | acetylglucosaminyltransferase 6” | |
| AW582267 | ribosomal protein L29 | RPL29 |
| NM_000967 | ribosomal protein L3 | RPL3 |
| NM_018119 | hypothetical protein FLJ10509 | FLJ10509 |
| NM_018555 | zinc finger protein 331; zinc finger protein | ZNF361 |
| | 463 | |
| BC006483 | ribosomal protein L3 | RPL3 |
| T33068 | anaphase promoting complex subunit 5 | ANAPC5 |
| NM_015414 | ribosomal protein L36 | RPL36 |
| NM_003827 | “N-ethylmaleimide-sensitive factor | NAPA |
| | attachment protein, alpha” | |
| NM_016091 | “eukaryotic translation initiation factor 3, | EIF3S6IP |
| | subunit 6 interacting protein” | |
| NM_014649 | KIAA0138 gene product | KIAA0138 |
| NM_015710 | glioma tumor suppressor candidate region | GLTSCR2 |
| | gene 2 | |
| BG403834 | “Homo sapiens cDNA FLJ20717 fis, clone | |
| | HEP18380” | |
| L22453 | | |
| AI653608 | leucine-rich PPR-motif containing | LRPPRC |
| AI744084 | hypothetical protein FLJ20850 | FLJ20850 |
| BC006332 | “clathrin, light polypeptide (Lcb)” | CLTB |
| AF251059 | hypothetical protein DKFZp761P1010 | DKFZp761P1010 |
| NM_002826 | quiescin Q6 | QSCN6 |
| AF315951 | | |
| BC002799 | KIAA1115 protein | KIAA1115 |
| U90268 | cerebral cavernous malformations 1 | CCM1 |
| BC000514 | ribosomal protein L13a | RPL13A |
| NM_015909 | neuroblastoma-amplified protein | NAG |
| AK024909 | “Homo sapiens cDNA: FLJ21256 fis, clone | |
| | COL01402” | |
| U87954 | “Human erbB3 binding protein EBP1 | |
| | mRNA, complete cds” | |
| BE670928 | “MDN1, midasin homolog (yeast)” | MDN1 |
| BG029530 | jumonji homolog (mouse) | JMJ |
| U25804 | “caspase 4, apoptosis-related cysteine | CASP4 |
| | protease” | |
| BF246115 | “Homo sapiens, RNA helicase-related | |
| | protein, clone MGC: 32732 | |
| | IMAGE: 4041046, mRNA, complete cds” | |
| L12387 | sorcin | SRI |
| AV727381 | ubiquinol-cytochrome c reductase core | UQCRC2 |
| | protein II | |
| NM_001055 | “sulfotransferase family, cytosolic, 1A, | SULT1A1 |
| | phenol-preferring, member 1” | |
| AW451954 | adaptor-associated kinase 1 | AAK1 |
| AL558875 | splicing factor proline/glutamine rich | SFPQ |
| | (polypyrimidine tract binding protein | |
| | associated) | |
| BC003086 | hypothetical protein FLJ20011 | FLJ20011 |
| NM_003753 | “eukaryotic translation initiation factor 3, | EIF3S7 |
| | subunit 7 zeta, 66/67 kDa” | |
| NM_024656 | hypothetical protein FLJ22329 | FLJ22329 |
| L25275 | “sulfotransferase family, cytosolic, 1A, | SULT1A3 |
| | phenol-preferring, member 3” | |
| NM_002198 | interferon regulatory factor 1 | IRF1 |
| NM_015057 | KIAA0916 protein | KIAA0916 |
| NM_001009 | ribosomal protein S5 | RPS5 |
| NM_006360 | dendritic cell protein | GA17 |
| AB029290 | microtubule-actin crosslinking factor 1 | MACF1 |
| BG256504 | KIAA0220 protein | KIAA0220 |
| BE250348 | ribosomal protein L22 | RPL22 |
| NM_004445 | EphB6 | EPHB6 |
| NM_001959 | eukaryotic translation elongation factor 1 | EEF1B2 |
| | beta 2 | |
| NM_000544 | “transporter 2, ATP-binding cassette, sub- | TAP2 |
| | family B (MDR/TAP)” | |
| AW071997 | ribosomal protein L22 | RPL22 |
| BC002977 | mel transforming oncogene (derived from | MEL |
| | cell line NK14)-RAB8 homolog | |
| NM_022644 | chorionic somatomammotropin hormone 2 | CSH2 |
| NM_007170 | testis-specific kinase 2 | TESK2 |
| NM_013447 | “egf-like module containing, mucin-like, | EMR2 |
| | hormone receptor-like sequence 2” | |
| NM_000175 | glucose phosphate isomerase | GPI |
| AF060511 | “Homo sapiens cDNA: FLJ23602 fis, clone | |
| | LNG15735” | |
| BC001081 | anaphase promoting complex subunit 5 | ANAPC5 |
| BC002827 | tropomyosin 4 | TPM4 |
| NM_004417 | dual specificity phosphatase 1 | DUSP1 |
| AA406605 | weakly similar to glutathione peroxidase 2 | CL683 |
| NM_001910 | cathepsin E | CTSE |
| BE968833 | “spectrin, beta, non-erythrocytic 1” | SPTBN1 |
| NM_005051 | glutaminyl-tRNA synthetase | QARS |
| NM_006360 | dendritic cell protein | GA17 |
| BC002629 | B-cell CLL/lymphoma 7A | BCL7A |
| NM_006817 | chromosome 12 open reading frame 8 | C12orf8 |
| AK024034 | “Homo sapiens cDNA FLJ13972 fis, clone | |
| | Y79AA1001548, highly similar to | |
| | PHOSPHATIDYLINOSITOL 4-KINASE | |
| | ALPHA (EC 2.7.1.67)” | |
| NM_016527 | hydroxyacid oxidase 2 (long chain) | HAO2 |
| AA555113 | “ribosomal protein, large, P0” | RPLP0 |
| BE540552 | fatty acid desaturase 1 | FADS1 |
| BE042354 | lactate dehydrogenase B | LDHB |
| AA206161 | KIAA0182 protein | KIAA0182 |
| N80922 | UDP-glucuronic acid/UDP-N- | UGTREL7 |
| | acetylgalactosamine dual transporter | |
| NM_002194 | inositol polyphosphate-1-phosphatase | INPP1 |
| U80918 | “nuclear factor of activated T-cells, | NFATC1 |
| | cytoplasmic, calcineurin-dependent 1” | |
| AF006011 | “dishevelled, dsh homolog 1 (Drosophila)” | DVL1 |
| NM_002300 | lactate dehydrogenase B | LDHB |
| NM_018188 | hypothetical protein FLJ10709 | FLJ10709 |
| NM_003028 | “Homo sapiens cDNA FLJ38908 fis, clone | |
| | NT2NE2005358” | |
| AA156721 | activated leukocyte cell adhesion molecule | ALCAM |
| NM_024569 | hypothetical protein FLJ21047 | FLJ21047 |
| AA527238 | nucleoporin 98 kDa | NUP98 |
| AA443762 | “guanine nucleotide binding protein (G | GNB2L1 |
| | protein), beta polypeptide 2-like 1” | |
| NM_018170 | hypothetical protein FLJ10656 | FLJ10656 |
| NM_015216 | KIAA0433 protein | KIAA0433 |
| AW024383 | ribosomal protein S21 | RPS21 |
| NM_024615 | hypothetical protein FLJ21308 | FLJ21308 |
| AA845710 | F-box and WD-40 domain protein 3 | FBXW3 |
| AK002111 | karyopherin alpha 6 (importin alpha 7) | KPNA6 |
| NM_024075 | leukocyte receptor cluster (LRC) member 5 | LENG5 |
| NM_002971 | special AT-rich sequence binding protein 1 | SATB1 |
| | (binds to nuclear matrix/scaffold- | |
| | associating DNA's) | |
| NM_016594 | “FK506 binding protein 11, 19 kDa” | FKBP11 |
| NM_023925 | hypothetical protein FLJ22569 | FLJ22569 |
| NM_024537 | hypothetical protein FLJ12118 | FLJ12118 |
| U64898 | nardilysin (N-arginine dibasic convertase) | NRD1 |
| M29277 | melanoma cell adhesion molecule | MCAM |
| NM_002147 | homeo box B5 | HOXB5 |
| NM_006893 | ligatin | LGTN |
| NM_006680 | “malic enzyme 3, NADP(+)-dependent, | ME3 |
| | mitochondrial” | |
| NM_001778 | CD48 antigen (B-cell membrane protein) | CD48 |
| NM_013366 | anaphase-promoting complex subunit 2 | APC2 |
| BF676980 | “glutamate-cysteine ligase, catalytic | GCLC |
| | subunit” | |
| M30448 | | |
| NM_014038 | basic leucine zipper and W2 domains 2 | BZW2 |
| NM_017967 | hypothetical protein FLJ20850 | FLJ20850 |
| AF087481 | putative DNA/chromatin binding motif | PLU-1 |
| NM_024653 | hypothetical protein FLJ13902 | FLJ13902 |
| NM_014254 | transmembrane protein 5 | TMEM5 |
| NM_003105 | “sortilin-related receptor, L(DLR class) A | SORL1 |
| | repeats-containing” | |
| AI613045 | “neurotrophic tyrosine kinase, receptor, type | NTRK3 |
| | 3” | |
| NM_005013 | nucleobindin 2 | NUCB2 |
| AI214061 | tropomyosin 4 | TPM4 |
| NM_000366 | tropomyosin 1 (alpha) | TPM1 |
| BG476661 | cell division cycle 34 | CDC34 |
| NM_015367 | MIL1 protein | MIL1 |
| NM_004528 | microsomal glutathione S-transferase 3 | MGST3 |
| NM_014367 | “hypothetical protein, estradiol-induced” | E2IG5 |
| NM_007274 | brain acyl-CoA hydrolase | BACH |
| AI971258 | seven in absentia homolog 1 (Drosophila) | SIAH1 |
| NM_000512 | “galactosamine (N-acetyl)-6-sulfate | GALNS |
| | sulfatase (Morquio syndrome, | |
| | mucopolysaccharidosis type IVA)” | |
| BE792224 | distal-less homeo box 2 | DLX2 |
| NM_018373 | synaptojanin 2 binding protein | SYNJ2BP |
| AF181660 | myelin protein zero-like 1 | MPZL1 |
| NM_004327 | breakpoint cluster region | BCR |
| D89976 | 5-aminoimidazole-4-carboxamide | ATIC |
| | ribonucleotide formyltransferase/IMP | |
| | cyclohydrolase | |
| AK023938 | “Homo sapiens cDNA FLJ13876 fis, clone | |
| | THYRO1001401” | |
| AL161999 | cytoplasmic FMR1 interacting protein 2 | CYFIP2 |
| J02959 | leukotriene A4 hydrolase | LTA4H |
| AK026142 | ocular development-associated gene | ODAG |
| AF274935 | putative membrane protein | LOC54499 |
| AV705803 | Homo sapiens mRNA; cDNA | |
| | DKFZp667K0521 (from clone | |
| | DKFZp667K0521) | |
| NM_000884 | IMP (inosine monophosphate) | IMPDH2 |
| | dehydrogenase 2 | |
| NM_024852 | hypothetical protein FLJ12765 | FLJ12765 |
| NM_016055 | mitochondrial ribosomal protein L48 | MRPL48 |
| U03891 | “apolipoprotein B mRNA editing enzyme, | APOBEC3A |
| | catalytic polypeptide-like 3A” | |
| AL117405 | KIAA0673 protein | KIAA0673 |
| AB002363 | KIAA0365 gene product | KIAA0365 |
| NM_003570 | cytidine monophosphate-N- | CMAH |
| | acetylneuraminic acid hydroxylase (CMP- | |
| | N-acetylneuraminate monooxygenase) | |
| NM_020202 | Nit protein 2 | NIT2 |
| NM_005384 | “nuclear factor, interleukin 3 regulated” | NFIL3 |
| NM_017664 | hypothetical protein FLJ20093 | FLJ20093 |
| NM_020993 | B-cell CLL/lymphoma 7A | BCL7A |
| NM_006870 | destrin (actin depolymerizing factor) | DSTN |
| NM_017688 | hypothetical protein FLJ20150 | FLJ20150 |
| NM_007066 | “protein kinase (cAMP-dependent, | PKIG |
| | catalytic) inhibitor gamma” | |
| NM_024068 | hypothetical protein MGC2731 | MGC2731 |
| BF514079 | Kruppel-like factor 4 (gut) | KLF4 |
| NM_001674 | activating transcription factor 3 | ATF3 |
| AI817942 | zeta-chain (TCR) associated protein kinase | ZAP70 |
| | 70 kDa | |
| NM_014229 | “solute carrier family 6 (neurotransmitter | SLC6A11 |
| | transporter, GABA), member 11” | |
| NM_017864 | hypothetical protein FLJ20530 | FLJ20530 |
| BE964361 | melanoma cell adhesion molecule | MCAM |
| NM_006194 | paired box gene 9 | PAX9 |
| NM_015380 | CGI-51 protein | CGI-51 |
| NM_020070 | immunoglobulin lambda-like polypeptide 1 | IGLL1 |
| NM_018945 | phosphodiesterase 7B | PDE7B |
| NM_003573 | latent transforming growth factor beta | LTBP4 |
| | binding protein 4 | |
| NM_025092 | hypothetical protein FLJ22635 | FLJ22635 |
| NM_000658 | autoimmune regulator (automimmune | AIRE |
| | polyendocrinopathy candidiasis ectodermal | |
| | dystrophy) | |
| BC003164 | leukocyte receptor cluster (LRC) member 4 | LENG4 |
| NM_000170 | “glycine dehydrogenase (decarboxylating; | GLDC |
| | glycine decarboxylase, glycine cleavage | |
| | system protein P)” | |
| NM_018467 | uncharacterized hematopoietic | MDS032 |
| | stem/progenitor cells protein MDS032 | |
| D86957 | KIAA0202 protein | KIAA0202 |
| AL121891 | likely ortholog of mouse ubiquitin | UBCE7IP5 |
| | conjugating enzyme 7 interacting protein 5 | |
| NM_017709 | hypothetical protein FLJ20202 | FLJ20202 |
| NM_018419 | SRY (sex determining region Y)-box 18 | SOX18 |
| AA723370 | CGI-105 protein | LOC51011 |
| NM_005213 | cystatin A (stefin A) | CSTA |
| AI675173 | prostaglandin E receptor 4 (subtype EP4) | PTGER4 |
| AL021786 | integral membrane protein 2A | ITM2A |
| NM_001451 | forkhead box F1 | FOXF1 |
| NM_024830 | hypothetical protein FLJ12443 | FLJ12443 |
| NM_024749 | hypothetical protein FLJ12505 | FLJ12505 |
| AB002301 | KIAA0303 protein | KIAA0303 |
| NM_001553 | insulin-like growth factor binding protein 7 | IGFBP7 |
| L19185 | peroxiredoxin 2 | PRDX2 |
| NM_004935 | cyclin-dependent kinase 5 | CDK5 |
| AB002366 | KIAA0368 protein | KIAA0368 |
| AL080169 | DKFZP434C171 protein | DKFZP434C171 |
| AA897516 | prostaglandin E receptor 4 (subtype EP4) | PTGER4 |
| NM_006885 | AT-binding transcription factor 1 | ATBF1 |
| NM_018700 | tripartite motif-containing 36 | TRIM36 |
| NM_002923 | “regulator of G-protein signalling 2, 24 kDa” | RGS2 |
| BG165833 | fatty acid desaturase 1 | FADS1 |
| AP001745 | chromosome 21 open reading frame 25 | C21orf25 |
| AL137651 | Homo sapiens mRNA; cDNA | |
| | DKFZp434O0213 (from clone | |
| | DKFZp434O0213); partial cds | |
| BC005810 | stem cell growth factor; lymphocyte | SCGF |
| | secreted C-type lectin | |
| NM_001498 | “glutamate-cysteine ligase, catalytic | GCLC |
| | subunit” | |
| NM_021114 | “serine protease inhibitor, Kazal type, 2 | SPINK2 |
| | (acrosin-trypsin inhibitor)” | |
| NM_002922 | regulator of G-protein signalling 1 | RGS1 |
| NM_001174 | Rho GTPase activating protein 6 | ARHGAP6 |
| NM_018100 | hypothetical protein FLJ10466 | FLJ10466 |
| D86586 | stem cell growth factor; lymphocyte | SCGF |
| | secreted C-type lectin | |
| BC003070 | GATA binding protein 3 | GATA3 |
| NM_018027 | hypothetical protein FLJ10210 | FLJ10210 |
| AI524125 | protocadherin 9 | PCDH9 |
| NM_001197 | BCL2-interacting killer (apoptosis- | BIK |
| | inducing) | |
| NM_002975 | stem cell growth factor; lymphocyte | SCGF |
| | secreted C-type lectin | |
| S59049 | regulator of G-protein signalling 1 | RGS1 |
| NM_001673 | asparagine synthetase | ASNS |
| AB020626 | KIAA0819 protein | KIAA0819 |
| AF127764 | “calpain 3, (p94)” | CAPN3 |
| AB035266 | neurexin 2 | NRXN2 |
| NM_024426 | Wilms tumor 1 | WT1 |
| AW663712 | KIAA0754 protein | KIAA0754 |
| AV711904 | tudor repeat associator with PCTAIRE 2 | PCTAIRE2BP |
| AI978623 | KIAA0657 protein | KIAA0657 |
| AL080170 | BIA2 | BIA2 |
| NM_001553 | insulin-like growth factor binding protein 7 | IGFBP7 |
| AI796169 | GATA binding protein 3 | GATA3 |
| AI796169 | GATA binding protein 3 | GATA3 |

TABLE 9A
Top genes discriminating daunorubicin resistant and sensitive B-
lineage ALL: Genes down-regulated in daunorubicin resistant B-lineage

| | | | | NCBI |
|---|---|---|---|---|
| | | | | Accession |
| Probe ID | Gene Name | Gene Symbol | R/S ratio | Number |
| 205062_x_at | retinoblastoma binding | RBBP1 | 0.73 | NM_002892 |
| | protein 1 | | | |
| 200059_s_at | ras homolog gene family, | ARHA | 0.75 | BC001360 |
| | member A | | | |
| 205070_at | inhibitor of growth | ING3 | 0.76 | NM_019071 |
| | family, member 3 | | | |
| 202521_at | CCCTC-binding factor | CTCF | 0.71 | NM_006565 |
| | (zinc finger protein) | | | |
| 219119_at | LSM8 homolog, U6 | LSM8 | 0.70 | NM_016200 |
| | small nuclear RNA | | | |
| | associated | | | |
| 218438_s_at | endothelial-derived gene 1 | EG1 | 0.67 | NM_025205 |
| 202777_at | soc-2 suppressor of clear | SHOC2 | 0.67 | NM_007373 |
| | homolog (C. elegans) | | | |
| 213264_at | mitogen-activated protein | MAP3K12 | 0.40 | AW025150 |
| | kinase kinase kinase 12 | | | |
| 218010_x_at | chromosome 20 open | C20orf149 | 0.74 | NM_024299 |
| | reading frame 149 | | | |
| 222251_s_at | glucocorticoid | GMEB2 | 0.67 | AL133646 |
| | modulatory element | | | |
| | binding protein 2 | | | |
| 213061_s_at | hypothetical protein | LOC123803 | 0.58 | AA643304 |
| | LOC123803 | | | |
| 208735_s_at | conserved gene amplified | OS4 | 0.71 | AF022231 |
| | in osteosarcoma | | | |

TABLE 9B
Top genes discriminating daunorubicin resistant and sensitive B-
lineage ALL: Genes up-regulated in daunorubicin resistant B-lineage

| | | | | NCBI |
|---|---|---|---|---|
| | | Gene | R/S | Accession |
| Probe ID | Gene Name | Symbol | ratio | Number |
| 207452_s_at | contactin 5 | CNTN5 | 1.82 | NM_014361 |
| 210966_x_at | likely ortholog of | LARP | 1.27 | BC001460 |
| | mouse la related | | | |
| | protein | | | |
| 219737_s_at | protocadherin 9 | PCDH9 | 2.97 | AI524125 |
| 203887_s_at | thrombomodulin | THBD | 1.61 | NM_000361 |
| 201694_s_at | early growth | EGR1 | 1.83 | NM_001964 |
| | response 1 | | | |
| 204401_at | K intermediate/small | KCNN4 | 1.75 | NM_002250 |
| | conductance calcium- | | | |
| | activated channel | | | |
| 209429_x_at | eukaryotic translation | EIF2B4 | 1.49 | AF112207 |
| | initiation factor 2B, | | | |
| | subunit 4 delta | | | |
| 201184_s_at | chromodomain | CHD4 | 1.47 | NM_001273 |
| | helicase DNA | | | |
| | binding protein 4 | | | |
| 209953_s_at | CDC37 cell division | CDC37 | 1.39 | U63131 |
| | cycle 37 homolog | | | |
| | (S. cerevisiae) | | | |
| 220948_s_at | ATPase, Na+/K+ | ATP1A1 | 1.41 | NM_000701 |
| | transporting, alpha 1 | | | |
| | polypeptide | | | |

TABLE 9C
Genes discriminating daunorubicin resistant and sensitive B-lineage
ALL to the 0.001 < p < 0.01 level by both T-test and Wilcoxon: Genes up-
regulated in daunorubicin resistant B-lineage

| NCBI Accession | | Gene |
|---|---|---|
| Number | Gene Name | Symbol |
| NM_018042 | hypothetical protein FLJ10260 | FLJ10260 |
| D55696 | legumain | LGMN |
| NM_000165 | “gap junction protein, alpha 1, 43 kDa (connexin | GJA1 |
| | 43)” | |
| NM_002234 | “potassium voltage-gated channel, shaker-related | KCNA5 |
| | subfamily, member 5” | |
| NM_003919 | “sarcoglycan, epsilon” | SGCE |
| AK025206 | “Homo sapiens cDNA: FLJ21553 fis, clone | |
| | COL06329” | |
| NM_030953 | tigger transposable element derived 6 | TIGD6 |
| AF195953 | | |
| J04132 | “CD3Z antigen, zeta polypeptide (TiT3 | CD3Z |
| | complex)” | |
| BG340670 | immunoglobulin heavy constant mu | IGHM |
| AW025150 | mitogen-activated protein kinase kinase kinase 12 | MAP3K12 |
| AW168948 | stromal antigen 1 | STAG1 |
| NM_018455 | uncharacterized bone marrow protein BM039 | BM039 |
| Z39557 | Homo sapiens clone 23705 mRNA sequence | |
| NM_006640 | MLL septin-like fusion | MSF |
| AV694732 | cullin 4B | CUL4B |
| AB032261 | stearoyl-CoA desaturase (delta-9-desaturase) | SCD |
| NM_014791 | maternal embryonic leucine zipper kinase | MELK |
| NM_007246 | “kelch-like 2, Mayven (Drosophila)” | KLHL2 |
| AK026236 | nucleoporin 160 kDa | NUP160 |
| AA643304 | Homo sapiens mRNA; cDNA DKFZp666E058 | |
| | (from clone DKFZp666E058) | |
| AW272611 | thymopoietin | TMPO |
| BC000731 | synaptogyrin 1 | SYNGR1 |
| NM_002835 | “protein tyrosine phosphatase, non-receptor type | PTPN12 |
| | 12” | |
| AF151853 | preimplantation protein 3 | PREI3 |
| BG164064 | ubiquitin-conjugating enzyme E2 variant 1 | UBE2V1 |
| NM_015928 | androgen-induced prostate proliferative shutoff | AS3 |
| | associated protein | |
| AI718418 | “stress 70 protein chaperone, microsome- | STCH |
| | associated, 60 kDa” | |
| NM_006717 | spindlin | SPIN |
| NM_007106 | ubiquitin-like 3 | UBL3 |
| AI571798 | Rho GDP dissociation inhibitor (GDI) alpha | ARHGDIA |
| BG338532 | SMT3 suppressor of mif two 3 homolog 1 (yeast) | SMT3H1 |
| AW139179 | fem-1 homolog b (C. elegans) | FEM1B |
| AI817061 | “protein phosphatase 1, regulatory (inhibitor) | PPP1R12A |
| | subunit 12A” | |
| NM_025205 | endothelial-derived gene 1 | EG1 |
| AW006345 | “signal sequence receptor, alpha (translocon- | SSR1 |
| | associated protein alpha)” | |
| BG390306 | spinocerebellar ataxia 7 (olivopontocerebellar | SCA7 |
| | atrophy with retinal degeneration) | |
| NM_004583 | “RAB5C, member RAS oncogene family” | RAB5C |
| NM_001227 | “caspase 7, apoptosis-related cysteine protease” | CASP7 |
| AL133646 | glucocorticoid modulatory element binding | GMEB2 |
| | protein 2 | |
| AB033078 | sphingosine-1-phosphate lyase 1 | SGPL1 |
| NM_007373 | soc-2 suppressor of clear homolog (C. elegans) | SHOC2 |
| AK025348 | “Homo sapiens cDNA: FLJ21695 fis, clone | |
| | COL09653” | |
| AF032105 | spinocerebellar ataxia 7 (olivopontocerebellar | SCA7 |
| | atrophy with retinal degeneration) | |
| NM_016200 | U6 snRNA-associated Sm-like protein LSm8 | LOC51691 |
| NM_002358 | MAD2 mitotic arrest deficient-like 1 (yeast) | MAD2L1 |
| NM_006218 | “phosphoinositide-3-kinase, catalytic, alpha | PIK3CA |
| | polypeptide” | |
| NM_004830 | “cofactor required for Sp1 transcriptional | CRSP3 |
| | activation, subunit 3, 130 kDa” | |
| AI872408 | likely ortholog of mouse variant polyadenylation | CSTF2T |
| | protein CSTF-64 | |
| AF022231 | conserved gene amplified in osteosarcoma | OS4 |
| NM_014744 | “TBC1 domain family, member 5” | TBC1D5 |
| NM_002892 | retinoblastoma binding protein 1 | RBBP1 |
| J03263 | lysosomal-associated membrane protein 1 | LAMP1 |
| NM_006565 | CCCTC-binding factor (zinc finger protein) | CTCF |
| NM_005663 | Wolf-Hirschhorn syndrome candidate 2 | WHSC2 |
| BG289800 | “SWI/SNF related, matrix associated, actin | SMARCE1 |
| | dependent regulator of chromatin, subfamily e, | |
| | member 1” | |
| NM_015153 | PHD finger protein 3 | PHF3 |
| NM_005359 | “MAD, mothers against decapentaplegic homolog | MADH4 |
| | 4 (Drosophila)” | |
| AU146275 | zinc finger protein 161 | ZNF161 |
| NM_014628 | gene predicted from cDNA with a complete | KIAA0110 |
| | coding sequence | |
| AI671747 | likely ortholog of mouse MAPK-interacting and | MISS |
| | spindle-stabilizing protein | |
| NM_004203 | membrane-associated tyrosine- and threonine- | PKMYT1 |
| | specific cdc2-inhibitory kinase | |
| NM_015986 | cytokine receptor-like factor 3 | CRLF3 |
| BC000383 | Wilms' tumour 1-associating protein | WTAP |
| NM_014045 | low density lipoprotein receptor-related protein | LRP10 |
| | 10 | |
| BF001666 | Homo sapiens clone 23870 mRNA sequence | |
| NM_030969 | hypothetical protein MGC1223 | MGC1223 |
| AL079310 | high-mobility group protein 2-like 1 | HMG2L1 |
| NM_024299 | chromosome 20 open reading frame 149 | C20orf149 |
| AI769416 | “CCR4-NOT transcription complex, subunit 8” | CNOT8 |
| M80776 | “adrenergic, beta, receptor kinase 1” | ADRBK1 |
| NM_020232 | hepatocellular carcinoma susceptibility protein | HCCA3 |
| NM_006327 | translocase of inner mitochondrial membrane 23 | TIMM23 |
| | homolog (yeast) | |
| NM_018604 | WW domain-containing adapter with a coiled- | WAC |
| | coil region | |
| NM_019071 | “inhibitor of growth family, member 3” | ING3 |
| NM_024612 | hypothetical protein FLJ22060 | FLJ22060 |
| AI688580 | SRB7 suppressor of RNA polymerase B homolog | SURB7 |
| | (yeast) | |
| AL136629 | TSPY-like | TSPYL |
| NM_014892 | KIAA1116 protein | KIAA1116 |
| AL136598 | protein associated with PRK1 | AWP1 |
| BE465032 | hypothetical protein FLJ12619 | FLJ12619 |
| AI694023 | hypothetical protein DKFZp761F0118 | DKFZp761F0118 |
| AA045183 | glucocorticoid modulatory element binding | GMEB2 |
| | protein 2 | |
| AI199589 | chromosome 20 open reading frame 67 | C20orf67 |
| NM_004124 | “glia maturation factor, beta” | GMFB |
| AA643304 | Homo sapiens mRNA; cDNA DKFZp666E058 | |
| | (from clone DKFZp666E058) | |
| AA580004 | ADP-ribosylation factor 1 | ARF1 |
| BC000903 | high-mobility group box 2 | HMGB2 |
| AU157515 | CDC10 cell division cycle 10 homolog (S. cerevisiae) | CDC10 |
| BC006462 | ubiquitin-like 1 (sentrin) | UBL1 |
| Z25435 | | |
| NM_024738 | hypothetical protein FLJ21415 | FLJ21415 |
| AL534321 | DAZ associated protein 2 | DAZAP2 |
| AF305239 | homeodomain interacting protein kinase 3 | HIPK3 |
| NM_006826 | “tyrosine 3-monooxygenase/tryptophan 5- | YWHAQ |
| | monooxygenase activation protein, theta | |
| | polypeptide” | |
| NM_006323 | “SEC24 related gene family, member B (S. cerevisiae)” | SEC24B |
| NM_007234 | dynactin 3 (p22) | DCTN3 |
| BC001360 | “ras homolog gene family, member A” | ARHA |
| AU146596 | KIAA0553 protein | KIAA0553 |
| AA004757 | zinc finger protein 236 | ZNF236 |
| BF430956 | PHD finger protein 3 | PHF3 |
| BE962615 | sorting nexin 3 | SNX3 |
| U69546 | “CUG triplet repeat, RNA binding protein 2” | CUGBP2 |
| AA919115 | “RAB14, member RAS oncogene family” | RAB14 |
| NM_016014 | CGI-67 protein | LOC51104 |
| NM_020191 | mitochondrial ribosomal protein S22 | MRPS22 |
| AF062483 | sorting nexin 3 | SNX3 |
| N64681 | WD repeat endosomal protein | KIAA1449 |
| L76416 | SMT3 suppressor of mif two 3 homolog 2 (yeast) | SMT3H2 |
| NM_014764 | DAZ associated protein 2 | DAZAP2 |
| AF202092 | autophagy Apg3p/Aut1p-like | APG3 |
| BC005876 | “ATPase, H+ transporting, lysosomal 21 kDa, V0 | ATP6V0B |
| | subunit c”” | |
| NM_003769 | “splicing factor, arginine/serine-rich 9” | SFRS9 |
| AF135162 | cyclin I | CCNI |
| AL117620 | vitiligo-associated protein VIT-1 | VIT1 |
| BC001465 | HBS1-like (S. cerevisiae) | HBS1L |

TABLE 9D
Genes discriminating daunorubicin resistant and sensitive B-lineage
ALL to the 0.001 < p < 0.01 level by both T-test and Wilcoxon: Genes down-
regulated in daunorubicin resistant B-lineage

| NCBI Accession | | Gene |
|---|---|---|
| Number | Gene Name | Symbol |
| AL050217 | Homo sapiens mRNA; cDNA DKFZp586I0523 | |
| | (from clone DKFZp586I0523) | |
| NM_006392 | nucleolar protein 5A (56 kDa with KKE/D repeat) | NOL5A |
| AW082913 | SFRS protein kinase 1 | SRPK1 |
| NM_015953 | eNOS interacting protein | NOSIP |
| AI613273 | chromodomain helicase DNA binding protein 4 | CHD4 |
| NM_023935 | chromosome 20 open reading frame 116 | C20orf116 |
| AL110273 | “spectrin, alpha, non-erythrocytic 1 (alpha- | SPTAN1 |
| | fodrin)” | |
| AB007931 | retinoblastoma-associated factor 600 | RBAF600 |
| NM_001754 | runt-related transcription factor 1 (acute myeloid | RUNX1 |
| | leukemia 1; aml1 oncogene) | |
| AU144792 | “Homo sapiens cDNA FLJ10127 fis, clone | |
| | HEMBA1002973, moderately similar to CAMP- | |
| | DEPENDENT 3′,5′-CYCLIC | |
| | PHOSPHODIESTERASE 4B (EC 3.1.4.17)” | |
| BC001460 | KIAA0731 protein | KIAA0731 |
| AV738039 | KIAA0365 gene product | KIAA0365 |
| NM_001239 | cyclin H | CCNH |
| NM_005566 | lactate dehydrogenase A | LDHA |
| BC001686 | “methionine adenosyltransferase II, alpha” | MAT2A |
| NM_012237 | sirtuin silent mating type information regulation 2 | SIRT2 |
| | homolog 2 (S. cerevisiae) | |
| Y15521 | acetylserotonin O-methyltransferase-like | ASMTL |
| NM_015367 | MIL1 protein | MIL1 |
| NM_002162 | intercellular adhesion molecule 3 | ICAM3 |
| NM_007295 | “breast cancer 1, early onset” | BRCA1 |
| U63131 | CDC37 cell division cycle 37 homolog (S. cerevisiae) | CDC37 |
| NM_000701 | “ATPase, Na+/K+ transporting, alpha 1 | ATP1A1 |
| | polypeptide” | |
| NM_001273 | chromodomain helicase DNA binding protein 4 | CHD4 |
| NM_002820 | parathyroid hormone-like hormone | PTHLH |
| AA191576 | “nucleophosmin (nucleolar phosphoprotein B23, | NPM1 |
| | numatrin)” | |
| NM_007272 | chymotrypsin C (caldecrin) | CTRC |
| NM_014905 | glutaminase | GLS |
| L32185 | “solute carrier family 11 (proton-coupled divalent | SLC11A1 |
| | metal ion transporters), member 1” | |
| NM_000674 | adenosine A1 receptor | ADORA1 |
| BG484069 | “Fanconi anemia, complementation group A” | FANCA |
| AI819238 | “inhibitor of DNA binding 2, dominant negative | ID2 |
| | helix-loop-helix protein” | |
| AF125393 | “RAB27A, member RAS oncogene family” | RAB27A |
| AW050627 | “centaurin, alpha 1” | CENTA1 |
| AF112207 | translation initiation factor eIF-2b delta subunit | EIF2B |
| AF051782 | diaphanous homolog 1 (Drosophila) | DIAPH1 |
| AF157324 | similar to S. cerevisiae RER1 | RER1 |
| AV733950 | early growth response 1 | EGR1 |
| AB011126 | formin-binding protein 17 | FBP17 |
| AC004382 | hypothetical protein DKFZp434K046 | DKFZP434K046 |
| NM_001108 | “acylphosphatase 2, muscle type” | ACYP2 |
| NM_012323 | v-maf musculoaponeurotic fibrosarcoma | MAFF |
| | oncogene homolog F (avian) | |
| NM_006554 | metaxin 2 | MTX2 |
| NM_016202 | LDL induced EC protein | LOC51157 |
| BG251266 | FOS-like antigen 1 | FOSL1 |
| NM_001964 | early growth response 1 | EGR1 |
| BF061658 | “transforming growth factor, beta 2” | TGFB2 |
| NM_000247 | MHC class I polypeptide-related sequence A | MICA |
| NM_001544 | “intercellular adhesion molecule 4, Landsteiner- | ICAM4 |
| | Wiener blood group” | |
| NM_019076 | “UDP glycosyltransferase 1 family, polypeptide | UGT1A |
| | A cluster” | |
| NM_000361 | thrombomodulin | THBD |
| NM_003236 | “transforming growth factor, alpha” | TGFA |
| NM_004418 | dual specificity phosphatase 2 | DUSP2 |
| NM_002250 | “potassium intermediate/small conductance | KCNN4 |
| | calcium-activated channel, subfamily N, member | |
| | 4” | |
| NM_000641 | interleukin 11 | IL11 |
| NM_021153 | “cadherin 19, type 2” | CDH19 |
| NM_004049 | BCL2-related protein A1 | BCL2A1 |
| NM_014361 | contactin 5 | CNTN5 |
| NM_025092 | hypothetical protein FLJ22635 | FLJ22635 |
| BC003070 | GATA binding protein 3 | GATA3 |
| NM_000399 | “early growth response 2 (Krox-20 homolog, | EGR2 |
| | Drosophila)” | |
| NM_002207 | “integrin, alpha 9” | ITGA9 |
| AI524125 | protocadherin 9 | PCDH9 |
| AI796169 | GATA binding protein 3 | GATA3 |

TABLE 10A
Genes discriminating prednisolone resistant and sensitive ALL
B- and T-lineage ALL): Genes down-regulated in prednisolone resistant ALL

| | | | | NCBI |
|---|---|---|---|---|
| | | | R/S | Accession |
| Probe ID | Gene Name | Gene Symbol | ratio | Number |
| 212726_at | PHD finger protein 2 | PHF2 | 0.51 | AB014562 |
| 213061_s_at | hypothetical protein | LOC123803 | 0.68 | AA643304 |
| | LOC123803 | | | |
| 212167_s_at | SWI/SNF related, matrix | SMARCB1 | 0.82 | AK021419 |
| | associated, actin | | | |
| | dependent regulator | | | |
| 209675_s_at | E1B-55 kDa-associated | E1B-AP5 | 0.65 | BC004242 |
| | protein 5 | | | |
| 217729_s_at | amino-terminal enhancer | AES | 0.71 | NM_001130.3 |
| | of split | | | |
| 208620_at | poly(rC) binding protein 1 | PCBP1 | 0.68 | U24223 |
| 203274_at | coagulation factor VIII- | F8A | 0.61 | NM_012151 |
| | associated (intronic | | | |
| | transcript) | | | |
| 218438_s_at | endothelial-derived gene 1 | EG1 | 0.72 | NM_025205 |
| 210092_at | mago-nashi homolog, | MAGOH | 0.76 | AF067173 |
| | proliferation-associated | | | |
| | (Drosophila) | | | |
| 208739_x_at | SMT3 suppressor of mif two | SMT3H2 | 0.72 | L76416 |
| | 3 homolog 2 (yeast) | | | |
| 218381_s_at | U2 small nuclear | U2AF65 | 0.81 | NM_007279 |
| | ribonucleoprotein | | | |
| | auxiliary factor (65 kD) | | | |
| 210053_at | Homo sapiens mRNA; | | 0.54 | AW138827 |
| | cDNA DKFZp434I1626 | | | |
| 201821_s_at | translocase of inner | TIMM17A | 0.67 | BC004439 |
| | mitochondrial membrane | | | |
| | 17 homolog A | | | |
| 212180_at | v-crk sarcoma virus CT10 | CRKL | 0.74 | AK000311 |
| | oncogene homolog | | | |
| | (avian)-like | | | |
| 208765_s_at | heterogeneous nuclear | HNRPR | 0.70 | NM_005826 |
| | ribonucleoprotein R | | | |
| 38710_at | ubiquitin-specific protease | FLJ20113 | 0.81 | AL096714 |
| | otubain 1 | | | |
| 212100_s_at | KIAA1649 protein | KIAA1649 | 0.85 | Z93241 |
| 203521_s_at | endocrine regulator | ZFP318 | 0.49 | NM_014345 |
| 206865_at | harakiri, BCL2 interacting | HRK 0.30 | | U76376 |
| | protein (contains only | | | |
| | BH3 domain) | | | |
| 209760_at | KIAA0922 protein | KIAA0922 | 0.68 | AL136932 |

TABLE 10B
Genes discriminating prednisolone resistant and sensitive ALL (B-
and T-lineage ALL): Genes up-regulated in prednisolone resistant ALL

| | | | | NCBI |
|---|---|---|---|---|
| | | | | Accession |
| Probe ID | Gene Name | Gene Symbol | R/S ratio | Number |
| 206209_s_at | carbonic anhydrase IV | CA4 | 1.48 | NM_000717 |
| 210423_s_at | solute carrier family 11 | SLC11A1 | 1.41 | L32185 |
| 206267_s_at | megakaryocyte-associated | MATK | 2.18 | NM_002378 |
| | tyrosine kinase | | | |
| 206905_s_at | matrilin 1, cartilage matrix | MATN1 | 1.86 | NM_002379 |
| | protein | | | |
| 210140_at | cystatin F (leukocystatin) | CST7 | 2.21 | AF031824 |
| 207988_s_at | actin related protein 2/3 | ARPC2 | 1.25 | NM_005731 |
| | complex, subunit 2, | | | |
| | 34 kDa | | | |
| 201061_s_at | stomatin | STOM | 1.79 | M81635 |
| 214057_at | myeloid cell leukemia | MCL1 | 1.60 | H71805 |
| | sequence 1 (BCL2- | | | |
| | related) | | | |
| 216907_x_at | killer cell | KIR3DL2 | 1.32 | X93596 |
| | immunoglobulin-like | | | |
| | receptor | | | |
| 202771_at | KIAA0233 gene product | KIAA0233 | 2.17 | NM_014745 |
| 219380_x_at | polymerase (DNA | POLH | 1.89 | NM_006502 |
| | directed), eta | | | |
| 203139_at | death-associated protein | DAPK1 | 1.71 | NM_004938 |
| | kinase 1 | | | |

TABLE 11A
Genes discriminating vincristine resistant and sensitive ALL (B-
and T-lineage ALL): Genes down-regulated in vincristine resistant ALL

| | | | | NCBI |
|---|---|---|---|---|
| | | | R/S | Accession |
| Probe ID | Gene Name | Gene Symbol | ratio | Number |
| 212210_at | DKFZP586J0619 protein | DKFZP586J0619 | 0.47 | AB037861 |
| 200593_s_at | heterogeneous nuclear | HNRPU | 0.77 | BC003621 |
| | ribonucleoprotein U | | | |
| | (scaffold attachmen | | | |
| 217733_s_at | thymosin, beta 10 | TMSB10 | 0.87 | NM_021103 |
| 200809_x_at | ribosomal protein L12 | RPL12 | 0.84 | NM_000976 |
| 200088_x_at | cDNA (AK026491) | | 0.81 | AK026491 |
| 214271_x_at | ribosomal protein L12 | RPL12 | 0.83 | AA281332 |
| 200725_x_at | ribosomal protein L10 | RPL10 | 0.85 | NM_006013 |
| 200061_s_at | ribosomal protein S24 | RPS24 | 0.78 | BC000523 |
| 213084_x_at | ribosomal protein L23a | RPL23A | 0.81 | BF125158 |
| 208825_x_at | ribosomal protein L23a | RPL23A | 0.81 | U43701 |
| 208834_x_at | ribosomal protein L23a | RPL23A | 0.78 | BC001865 |
| 200963_x_at | ribosomal protein L31 | RPL31 | 0.82 | NM_000993 |
| 213377_x_at | complement component 1, | C1S | 0.83 | AI799007 |
| | s subcomponent | | | |
| 200781_s_at | ribosomal protein S15a | RPS15A | 0.80 | NM_001019 |
| 200909_s_at | ribosomal protein, large P2 | RPLP2 | 0.86 | NM_001004 |
| 212433_x_at | ribosomal protein S2 | RPS2 | 0.79 | AA630314 |
| 200680_x_at | high-mobility group box 1 | HMGB1 | 0.83 | NM_002128 |
| 201268_at | non-metastatic cells 2, | NME2 | 0.71 | NM_002512 |
| | protein (NM23B) | | | |
| | expressed in | | | |
| 204559_s_at | LSM7 homolog, U6 small | LSM7 | 0.76 | NM_016199 |
| | nuclear RNA associated | | | |
| 218608_at | putative ATPase | HSA9947 | 0.18 | NM_022089 |
| 214369_s_at | RAS guanyl releasing | RASGRP2 | 0.65 | AI688812 |
| | protein 2 (calcium and | | | |
| | DAG-regulated) | | | |
| 218672_at | hypothetical protein | MGC3180 | 0.75 | NM_024041 |
| | MGC3180 | | | |
| 204490_s_at | CD44 antigen (homing | CD44 | 0.43 | M24915 |
| | function and Indian blood group | | | |
| | system) | | | |
| 201426_s_at | ribosomal protein, large | RPLP2 | 0.58 | AI922599 |
| | P2 | | | |

TABLE 11B
Genes discriminating vincristine resistant and sensitive ALL (B-
and T-lineage ALL): Genes up-regulated in vincristine resistant ALL

| | | | | NCBI |
|---|---|---|---|---|
| | | | R/S | Accession |
| Probe ID | Gene Name | Gene Symbol | ratio | Number |
| 212335_at | glucosamine (N-acetyl)-6- | GNS | 1.45 | AW167793 |
| | sulfatase (Sanfilippo | | | |
| | disease IIID) | | | |
| 218647_s_at | hypothetical protein | FLJ23476 | 1.31 | NM_024640 |
| | FLJ23476 | | | |
| 213225_at | protein phosphatase 1B | PPM1B | 1.37 | AJ271832 |
| | (formerly 2C), magnesium- | | | |
| | dependent | | | |
| 201088_at | karyopherin alpha 2 (RAG | KPNA2 | 1.40 | NM_002266 |
| | cohort 1, importin alpha 1) | | | |
| 202392_s_at | phosphatidylserine | PISD | 1.26 | NM_014338 |
| | decarboxylase | | | |
| 213604_at | Homo sapiens clone 24582 | | 1.29 | AW451236 |
| | mRNA sequence | | | |
| 212049_at | Homo sapiens cDNA | | 1.19 | AK026913 |
| | FLJ30463 fis, clone | | | |
| | BRACE2009517. | | | |
| 221752_at | slingshot 1 | SSH1 | 1.19 | AB037719 |
| 200757_s_at | calumenin | CALU | 1.22 | NM_001219 |
| 215127_s_at | RNA binding motif, single | RBMS1 | 1.33 | AL517946 |
| | stranded interacting protein 1 | | | |
| 204434_at | spermatogenesis associated 2 | SPATA2 | 1.34 | NM_006038 |
| 55872_at | KIAA1196 protein | KIAA1196 | 1.45 | AI493119 |
| 210791_s_at | Rho GTPase-activating | RICS | 1.59 | BC000277 |
| | protein | | | |
| 215616_s_at | KIAA0876 protein | KIAA0876 | 2.72 | AB020683 |
| 212492_s_at | KIAA0876 protein | KIAA0876 | 1.45 | AW237172 |
| 210187_at | FK506 binding protein 1A, | FKBP1A | 1.35 | BC005147 |
| | 12 kDa | | | |
| 212345_s_at | hypothetical protein | DKFZP586F2423 | 1.47 | BE675139 |
| | DKFZp586F2423 | | | |
| 213301_x_at | transcriptional | TIF1 | 2.46 | AL538264 |
| | intermediary factor 1 | | | |
| 204391_x_at | transcriptional | TIF1 | 2.56 | NM_015905 |
| | intermediary factor 1 | | | |
| 37986_at | erythropoietin receptor | EPOR | 2.19 | M60459 |
| 212438_at | putative nucleic acid | RY1 | 1.40 | BG252325 |
| | binding protein RY-1 | | | |
| 213939_s_at | Homo sapiens cDNA | | 3.63 | AI871641 |
| | FLJ12012 fis, clone | | | |
| | HEMBB1001668. | | | |
| 213017_at | abhydrolase domain | ABHD3 | 1.93 | AL534702 |
| | containing 3 | | | |
| 221011_s_at | likely ortholog of mouse | LBH | 1.45 | NM_030915 |
| | limb-bud and heart gene | | | |
| 44790_s_at | chromosome 13 open | C13orf18 | 2.06 | AI129310 |
| | reading frame 18 | | | |
| 206574_s_at | protein tyrosine | PTP4A3 | 2.10 | NM_007079 |
| | phosphatase type IVA, | | | |
| | member 3 | | | |
| 209695_at | protein tyrosine | PTP4A3 | 1.69 | BC003105 |
| | phosphatase type IVA, | | | |
| | member 3 | | | |
| 206033_s_at | desmocollin 3 | DSC3 | 1.71 | NM_001941 |
| 206231_at K | intermediate/small | KCNN1 | 1.65 | NM_002248 |
| | conductance calcium- | | | |
| | activated channel | | | |
| 208056_s_at | core-binding factor, runt | CBFA2T3 | 1.50 | NM_005187 |
| | domain, alpha subunit 2; | | | |
| | translocated t | | | |

TABLE 12A
Genes discriminating L-asparaginase resistant and sensitive ALL
(B- and T-lineage ALL): Genes down-regulated in L-asparaginase resistant
ALL

| | | | | NCBI |
|---|---|---|---|---|
| | | | | Accession |
| Probe ID | Gene Name | Gene Symbol | R/S ratio | Number |
| 217602_at | peptidylprolyl isomerase | PPIA | 0.66 | AI191118 |
| | A (cyclophilin A) | | | |
| 208656_s_at | cyclin I | CCNI | 0.85 | AF135162 |
| 218802_at | hypothetical protein | FLJ20647 | 0.65 | NM_017918 |
| | FLJ20647 | | | |
| 203388_at | arrestin, beta 2 | ARRB2 | 0.70 | NM_004313 |
| 202557_at | stress 70 protein | STCH | 0.68 | AI718418 |
| | chaperone, microsome- | | | |
| | associated | | | |
| 216862_s_at | mature T-cell | MTCP1 | 0.72 | Z24459 |
| | proliferation 1 | | | |
| 218027_at | mitochondrial ribosomal | MRPL15 | 0.69 | NM_014175 |
| | protein L15 | | | |
| 203094_at | gene predicted from | CMT2 | 0.78 | NM_014628 |
| | cDNA with a complete | | | |
| | coding sequence | | | |
| 201668_x_at | myristoylated alanine- | MARCKS | 0.53 | AW163148 |
| | rich protein kinase C | | | |
| | substrate | | | |
| 201670_s_at | myristoylated alanine- | MARCKS | 0.27 | M68956 |
| | rich protein kinase C | | | |
| | substrate | | | |
| 201669_s_at | myristoylated alanine- | MARCKS | 0.20 | NM_002356 |
| | rich protein kinase C | | | |
| | substrate | | | |
| 211255_x_at | death effector domain | DEDD | 0.81 | AF064605 |
| | containing | | | |
| 203521_s_at | endocrine regulator | ZFP318 | 0.58 | NM_014345 |
| 218614_at | hypothetical protein | FLJ10652 | 0.72 | NM_018169 |
| | FLJ10652 | | | |
| 212749_s_at | zinc finger protein 363 | ZNF363 | 0.77 | AL050144 |
| 211559_s_at | cyclin G2 | CCNG2 | 0.67 | L49506 |
| 202769_at | cyclin G2 | CCNG2 | 0.73 | AW134535 |
| 200987_x_at | proteasome (prosome, | PSME3 | 0.74 | AA758755 |
| | macropain) activator | | | |
| | subunit 3 | | | |
| 201376_s_at | heterogeneous nuclear | HNRPF | 0.74 | AI591354 |
| | ribonucleoprotein F | | | |
| 201140_s_at | RAB5C, member RAS | RAB5C | 0.72 | NM_004583 |
| | oncogene family | | | |
| 217750_s_at | hypothetical protein | FLJ13855 | 0.75 | NM_023079 |
| | FLJ13855 | | | |
| 203311_s_at | ADP-ribosylation factor 6 | ARF6 | 0.67 | M57763 |
| 212643_at | chromosome 14 open | C14orf32 | 0.78 | AI671747 |
| | reading frame 32 | | | |
| 218333_at | CGI-101 protein | F-LAN-1 | 0.74 | NM_016041 |
| 218438_s_at | endothelial-derived gene 1 | EG1 | 0.79 | NM_025205 |
| 203274_at | coagulation factor VIII- | F8A | 0.70 | NM_012151 |
| | associated (intronic | | | |
| | transcript) | | | |
| 212544_at | thyroid hormone receptor | TRIP3 | 0.82 | AI131008 |
| | interactor 3 | | | |
| 205644_s_at | small nuclear | SNRPG | 0.74 | NM_003096 |
| | ribonucleoprotein | | | |
| | polypeptide G | | | |
| 208739_x_at | SMT3 suppressor of mif | SMT3H2 | 0.81 | L76416 |
| | two 3 homolog 2 (yeast) | | | |

TABLE 12B
Genes discriminating L-asparaginase resistant and sensitive ALL
(B- and T-lineage ALL): Genes up-regulated in L-asparaginase resistant ALL

| | | | | NCBI |
|---|---|---|---|---|
| | | | | Accession |
| Probe ID | Gene Name | Gene Symbol | R/S ratio | Number |
| 212792_at | KIAA0877 protein | KIAA0877 | 1.73 | AB020684 |
| 204770_at | transporter 2, ATP- | TAP2 | 1.32 | NM_000544 |
| | binding cassette, sub- | | | |
| | family B (MDR/TAP) | | | |
| 215544_s_at | ortholog of mouse | UBCE7IP5 | 1.40 | AL121891 |
| | ubiquitin conjugating | | | |
| | enzyme 7 interacting pr | | | |
| 215115_x_at | neurotrophic tyrosine | NTRK3 | 1.34 | AI613045 |
| | kinase, receptor, type 3 | | | |
| 216344_at | nephronophthisis 4 | NPHP4 | 1.47 | AL117405 |
| 210501_x_at | large tumor suppressor, | | 1.26 | AF119846 |
| | Drosophila) homolog 1 | | | |
| 200034_s_at | ribosomal protein L6 | RPL6 | 1.28 | NM_000970 |
| 217807_s_at | glioma tumor suppressor | GLTSCR2 | 1.32 | NM_015710 |
| | candidate region gene 2 | | | |
| 200010_at | ribosomal protein L11 | RPL11 | 1.22 | NM_000975 |
| 217740_x_at | ribosomal protein L7a | RPL7A | 1.23 | NM_000972 |
| 201217_x_at | ribosomal protein L3 | RPL3 | 1.33 | NM_000967 |
| 208692_at | ribosomal protein S3 | RPS3 | 1.21 | U14990 |
| 220306_at | hypothetical protein | FLJ20202 | 1.77 | NM_017709 |
| | FLJ20202 | | | |
| 204897_at | prostaglandin E receptor | PTGER4 | 2.54 | NM_000958 |
| | 4 (subtype EP4) | | | |
| 219833_s_at | hypothetical protein | FLJ10466 | 2.20 | NM_018100 |
| | FLJ10466 | | | |
| 212886_at | DKFZP434C171 protein | DKFZP434C171 | 1.87 | AL080169 |
| 60528_at | phospholipase A2, group | PLA2G4B | 1.17 | N71116 |
| | IVB (cytosolic) | | | |
| 214873_at | Homo sapiens mRNA; | DKFZp434O0213 | 2.70 | AL137651 |
| | cDNA | | | |
| 202315_s_at | breakpoint cluster region | BCR | 1.48 | NM_004327 |
| 212715_s_at | KIAA0819 protein | KIAA0819 | 2.56 | AB020626 |
| 204836_at | glycine dehydrogenase | GLDC | 1.67 | NM_000170 |
| 206335_at | galactosamine (N- | GALNS | 1.45 | NM_000512 |
| | acetyl)-6-sulfate sulfatase | | | |
| 216908_x_at | RNA polymerase I | RRN3 | 1.21 | AF001549 |
| | transcription factor | | | |
| | RRN3 | | | |

TABLE 13A
Genes discriminating daunorubicin resistant and sensitive ALL
(B- and T-lineage ALL): Genes down-regulated in daunorubicin resistant
ALL

| | | | | NCBI |
|---|---|---|---|---|
| | | Gene | R/S | Accession |
| Probe ID | Gene Name | Symbol | ratio | Number |
| 213061_s_at | hypothetical protein | LOC123803 | 0.61 | AA643304 |
| | LOC123803 | | | |
| 203112_s_at | Wolf-Hirschhorn | WHSC2 | 0.70 | NM_005663 |
| | syndrome | | | |
| | candidate 2 | | | |
| 218438_s_at | endothelial-derived | EG1 | 0.70 | NM_025205 |
| | gene 1 | | | |
| 202777_at | soc-2 suppressor of | SHOC2 | 0.63 | NM_007373 |
| | clear homolog | | | |
| | (C. elegans) | | | |
| 222251_s_at | glucocorticoid | GMEB2 | 0.71 | AL133646 |
| | modulatory element | | | |
| | binding protein 2 | | | |
| 205070_at | inhibitor of growth | ING3 | 0.76 | NM_019071 |
| | family, member 3 | | | |
| 213264_at | mitogen-activated | MAP3K12 | 0.39 | AW025150 |
| | protein kinase | | | |
| | kinase kinase 12 | | | |
| 218010_x_at | chromosome 20 | C20orf149 | 0.76 | NM_024299 |
| | open reading | | | |
| | frame 149 | | | |

TABLE 13B
Genes discriminating daunorubicin resistant and sensitive ALL
(B- and T-lineage ALL): Genes up-regulated in daunorubicin
resistant ALL

| | | | | NCBI |
|---|---|---|---|---|
| | | Gene | R/S | Accession |
| Probe ID | Gene Name | Symbol | ratio | Number |
| 203517_at | metaxin 2 | MTX2 | 1.60 | NM_006554 |
| 209429_x_at | eukaryotic | EIF2B4 | 1.43 | AF112207 |
| | translation initiation | | | |
| | factor 2B, subunit | | | |
| | 4 delta | | | |
| 204401_at K | intermediate/small | KCNN4 | 1.70 | NM_002250 |
| | conductance | | | |
| | calcium-activated | | | |
| | channel | | | |
| 210423_s_at | solute carrier | SLC11A1 | 1.33 | L32185 |
| | family 11 | | | |
| 204794_at | dual specificity | DUSP2 | 2.09 | NM_004418 |
| | phosphatase 2 | | | |
| 201184_s_at | chromodomain | CHD4 | 1.29 | NM_001273 |
| | helicase DNA | | | |
| | binding protein 4 | | | |
| 207194_s_at | intercellular | ICAM4 | 1.49 | NM_001544 |
| | adhesion molecule 4 | | | |

### C. Expression of Cellular Resistance Genes and Treatment Outcome.

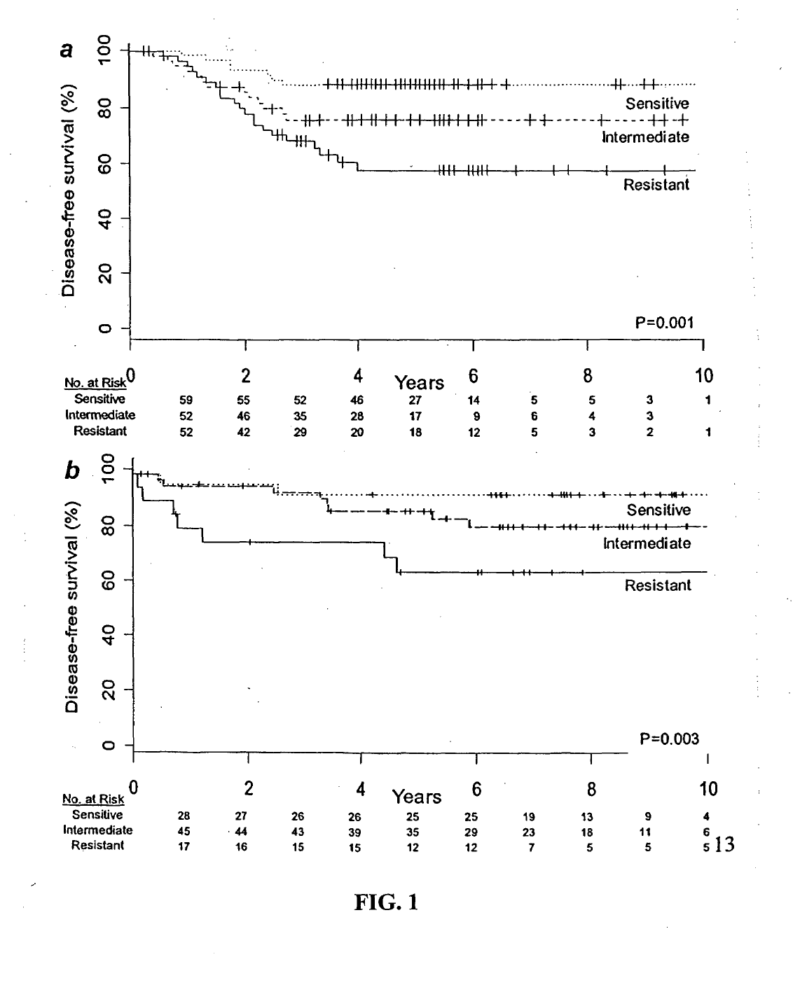

For the 173 patients evaluated, the median follow-up was 4.2 years; 132 remain in continuous complete remission and 40 patients have relapsed and 1 patients had a competing event (second malignancy) that was censored at the time of occurrence. A higher combined gene expression score indicative of resistance to the four drugs, was significantly associated with an increased risk of relapse (FIG. 1a, P=0.001). The combined drug resistance gene expression score was also predictive in a multivariate analysis including known prognostic factors, age and white blood cell count (Hazard ratio=3.39, P=0.007, for patients with a high drug resistance gene expression score versus a low drug resistance gene score.

To assess the robustness of the drug-resistance gene expression profiles in discriminating treatment outcome, the combined gene expression score was tested in a completely independent cohort of 98 patients with acute lymphoblastic leukemia who had been treated with these antileukemic agents, but on a different protocol at St. Jude Children's Research Hospital. The median follow-up of these patients was 7.0 years; 17 had relapsed; 9 had competing events and 72 remained in continuous complete remission. As was the case for patients treated in Europe, a higher combined drug resistance gene expression score was significantly associated with a higher risk of relapse in the independent test set (FIG. 1b, P=0.003). Moreover, when genetic subtypes, lineage, age and white blood cell count at diagnosis were included in a multivariate analysis, the combined drug resistance gene expression score was independently related to a higher probability of disease relapse in this independent test set (Hazard ratio=11.85, P=0.019 for patients with a high drug resistance gene expression score versus a low drug resistance gene score).

### D. Gene Ontology Classification of Discriminating Genes.

Genes discriminating resistance to each antileukemic agent in B-lineage acute lymphoblastic leukemia were grouped in defined functional categories according to the Gene Ontology database. For prednisolone, the percentage of genes involved in metabolism (i.e., carbohydrate metabolism) was higher in the subgroup of 42 discriminating gene probe sets (25 percent) compared to the entire genome (11 percent, P=0.039). For vincristine, genes involved in nucleic acid metabolism (39 percent versus 23 percent, P=0.021) and for L-asparaginase, protein metabolism genes (53 percent versus 20 percent, P<0.001) were over-represented in the group of genes that were associated with drug resistance, compared to the entire genome. Supplemental FIG. 12 depicts the functional groups associated with drug resistance when both immunophenotypes were included in the analyses.

E. Expression of Genes Previously Linked with Drug Resistance or Prognosis in Acute Leukemia.

The great majority of differentially expressed genes that we identified (120 of 123) have not been previously linked to drug resistance for the four agents investigated. Only three genes that were significant in our analyses, (i.e., RPL6, ARHA and SLC2A14) have been previously associated with resistance to doxorubicin (RPL6,25 ARHA,26) or vincristine (SLC2A1427). When the expression of 46 additional genes that encode proteins previously associated with drug resistance or prognosis was compared in sensitive and resistant acute lymphoblastic leukemia, 12 of those genes were differentially expressed for at least one drug at the P<0.05 level, but none reached the level of significance required for inclusion in the models described above. For example, the gene encoding asparagine synthetase (ASNS) was significantly over-expressed in acute lymphoblastic leukemia that was resistant to L-asparaginase, consistent with previously reported differences in the NCI panel of 60 human cancer cell lines (Scherf et al. (2000) Nat. Genet. 24:236-44; and Weinstein et al. (1997) Science 275:343-49). However, ASNS (P=0.0002, Wilcoxon rank sum test; P=0.0005, t-test) was not among the 54 most discriminating probe sets for L-asparaginase sensitivity, as defined by P<0.0001.

### III. Conclusions

The present invention identifies genes that are differentially expressed in acute lymphoblastic leukemia cells that exhibit de novo resistance to widely used antileukemic drugs, and demonstrates that the expression pattern of these genes is related to treatment outcome. The expression of 42, 59, 54 and 22 gene probe sets (representing 123 unique known genes and 30 cDNA clones) in primary B-lineage leukemia cells discriminated cellular resistance to prednisolone, vincristine, L-asparaginase or daunorubicin, respectively. Notably, 120 of the 123 genes discriminating sensitive and resistant acute lymphoblastic leukemia have not been previously associated with cellular resistance to these antileukemic agents to the inventors' knowledge. Twelve genes that have been previously associated with drug resistance or prognosis in acute lymphoblastic leukemia were differentially expressed in acute lymphoblastic leukemia cells resistant to one or more of these drugs (P<0.05), but only three (RPL6, ARHA, SLC2A14) were significant at the level required for inclusion in our models.

No universal “cross-resistance gene” was identified, as no gene was common among genes that discriminated resistance to all four drugs. Discriminating genes belong to numerous functional groups, according to the Gene Ontology (GO) database, and specific functional categories were significantly over-represented for some antileukemic agents. These findings document that resistance to mechanistically distinct antileukemic agents is associated with aberrant expression of different functional groups of genes, in support of combination chemotherapy as the paradigm for cancer treatment. Moreover, these findings point to previously unrecognized targets for developing new agents to augment the efficacy of current chemotherapy acute lymphoblastic leukemia.

Over-expression of the glucose transporter SLC2A14 and glyceraldehyde-3-phosphate dehydrogenase (GAPDH) genes indicates that prednisolone resistant cells have a higher glycolytic rate than prednisolone sensitive cells. This is consistent with the overrepresentation of carbohydrate metabolism-associated genes among those discriminating prednisolone resistance, compared to the human genome. In addition, prednisolone resistance was associated with down-regulation of several transcription-associated genes (PRPF18, SMARCB1 and CTCF). SMARCB1 is a component of the SWI/SNF chromatin remodeling complex, which has been shown to alter nucleosome conformation in an ATP-dependent manner, leading to increased accessibility of nucleosomal DNA to transcription factors (Muchardt and Yaniv (1999) Semin. Cel.l Dev. Biol. 10:189-95. The glucocorticoid receptor is able to recruit the SWI-SNF complex to target promoters, thereby facilitating glucocorticoid-dependent gene activation (Wallberg et al. (2000) Mol. Cell. Biol. 20:2004-13). The current findings indicate that hampering glucocorticoid-dependent gene activation is associated with prednisolone resistance in acute lymphoblastic leukemia.

Vincristine resistance was associated with altered expression of cytoskeleton or extracellular matrix-associated proteins (e.g., TMSB10 and DSC3). Vincristine is cytotoxic by inhibiting tubulin polymerization and disrupting overall cytoskeletal integrity. Over-expression of TMSB10 induces actin depolymerization, resulting in loss of cytoskeletal integrity and apoptosis (Lee et al. (2001) Oncogene 20:6700-6; and Yu et al. (1993) J. Biol. Chem. 268:502-9). It follows that a high basal level of actin depolymerization sensitizes cells to the effects of a tubulin-depolymerizing agent like vincristine. Indeed, vincristine has been found to work synergistically with the actin depolymerizing agent cytochalasin (Kolber and Hill (1992) Cancer Chemother. Pharmacol. 30:286-90). Thus, these findings indicate that modulation of proteins other than tubulin, such as TMSB10, may offer a strategy to sensitize leukemia cells to vinca alkaloids.

L-asparaginase resistance was associated with over-expression of a large group of ribosomal genes (e.g., RPS3, RPL7A and RPL4) and translation-associated genes (e.g., EEFG1, EEF1B2 and EIF3S7). Expression of some ribosomal proteins has been previously linked to doxorubicin resistance in cell lines (Bertram et al. (1998) Eur. J Cancer 34 (5):731-36; and Lopez et al. (2002) Cancer Lett. 180:195-202), but their contribution to L-asparaginase resistance has not been previously recognized to the best of the inventors knowledge. It should be noted that these prior studies determined the expression of only one or two ribosomal protein members, whereas simultaneous over-expression of a large cluster of ribosomal proteins has not been previously linked to cellular drug resistance. L-asparaginase catalyzes the degradation of asparagine, leading to rapid depletion of the circulating pool of asparagine, and consequent diminution of protein synthesis, at least in part by selective suppression of translation of ribosomal proteins (Iiboshi et al. (1999) Biochem. Biophys. Res. Commun. 260:534-39). The current findings suggest that over-expression of ribosomal- and translation-associated genes in acute lymphoblastic leukemia cells confers L-asparaginase resistance. Although it is not intended that the present invention be limited by any particular mechanism, such resistance may be due to overriding the L-asparaginase-induced block of protein synthesis, by over-expressing proteins involved in the translational machinery. Interestingly, under-expression of a different cluster of ribosomal proteins (e.g., RPS11, RPL12 and RPLP2) was associated with vincristine resistance. Taken together, these findings suggest that different ribosomal proteins may contribute to L-asparaginase and vincristine resistance, revealing a potential new mechanism of resistance and suggesting strategies for modulating sensitivity to these antileukemic agents.

We found that down-regulation of the ARHA (RhoA) gene was associated with daunorubicin resistance. Rho proteins, members of the Ras superfamily of GTPases, are important in signal transduction pathways governing cell proliferation and cell death (Van Aelst et al. (1997) Genes Dev. 11:2295-2322). Treatment of leukemic cell lines with daunorubicin induces ceramide generation (Jaffrezou et al. (1996) EMBO J 15:2417-24) and activation of the CD95/CD95-ligand system (Fulda et al. (2000) Blood 95:301-8; and Belaud-Rotureau et al. (2000) Leukemia 14:1266-75. Activation of the latter has been reported to be completely blocked in doxorubicin-resistant leukemic cells. Friesen et al. (1997) Leukemia 11: 1833-41. Over-expression of Rac1, another Rho family member, induces ceramide production and synthesis of CD95-ligand. Embade et al. (2000) Mol. Biol. Cell. 11:4347-58. The present data are the first to link decreased expression of RhoA with daunorubicin resistance, suggesting that RhoA down-regulation impedes daunorubicin-induced proapoptotic signal transduction pathways. A gene that was over-expressed in daunorubicin resistant acute lymphoblastic leukemia was chromodomain helicase DNA-binding protein 4 (CHD4), a central component of the nucleosome remodeling and histone deacetylation (NRD) complex, which leads to transcriptional repression. Tong (1998) Nature 395:917-21. Indeed, the histone deacetylase inhibitor AN-9 has been shown to sensitize non-leukemic cell lines to the cytotoxicity of daunorubicin and doxorubicin (Niitsu et al. (2000) Mol. Pharmacol. 58:27-36), suggesting that targeting CHD4 and/or inhibiting histone deacetylase may be a new strategy to circumvent daunorubicin resistance in pediatric acute lymphoblastic leukemia.

It is noteworthy that the gene expression signatures identified based on the in vitro sensitivity or resistance of primary leukemia cells to the individual antileukemic agents, were related to overall treatment response. Moreover, the robustness of these gene expression signatures was validated by their ability to discriminate outcome in a completely independent population of patients who were treated with these same drugs, but in a separate country on a different protocol. In a multivariate analysis with other known prognostic variables (i.e., age, white blood cell count, genetic subtype and lineage), the combined gene expression score remained significantly related to the risk of disease relapse (Table 14). The four significant variables with P<0.05 were, presence of the BCR/ABL gene fusion (hazard ratio=14.2), combined drug resistance gene expression score (hazard ratio=11.9), age>10 years (hazard ratio=7.6) and white blood cell count>50×109 per L (hazard ratio=10.2). This indicates that the expression of genes identified as conferring drug resistance, is an independent prognostic feature influencing treatment outcome in childhood acute lymphoblastic leukemia.

TABLE 14(a)
Multivariate proportional-hazard analysis of the risk of
disease relapse

| | Number of | Hazard Ratio | |
|---|---|---|---|
| Variable | patients | (95% C.I.) | P-value |
| Age | | | |
| 1-10 | 126 | 1.0* | |
| >10 | 47 | 1.83 (0.96, 3.50) | 0.068 |
| WBC (10⁹/L) | | | |
| <10 | 38 | 1.0* | |
| 10-49 | 68 | 0.98 (0.37, 2.63) | 0.97 |
| 50-99 | 28 | 1.36 (0.42, 4.38) | 0.61 |
| ≧100 | 39 | 4.06 (1.68, 9.77) | 0.002 |
| Combined drug resistance gene | | | |
| expression score‡ | | | |
| Low (<4.70) | 60 | 1.0* | |
| Intermediate (4.70-5.58) | 56 | 3.02 (1.19, 7.70) | 0.020 |
| High (>5.58) | 57 | 3.39 (1.41, 8.17) | 0.007 |

TABLE 14(b)
Multivariate proportional-hazard analysis of the risk of
disease relapse

| | Number of | Hazard Ratio (95% | P- |
|---|---|---|---|
| Variable | patients | C.I.) | value |
| Age | | | |
| 1-10 | 62 | 1.0* | |
| <1 | 7 | 6.48 (0.60, 69.79) | 0.12 |
| >10 | 29 | 7.61 (1.23, 46.95) | 0.029 |
| Genetic Subtype | | | |
| B-lineage other | 24 | 1.0* | |
| BCR-ABL | 8 | 14.17 (2.59, 77.55) | 0.002 |
| E2A-PBX1 | 12 | 0.63 (0.07, 6.29) | 0.69 |
| MLL-AF4 | 14 | 1.23 (0.22, 6.76) | 0.81 |
| TEL-AML1 | 16 | 1.40 (0.09, 21.36) | 0.81 |
| Hyperdiploidy | 15 | 0.92 (0.10, 8.02) | 0.94 |
| T-cell | 9 | 4.03 (0.60, 26.89) | 0.15 |
| WBC (10⁹/L) | | | |
| <10 | 25 | 1.0* | |
| 10-49 | 27 | 1.93 (0.24, 15.15) | 0.53 |
| 50-99 | 20 | 12.02 (1.30, | 0.028 |
| | | 110.79) | |
| ≧100 | 26 | 8.35 (0.84, 83.11) | 0.070 |
| Combined drug resistance gene | | | |
| expression score‡ | | | |
| Low (<4.70) | 29 | 1.0* | |
| Intermediate (4.70-5.58) | 48 | 4.00 (0.58, 27.73) | 0.16 |
| High (>5.58) | 21 | 11.85 (1.51, 93.12) | 0.019 |

The identification of gene expression patterns that confer resistance to individual drugs, reveals proteins and pathways that can be targets for the development of new agents to augment the efficacy of current therapy. Because genes that confer sensitivity or resistance differ for each antileukemic agent, these findings point to strategies whereby one could modulate only the components of therapy to which an individual patient is resistant.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the invention.

That which is claimed:

1. A method of diagnosing drug resistant leukemia in a subject affected by leukemia, the method comprising:
a) providing a subject expression profile of a sample from a subject affected by leukemia;
b) providing a reference expression profile associated with resistance to at least one antileukemic agent selected from prednisolone, vincristine, L-asparaginase, and daunorubicin, wherein the subject expression profile and the reference expression profile comprise one or more values representing the expression level of a gene having differential expression in subjects affected by leukemia who are resistant to the antileukemic agent; and
c) determining whether the subject expression profile shares sufficient similarity to the reference expression profile associated with resistance to the antileukemic agent;
wherein the subject is diagnosed with drug resistant leukemia if the subject expression profile shares sufficient similarity to the reference expression profile associated with resistance to the antileukemic agent.

2. The method of claim 1, wherein the subject expression profile and the reference expression profile comprise values representing the expression levels of at least 20 genes selected from:
(a) the genes shown in Tables 6A, 6B, 6C, 6D, 10A, and 11B;
(b) the genes shown in Tables 7A, 7B, 7C, 7D, 11A, and 11B;
(c) the genes shown in Tables 8A, 8B, 8C, 8D, 12A, and 12B; and
(d) the genes shown in Tables 9A, 9B, 9C, 9D, 13A, and 13B.

3. The method of claim 2 wherein the antileukemic agent is prednisolone and the subject expression profile and the reference expression profile associated with resistance to the antileukemic agent comprise values representing the expression levels of at least 20 genes selected from the genes shown in Tables 6A, 6B, 6C, 6D, 10A, and 10B.

4. The method of claim 2 wherein the antileukemic agent is vincristine and the subject expression profile and the reference expression profile associated with resistance to the drug antileukemic agent comprise values representing the expression levels of at least 20 genes selected from the genes shown in Tables 7A, 7B, 7C, 7D, 11A, and 11B.

5. The method of claim 2 wherein the antileukemic agent is L-asparaginase and the subject expression profile and the reference expression profile associated with resistance to the antileukemic agent comprise values representing the expression levels of at least 20 genes selected from the genes shown in Tables 8A, 8B, 8C, 8D, 12A, and 12B.

6. The method of claim 2 wherein the antileukemic agent is daunorubicin and the subject expression profile and the reference expression profile associated with resistance to the antileukemic agent comprise values representing the expression levels of at least 20 genes selected from the genes shown in Tables 9A, 9B, 9C, 9D, 13A and 13B.

7. The method of claim 1, wherein said sample from said subject affected by leukemia comprises leukemic blasts.

8. The method of claim 1, wherein said sample from said subject affected by leukemia comprises at least 60% leukemic blasts.

9. The method of claim 8, wherein said sample from said subject affected by leukemia comprises at least 75% leukemic blasts.

10. The method of claim 1 wherein said sample comprises leukemic blasts derived from peripheral blood.

11. The method of claim 1 wherein said sample comprises blast cells derived from bone marrow.

12. A method of selecting a therapy for a subject affected by leukemia, said method comprising the steps of:
a) providing a subject expression profile of a sample from said subject affected by leukemia;
b) providing a reference expression profile associated with resistance to at least one antileukemic agent selected from prednisolone, vincristine, L-asparaginase, and daunorubicin, wherein the subject expression profile and the reference expression profile comprise one or more values representing the expression level of a gene having differential expression in subjects affected by leukemia who are resistant to the antileukemic agent; and
c) determining whether the subject expression profile shares sufficient similarity to the reference expression profile associated with resistance to the antileukemic agent;
wherein the therapy selected for the subject does not comprise the antileukemic agent if the subject expression profile shares sufficient similarity to the reference expression profile associated with resistance to the antileukemic agent.

13. The method of claim 12, wherein the subject expression profile and the reference expression profile comprise values representing the expression levels of at least 20 genes selected from:
(a) the genes shown in Tables 6A, 6B, 6C, 6D, 10A, and 11B;
(b) the genes shown in Tables 7A, 7B, 7C, 7D, 11A, and 11B;
(c) the genes shown in Tables 8A, 8B, 8C, 8D, 12A, and 12B; and
(d) the genes shown in Tables 9A, 9B, 9C, 9D, 13A, and 13B.

14. A method of selecting a therapy for a subject affected by leukemia, said method comprising the steps of:
a) providing a subject expression profile of a sample from said subject affected by leukemia;
b) providing a reference expression profile associated with resistance to at least one antileukemic agent selected from prednisolone, vincristine, L-asparaginase, and daunorubicin, wherein the subject expression profile and the reference expression profile comprise one or more values representing the expression level of a gene having differential expression in subjects affected by leukemia who are resistant to the antileukemic agent; and
c) determining whether the subject expression profile is distinguishable from the reference expression profile associated with resistance to the antileukemic agent;
wherein the antileukemic agent is selected as a therapy for the subject if the subject expression profile is distinguishable from the reference expression profile associated with resistance to the antileukemic agent.

15. The method of claim 14, wherein the subject expression profile and the reference expression profile comprise values representing the expression levels of at least 20 genes selected from:
(a) the genes shown in Tables 6A, 6B, 6C, 6D, 10A, and 11B;
(b) the genes shown in Tables 7A, 7B, 7C, 7D, 11A, and 11B;
(c) the genes shown in Tables 8A, 8B, 8C, 8D, 12A, and 12B; and
(d) the genes shown in Tables 9A, 9B, 9C, 9D, 13A, and 13B.

16. A method for screening a library of compounds to identify a compound to improve treatment of drug resistant leukemia, said method comprising the steps of:
a) providing a reference expression profile associated with resistance to at least one antileukemic agent selected from prednisolone, vincristine, L-asparaginase, and daunorubicin, wherein the reference expression profile comprises one or more values representing the expression level of a gene selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B; and
b) providing a cell that is resistant to an antileukemic agent;
c) contacting the cell with one or more compounds from the library of compounds;
d) creating a test expression profile by determining a value representing the expression level in the cell of one or more of the genes whose expression level is represented in the reference expression profile of step a);
(c) determining whether the test expression profile is distinguishable from the reference expression profile;
wherein the compound is identified as a compound useful for improving treatment of drug resistant leukemia if the test expression profile is distinguishable from the reference expression profile.

17. The method of claim 16 wherein the cell that is resistant to antileukemic agents is derived from peripheral blood.

18. The method of claim 16 wherein the cell that is resistant to antileukemic agents is a blast cell derived from bone marrow.

19. The method of claim 16 wherein the antileukemic agent is prednisolone and the subject expression profile and the reference expression profile associated with resistance to the antileukemic agent comprise values representing the expression levels of at least 20 genes selected from the genes shown in Tables 6A, 6B, 6C, 6D, 10A, and 10B.

20. The method of claim 16 wherein the antileukemic agent is vincristine and the subject expression profile and the reference expression profile associated with resistance to the drug antileukemic agent comprise values representing the expression levels of at least 20 genes selected from the genes shown in Tables 7A, 7B, 7C, 7D, 11A, and 11B.

21. The method of claim 16 wherein the antileukemic agent is L-asparaginase and the subject expression profile and the reference expression profile associated with resistance to the antileukemic agent comprise values representing the expression levels of at least 20 genes selected from the genes shown in Tables 8A, 8B, 8C, 8D, 12A, and 12B.

22. The method of claim 16 wherein the antileukemic agent is daunorubicin and the subject expression profile and the reference expression profile associated with resistance to the antileukemic agent comprise values representing the expression levels of at least 20 genes selected from the genes shown in Tables 9A, 9B, 9C, 9D, 13A, and 13B.

23. A method for screening a library of compounds to identify a compound to improve treatment of drug resistant leukemia, said method comprising the steps of:
a) providing a reference expression profile associated with sensitivity to at least one antileukemic agent selected from prednisolone, vincristine, L-asparaginase, and daunorubicin, wherein the reference expression profile comprises one or more values representing the expression level of a gene selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B; and
b) providing a cell that is resistant to an antileukemic agent;
c) contacting the cell with one or more compounds from the library of compounds;
d) creating a test expression profile by determining a value representing the expression level in the cell of one or more of the genes whose expression level is represented in the reference expression profile of step a);
(c) determining whether the test expression profile shares significant similarity with the reference expression profile;
wherein the compound is identified as a compound useful for improving treatment of drug resistant leukemia if the test expression profile shares statistically significant similarity with the reference expression profile.

24. A method for identifying a compound useful for improving treatment of drug resistant leukemia, the method comprising screening a library of compounds to identify a compound that enhances the expression or activity of at least one gene selected from the genes shown in Tables 6A, 6C, 7A, 7C, 8A, 8C, 9A, 9C, 10A, 11A, 12A, and 13A.

25. An improved method for treating drug resistant leukemia comprising the steps of
a) identifying a compound that enhances the expression or activity of at least one gene selected from the genes shown in Tables 6A, 6C, 7A, 7C, 8A, 8C, 9A, 9C, 10A, 11A, 12A, and 13A according to the method of embodiment 24;
b) administering to a subject affected by drug resistant leukemia a therapy comprising an antileukemic agent and a compound identified according to step a).

26. The method of claim 25 wherein the antileukemic agent is prednisolone and the agent that enhances the expression or activity of at least one gene selected from the genes shown in Tables 6A, 6C, and 10A.

27. The method of claim 25 wherein the antileukemic agent is vincristine and the agent that enhances the expression or activity of at least one gene selected from the genes shown in Tables 7A, 7C, and 11A.

28. The method of claim 25 wherein the antileukemic agent is L-asparaginase and the agent that enhances the expression or activity of at least one gene selected from the genes shown in Tables 8A, 8C, and 12A.

29. The method of claim 25 wherein the antileukemic agent is daunorubicin and the agent that enhances the expression or activity of at least one gene selected from the genes shown in Tables 9A, 9C, and 13A.

30. A method for identifying a compound useful for improving treatment of drug resistant leukemia, the method comprising screening a library of compounds to identify a compound that that inhibits the expression or activity of at least one gene selected from the genes shown in Tables 6B, 6D, 7B, 7D, 8B, 8D, 9B, 9D, 10B, 11B, 12B, and 13B.

31. An improved method for treating drug resistant leukemia comprising the steps of
a) identifying a compound that that inhibits the expression or activity of at least one gene selected from the genes shown in Tables 6B, 6D, 7B, 7D, 8B, 8D, 9B, 9D, 10B, 11B, 12B, and 13B according to the method of embodiment 30;
b) administering to a subject affected by drug resistant leukemia a therapy comprising an antileukemic agent and a compound identified according to step a).

32. The method of claim 31 wherein the antileukemic agent is prednisolone and the agent that inhibits the expression or activity of at least one gene selected from the genes shown in Tables 6B, 6D, and 10B.

33. The method of claim 31 wherein the antileukemic agent is vincristine and the agent that inhibits the expression or activity of at least one gene selected from the genes shown in Tables 7B, 7D, and 11B.

34. The method of claim 31 wherein the antileukemic agent is L-asparaginase and the agent that inhibits the expression or activity of at least one gene selected from the genes shown in Tables 8B, 8D, and 12B.

35. The method of claim 31 wherein the antileukemic agent is daunorubicin and the agent that inhibits the expression or activity of at least one gene selected from the genes shown in Tables 9B, 9D, and 13B.

36. An array for use in a method of diagnosing drug resistant leukemia comprising a substrate having a plurality of addresses, wherein each address has disposed thereon a capture probe that can specifically bind a nucleic acid molecule selected from the group consisting of genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B, and wherein the array has fewer than 500 addresses.

37. The array of claim 36, wherein the substrate has greater than 20 addresses.

38. The array of claim 37, wherein the substrate has greater than 40 addresses.

39. The array of claim 38, wherein the substrate has greater than 90 addresses.

40. The array of claim 37 wherein the substrate has fewer than 160 addresses.

41. A kit for diagnosing drug-resistant leukemia comprising:
a) an array according to claim 37; and
b) a computer-readable medium having a plurality of digitally-encoded expression profiles wherein each profile of the plurality has a plurality of values, each value representing the expression of a nucleic acid molecule detected by the array.

42. A computer-readable medium comprising a plurality of digitally-encoded expression profiles wherein each profile of the plurality has at least 20 values, each value representing the expression of a gene selected from the genes shown in Tables 6A, 6B, 6C, 6D, 7A, 7B, 7C, 7D, 8A, 8B, 8C, 8D, 9A, 9B, 9C, 9D, 10A, 10B, 11A, 11B, 12A, 12B, 13A, and 13B.
